(19) United States
(12) Patent Application Publication
(10) Pub. No.: US 2020/0347153 A1
(43) Pub. Date: Nov. 5, 2020

(54) METHODS FOR FORMING INCLUSION COMPLEXES WITH HYDROPHILIC BETA-CYCLODEXTRIN DERIVATIVES AND COMPOSITIONS THEREOF

(71) Applicant: TAKA USA, INC., Carrollton, TX (US)

(72) Inventors: Takako Mohri McGowan, Grapevine, TX (US); Sarah Catherine Hill, Irving, TX (US)

(73) Assignee: TAKA USA, INC., Carrollton, TX (US)

(21) Appl. No.: 16/862,349

(22) Filed: Apr. 29, 2020

Related U.S. Application Data
(60) Provisional application No. 62/881,130, filed on Jul. 31, 2019.
(60) Provisional application No. 62/841,017, filed on Apr. 30, 2019.

(51) Int. Cl. C08B 37/16; A61K 47/69; A61K 8/02; A61K 9/00; A61K 9/06; A61Q 19/00; A61K 8/9789; A61K 31/047; A61K 8/67; A61Q 19/02; A61K 8/35

(52) U.S. Cl. CPC C08B 37/0015; A61K 47/6951; A61K 8/0204; A61K 9/0014; A61K 9/06; A61K 8/35; A61K 8/9789; A61K 31/047; A61K 8/675; A61Q 19/02; A61Q 19/00

(57) ABSTRACT

The described invention provides inclusion complexes of an active agent with a β-cyclodextrin, improved methods for their preparation, methods for characterization of the complexes, and formulation of the complexes as cosmetic compositions or pharmaceutical compositions.

### CROSS-REFERENCE TO RELATED APPLICATIONS

This application claims the benefit of priority to U.S. provisional application 62/881,130 (filed Jul. 31, 2019) and to U.S. provisional application 62/841,017 (filed Apr. 30, 2019). The content of each application is incorporated by reference in its entirety.

### FIELD OF INVENTION

The described invention relates to cyclodextrin inclusion complexes as carriers for lipophilic substances.

### BACKGROUND

Cyclodextrins (CDs) are a group of chemically and physically stable macromolecules produced by enzymatic degradation of starch. They are water-soluble and biocompatible in nature, with a hydrophilic outer surface and lipophilic cavity. They have the shape of a truncated cone or torus (ring shape) rather than a perfect cylinder because of the chair conformation of the glucopyranose units, which are linked by α-(1,4) bonds (Gidwani B, Vyas A. Biomed Res Int. 2015; 198268, citing Merisko-Liversidge E, et al. Eur J Pharm Sci. 2003 February; 18(2): 113-20). CDs consist of six or more glucopyranose units, and are also known as cycloamyloses, cyclomaltoses, and Schardinger dextrins, after an early researcher (Del Valle E M M. Process Biochem. 2004; 39(9): 1033-1046, citing Villiers A. Compt Rendu 1891; 112: 536; Eastburn S D, Tao B Y. Biotechnol Adv 1994; 12: 325-39).

CDs are classified as natural and derived cyclodextrins. Natural cyclodextrins comprise three well-known, industrially produced (major and minor) cyclic oligosaccharides. The most common natural CDs are α, β, and γ, consisting of 6, 7, and 8 glucopyranose units, respectively (Id., citing Nash R A. Cyclodextrins. In: Wade A, Weller P J, editors. Handbook of pharmaceutical excipients. London: Pharm. Press & Am. Pharm. Assoc.; 1994. p. 145-8), although there is evidence for the natural existence of δ-, ζ-, ξ- and even η-cyclodextrin (9-12 residues) (Id., citing Hirose T, Yamamoto Y. Japanese Patent JP 55480 (2001)).

The main interest in cyclodextrins lies in their ability to form inclusion complexes with several compounds (Id., citing Hedges R A. Chem Rev 1998; 98: 2035-44; Lu X, Chen Y. J Chromatogr A 2002; 955: 133-40; Baudin C, et al. Int J Environ Anal Chem 2000; 77: 233-42. Kumar R, et al. Bioresour Technol 2001; 28: 209-11; Koukiekolo R, et al. Eur J Biochem 2001; 268: 841-8). From the X-ray structures it appears that in CDs, the secondary hydroxyl groups (C2 and C3) are located on the wider edge of the ring and the primary hydroxyl groups (C6) on the other edge, and that the apolar C3 and C5 hydrogens and ether-like oxygens are at the inside of the torus-like molecules. This results in a molecule with a hydrophilic outside, which can dissolve in water, and an apolar cavity which provides a hydrophobic matrix, described as a ‘micro heterogeneous environment’ (Id., citing Szetjli J. TIBTRCH 1989; 7: 171-4).

As a result of this cavity, CDs are able to form inclusion complexes with a wide variety of hydrophobic guest molecules. One or two guest molecules can be entrapped by one, two or three cyclodextrins (Id.).

### Properties of Cyclodextrins

The CDs of the three major types: α-cyclodextrin, β-cyclodextrin, and γ-cyclodextrin, are referred to as first generation or parent cyclodextrins. β-Cyclodextrin is the most accessible, the lowest-priced, and generally considered the most useful (Id.). γ-Cyclodextrin is much more soluble in aqueous solutions than β-cyclodextrin, and it possesses relatively good complexing abilities (Loftsson T, Brewster M E. Pharma Tech Eur. 1997; 9: 26-35). The main properties of the major cyclodextrins are given in Table 1 (Del Valle E M M. Process Biochem. 2004; 39(9): 1033-1046).

TABLE 1
Properties of cyclodextrins

| Property | α-Cyclodextrin | β-Cyclodextrin | γ-Cyclodextrin |
|---|---|---|---|
| Number of | 6 | 7 | 8 |
| glucopyranose units | | | |
| Molecular | 972 | 1135 | 1297 |
| weight (g/mol) | | | |
| Solubility | 14.5 | 1.85 | 23.2 |
| in water | | | |
| at 25° C. | | | |
| (%, w/v) | | | |
| Outer diameter | 14.6 | 15.4 | 17.5 |
| (Å) | | | |
| Cavity diameter | 4.7-5.3 | 6.0-6.5 | 7.5-8.3 |
| (Å) | | | |
| Height of torus | 7.9 | 7.9 | 7.9 |
| (Å) | | | |
| Cavity volume | 174 | 262 | 427 |
| (Å)³ | | | |

The natural cyclodextrins have limited aqueous solubility and their complex formation with lipophilic drugs often results in precipitation of solid drug-cyclodextrin complexes. For example, the solubility of β-cyclodextrin in water is only approximately 19 mg/mL at room temperature. This low aqueous solubility is, at least partly, associated with strong intramolecular hydrogen bonding in the cyclodextrin crystal lattice. Substitution of any of the hydrogen bond-forming hydroxyl groups, even by hydrophobic moieties such as methoxy groups, will increase the aqueous solubility of β-cyclodextrin (Loftsson T, Brewster M E. Pharma Tech Eur. 1997; 9: 26-35).

Studies of cyclodextrins in solution are supported by a large number of crystal structure studies. Cyclodextrins crystallize in two main types of crystal packing, channel structures and cage structures, depending on the type of cyclodextrin and guest compound (Del Valle E M M. Process Biochem. 2004; 39(9): 1033-1046).

These crystal structures show that cyclodextrins in complexes adopt the expected ‘round’ structure with all glucopyranose units in the ⁴C₁ chair conformation. Furthermore, studies with linear maltohexaoses, which form an antiparallel double helix, indicate that α-cyclodextrin is the form in which the steric strain (meaning the increase in potential energy of a molecule due to repulsion between electrons in atoms that are not directly bonded to each other) due to cyclization is least while γ-cyclodextrin is most strained (Id., citing Szetjli J. Chem Rev 1998; 98: 1743-53).

Apart from these naturally occurring cyclodextrins, many cyclodextrin derivatives have been synthesized. These derivatives usually are produced by aminations, esterifications or etherifications of primary and secondary hydroxyl groups of the cyclodextrins. Depending on the substituent, the solubility of the cyclodextrin derivatives is usually different from that of their parent cyclodextrins. Virtually all derivatives have a changed hydrophobic cavity volume, and these modifications can improve solubility, stability against light or oxygen, and help control the chemical activity of guest molecules (Id., citing Villiers A. Compt Rendu 1891; 112: 536).

In addition, as these manipulations frequently produce large numbers of isomeric products, chemical modification can transform the crystalline cyclodextrins into amorphous mixtures, increasing their aqueous solubility and complexity (Loftsson T, Brewster M E. Pharma Tech Eur. 1997; 9: 26-35, citing Pitha J, et al. Intl J Pharm. (1986) 29: 73-82). For example, isomeric mixtures of 2-hydroxypropyl-β-cyclodextrin are obtained by treating a base-solubilized solution of β-cyclodextrin with propylene oxide. The aqueous solubility of 2-hydroxypropyl-β-cyclodextrin is more than 60 g/100 mL (Id., citing Frömming K-H, Szejtli. Cyclodextrins in Pharmacy; Kluwer Academic Publishers, Dordrecht, The Netherlands, 1994; Pitha J, et al. Intl J Pharm. (1986) 29: 73-82). Both the molar substitution, that is, the average number of propylene oxide molecules that have reacted with one glucopyranose unit, and the location of the hydroxypropyl groups on the β-cyclodextrin molecule will affect the complexing properties of the 2-hydroxypropyl-β-cyclodextrin mixture (Id.).

The pharmaceutical safety of many of the cyclodextrins currently available has been examined (Id., citing Irie T, Uekama K. J Pharm Sci 1997; 86: 147-162; Frömming K-H, Szejtli. Cyclodextrins in Pharmacy; Kluwer Academic Publishers, Dordrecht, The Netherlands, 1994; Duchêne D, Wouessidjewe D. Pharmaceutical and Medical Applications of Cyclodextrins, in S. Dumitriu, Ed., Polysaccharides in Medical Applications; Marcel Dekker, New York, USA, 1996: 575-602). Topical and oral administration of the parent α-, β- and γ-cyclodextrins, as well as that of their hydrophilic derivatives (for example, 2-hydroxypropyl-β-cyclodextrin, sulfobutylether β-cyclodextrin and maltosyl-β-cyclodextrin) is considered to be safe in most circumstances. Hydrophilic cyclodextrins poorly penetrate lipophilic biological membranes, meaning that they have negligible oral, dermal or ocular bioavailability (Id., citing Hirayama F, Uekama K. Methods of Investigating and Preparing Inclusion Compounds, in D. Duchêne, Ed., Cyclodextrins and Their Industrial Uses; Editions de Sante, Paris, France, 1987: 131-172). These materials represent, therefore, true drug carriers. γ-Cyclodextrin, and the hydrophilic β-cyclodextrin derivatives (for example, 2-hydroxypropyl-β-cyclodextrin and probably sulfobutylether β-cyclodextrin) can be used in parenteral dosage forms based on their documented intravenous safety. β-Cyclodextrin and its lipophilic, water-soluble, methylated derivatives cannot be used in parenteral dosage forms. The limited water solubility of β-cyclodextrin causes the compound to precipitate in the kidney, which can induce nephrotoxicity, and the lipophilic cyclodextrins exert detergent-like effects and destabilize biological membranes, including red blood cells (Id.)

Cyclodextrins are frequently used as building blocks. Up to 20 substituents have been linked to β-cyclodextrin in a regioselective manner (meaning the process that favors bond formation at a particular atom over other possible atoms). The synthesis of uniform cyclodextrin derivatives requires regioselective reagents, optimization of reaction conditions and a good separation of products. The most frequently studied reaction is an electrophilic attack at the OH-groups. The formation of ethers and esters by alkyl halides, epoxides, acyl derivatives, isocyanates, and by inorganic acid derivatives as sulphonic acid chloride cleavage of C—OH bonds has also been studied frequently, involving a nucleophilic attack by compounds such as azide ions, halide ions, thiols, thiourea, and amines; this requires activation of the oxygen atom by an electron-withdrawing group (Id., citing Szetjli J. Chem Rev 1998; 98: 1743-53).

Because of their ability to link covalently or noncovalently specifically to other cyclodextrins, cyclodextrins can be used as building blocks for the construction of supramolecular complexes. Their ability to form inclusion complexes with organic host molecules offers possibilities to build supra molecular threads. In this way molecular architectures such as catenanes, rotaxanes, polyrotaxanes, and tubes, can be constructed. Such building blocks, which cannot be prepared by other methods, can be employed, for example, for the separation of complex mixtures of molecules and enantiomers (Del Valle E M M. Process Biochem. 2004; 39(9): 1033-1046, citing Szetjli J. Chem Rev 1998; 98: 1743-53).

### Inclusion Complex Formation

The most notable feature of cyclodextrins is their ability to form solid inclusion complexes (host-guest complexes) with a very wide range of solid, liquid and gaseous compounds by a molecular complexation (Id., citing Villiers A. Compt Rendu 1891; 112: 536). In these complexes, a guest molecule is held within the cavity of the cyclodextrin host molecule. Complex formation is a dimensional fit between host cavity and guest molecule (Id., citing Muñoz-Botella S, et al. Ars Pharm 1995; 36: 187-98). The lipophilic cavity of cyclodextrin molecules provides a microenvironment into which appropriately sized non-polar moieties can enter to form inclusion complexes (Id., citing Loftsson T, Brewster M E. J Pharm Sci 1996; 85: 1017-25). No covalent bonds are broken or formed during formation of the inclusion complex (Id., citing Schneiderman E, Stalcup A M. J Chromatogr B 2000; 745: 83-102). The main driving force of complex formation is the release of enthalpy-rich water molecules from the cavity. Water molecules are displaced by more hydrophobic guest molecules present in the solution to attain an apolar-apolar association and decrease of cyclodextrin ring strain resulting in a more stable lower energy state (Id., citing Szetjli J. Chem Rev 1998; 98: 1743-53).

The binding of guest molecules within the host cyclodextrin is not fixed or permanent but rather is a dynamic equilibrium. Binding strength depends on how well the ‘host-guest’ complex fits together and on specific local interactions between surface atoms. Complexes can be formed either in solution or in the crystalline state, and water is typically the solvent of choice. Inclusion complexation can be accomplished in a co-solvent system and in the presence of any non-aqueous solvent. Cyclodextrin architecture confers upon these molecules a wide range of chemical properties markedly different from those exhibited by non-cyclic carbohydrates in the same molecular weight range (Id.).

Inclusion in cyclodextrins exerts a profound effect on the physicochemical properties of guest molecules as they are temporarily locked or caged within the host cavity giving rise to beneficial modifications of guest molecules, which are not achievable otherwise (Id., citing Schmid G. Trends Biotechnol 1989; 7: 244-8). These properties are: solubility enhancement of highly insoluble guests, stabilization of labile guests against the degradative effects of oxidation, visible or UV light and heat, control of volatility and sublimation, physical isolation of incompatible compounds, chromatographic separations, taste modification by masking off flavors, unpleasant odors and controlled release of drugs and flavors. Therefore, cyclodextrins are used in food (Id., citing Fujishima N, et al. Japanese Patent JP 136,898 (2001)), pharmaceuticals (Id., citing Bhardwaj R, et al. J Pharm Sci Technol 2000; 54: 233-9), cosmetics (Id., citing Holland L, et al. PCT Int Appl WO 67,716 (1999)), environment protection (Id., citing Lezcano M, et al. J Agric Food Chem 2002; 50: 108-12, bioconversion (Id., citing Dufosse L, et al. Biotechnol Prog 1999; 15: 135-9), packing and the textile industry (Id., citing Hedges R A. Chem Rev 1998; 98: 2035-44).

The potential guest list for molecular encapsulation in cyclodextrins is quite varied, and includes such compounds as straight or branched chain aliphatics, aldehydes, ketones, alcohols, organic acids, fatty acids, aromatics, gases, and polar compounds, such as halogens, oxyacids and amines (Id., citing Schmid G. Trends Biotechnol 1989; 7: 244-8). Due to the availability of multiple reactive hydroxyl groups, the functionality of cyclodextrins is greatly increased by chemical modification. Through modification, the applications of cyclodextrins are expanded. CDs are modified through substituting various functional compounds on the primary and/or secondary face of the molecule. For example, modified CDs are useful as enzyme mimics because the substituted functional groups act in molecular recognition. The same property is used for targeted drug delivery and analytical chemistry, as modified CDs show increased enantioselectivity over native CDs (Id., citing Villiers A. Compt Rendu 1891; 112: 536).

The ability of a cyclodextrin to form an inclusion complex with a guest molecule is a function of two key factors. The first is steric, and depends on the relative size of the cyclodextrin compared to the size of the guest molecule or certain key functional groups within the guest. If the guest is the wrong size, it will not fit properly into the cyclodextrin cavity. The second critical factor is the thermodynamic interactions between the different components of the system (cyclodextrin, guest, solvent). For a complex to form, there must be a favorable net energetic driving force that pulls the guest into the cyclodextrin (Id.).

While the height of the cyclodextrin cavity is the same for all three types, the number of glucose units determines the internal diameter of the cavity and its volume. Based on these dimensions, α-cyclodextrin can typically complex low molecular weight molecules or compounds with aliphatic side chains, β-cyclodextrin will complex aromatics and heterocycles, and γ-cyclodextrin can accommodate larger molecules such as macrocycles and steroids (Id.).

In general, there are four energetically favorable interactions that help shift the equilibrium to form the inclusion complex: (1) the displacement of polar water molecules from the apolar cyclodextrin cavity; (2) the increased number of hydrogen bonds formed as the displaced water returns to the larger pool; (3) a reduction of the repulsive interactions between the hydrophobic guest and the aqueous environment; and (4) an increase in the hydrophobic interactions as the guest inserts itself into the apolar cyclodextrin cavity (Id.).

While this initial equilibrium to form the complex is very rapid (often within minutes), the final equilibrium can take much longer to reach. Once inside the cyclodextrin cavity, the guest molecule makes conformational adjustments to take maximum advantage of the weak van der Waals forces that exist (Id.).

Dissociation of the inclusion complex is a relatively rapid process usually driven by a large increase in the number of water molecules in the surrounding environment. The resulting concentration gradient shifts the equilibrium to the left. In highly dilute and dynamic systems like the body, the guest has difficulty finding another cyclodextrin to reform the complex and is left free in solution (Id.).

### Equilibrium

The central cavity of the cyclodextrin molecule is lined with skeletal carbons and ethereal oxygens of the glucose residues. It is, therefore, lipophilic. The polarity of the cavity has been estimated to be similar to that of aqueous ethanolic solution (Id., citing Frömming KH, Szejtli J. Cyclodextrins in pharmacy. Topics in inclusion science. Dordrecht: Kluwer Academic Publishers; 1994). It provides a lipophilic microenvironment into which suitably sized drug molecules may enter and include. Usually, one drug molecule forms a complex with one cyclodextrin molecule.

Measurements of stability or equilibrium constants (K_{c}) or the dissociation constants (K_{d}) of the drug-cyclodextrin complexes are important since this is an index of changes in physicochemical properties of a compound upon inclusion. Most methods for determining the K-values are based on titrating changes in the physicochemical properties of the guest molecule, e.g, a drug molecule, with the cyclodextrin and then analyzing the concentration dependencies. Additive properties that can be titrated in this way to provide information on the K-values include aqueous solubility (Id., citing Hirayama F, Uekama K. Methods of investigating and preparing inclusion compounds. In: Duchêne D, editor. Cyclodextrins and their industrial uses. Paris: Editions de Santé; 1987. p. 131-72; Higuchi T, Connors K A. Adv Anal Chem Instrum 1965; 4: 117-212; Sigurdardottir A M, Loftsson T. Int J Pharm 1995; 126: 73-8; Hussain M A, et al. J Pharm Sci 1993; 82: 77-9), chemical reactivity (Id., citing Loftsson T. Drug Stabil 1995; 1: 22-33; Másson M, et al. Int J Pharm 1998; 164: 45-55), molar absorptivity and other optical properties (e.g. optical rotation dispersion), phase solubility measurements (Id., citing Liu F, et al. Pharm Res 1992; 9: 1671-2), nuclear magnetic resonance chemical shifts, pH-metric methods, calorimetric titration, freezing point depression (Id., citing Suzuki M, et al. Chem Pharm Bull 1993; 41: 1616-20), and liquid chromatography chromatographic retention times. While it is possible to use both guest or host changes to generate equilibrium constants, guest properties are usually most easily assessed.

### Complex Formation

Cyclodextrin inclusion is a stoichiometric molecular phenomenon in which usually only one guest molecule interacts with the cavity of a cyclodextrin molecule to become entrapped. In the case of some low molecular weight molecules, more than one guest molecule may fit into the cavity, and in the case of some high molecular weight molecules, more than one cyclodextrin molecule may bind to the guest. In principle, only a portion of the molecule must fit into the cavity to form a complex. As a result, one-to-one molar ratios are not always achieved, especially with high or low molecular weight guests. A variety of non-covalent forces, such as van der Waals forces, hydrophobic interactions and other forces, are responsible for the formation of a stable complex (Id.).

Complexes can be formed by a variety of techniques that depend on the properties of the active material, the equilibrium kinetics, the other formulation ingredients and processes and the final dosage form desired. However, each of these processes depends on a small amount of water to help drive the thermodynamics. Among the methods used are simple dry mixing, mixing in solutions and suspensions followed by a suitable separation, the preparation of pastes and several thermo-mechanical techniques (Id.).

In the crystalline form, only the surface molecules of the cyclodextrin crystal are available for complexation. In solution, more cyclodextrin molecules become available. Heating increases the solubility of the cyclodextrin as well as that of the guest, and this increases the probability of complex formation. Complexation occurs more rapidly when the guest compound is either in soluble form or in dispersed fine particles (Id.). Complexes can be prepared by the addition of an excess amount of a drug to an aqueous cyclodextrin solution (Loftsson T, Brewster M E. Pharma Tech Eur. 1997; 9: 26-35). The suspension formed is equilibrated (for periods of up to one week at the desired temperature) and then filtered or centrifuged to form a clear drug-cyclodextrin complex solution. Since the rate-determining step in complex formation is often the phase-to-phase transition of the drug molecule, it is sometimes possible to shorten this process by formation of supersaturated solutions through sonication followed by precipitation. For preparation of the solid complexes, the water is removed from the aqueous drug-cyclodextrin solutions by evaporation or sublimation, for example spray-drying or freeze-drying (Id.).

Temperature has more than one effect upon cyclodextrin complexes. Heating can increase the solubility of the complex but, at the same time also destabilizes the complex. These effects often need to be balanced. As heat stability of the complex varies from guest to guest, most complexes start to decompose at 50° C.-60° C., while some complexes are stable at higher temperatures, especially if the guest is strongly bound or the complex is highly insoluble (Del Valle E M M. Process Biochem. 2004; 39(9): 1033-1046).

Water is the most commonly used solvent in which complexation reactions are performed. The more soluble the cyclodextrin in the solvent, the more molecules become available for complexation. The guest must be able to displace the solvent from the cyclodextrin cavity if the solvent forms a complex with the cyclodextrin. Water, for example is very easily displaced. The solvent must be easily removed if solvent-free complexes are desired. In the case of multi-component guests, one of the components may act as a solvent and be included as a guest. Not all guests are readily solubilized in water, making complexation either very slow or impossible. In such cases, an organic solvent can be used to dissolve the guest. The solvent should not complex well with cyclodextrin and should be easily removed by evaporation. Ethanol and diethyl ether are good examples of such solvents (Id.).

As the amount of water is increased, the solubility of both cyclodextrin and guest are increased so that complexation occurs more readily. However, as the amount of water is further increased, the cyclodextrin and the guest may be so dilute that they do not get in contact as easily as they do in a more concentrated solution. Therefore, it is desirable to keep the amount of water sufficiently low to ensure complexation occurs at a sufficiently fast rate (Id.).

Some high molecular weight compounds such as oils have a tendency to associate with themselves rather than interacting with cyclodextrin. In such cases, more water allied with good mixing can allow better dispersion and separation of the oil molecules or isolation of the oil molecules from each other. When the oil molecules come into contact with the cyclodextrin, they form a more stable complex than they would if less water were present (Id.).

Volatile guests can be lost during complexation, especially if heat is used. With highly volatile guests, this can be prevented by using a sealed reactor or by refluxing the volatile guests back to the mixing vessel (Id.).

Other methods, including co-precipitation, neutralization and kneading and grinding techniques, can also be applied to prepare solid drug-cyclodextrin complexes (Loftsson T, Brewster M E. Pharma Tech Eur. 1997; 9: 26-35, citing Hirayama F, Uekama K. Methods of Investigating and Preparing Inclusion Compounds, in D. Duchêne, Ed., Cyclodextrins and Their Industrial Uses; Editions de Sante, Paris, France, 1987: 131-172). In the kneading method, the drug is added to an aqueous slurry of a poorly water-soluble cyclodextrin, such as β-cyclodextrin. The mixture is thoroughly mixed, often at elevated temperatures, to yield a paste which is then dried (Id., citing Hirayama F, Uekama K. Methods of Investigating and Preparing Inclusion Compounds, in D. Duchêne, Ed., Cyclodextrins and Their Industrial Uses; Editions de Sante, Paris, France, 1987: 131-172). This technique can frequently be modified so that it can be accomplished in a single step with the aid of commercially available mixers that can be operated at temperatures of more than 100° C. and under vacuum. The kneading method is a cost-effective means for preparing solid cyclodextrin complexes of poorly water-soluble drugs (Id.).

Co-precipitation is the most widely used method in the laboratory (Del Valle E M M. Process Biochem. 2004; 39(9): 1033-1046). Cyclodextrin is dissolved in water and the guest is added while stirring the cyclodextrin solution. The concentration of β-cyclodextrin can be as high as about 20% if the guest can tolerate higher temperatures. If a sufficiently high concentration is chosen, the solubility of the cyclodextrin-guest complex will be exceeded as the complexation reaction proceeds or as cooling is applied. In many cases, the solution of cyclodextrin and guest must be cooled while stirring before a precipitate is formed. The precipitate can be collected by decanting, centrifugation or filtration. The precipitate may be washed with a small amount of water or other water-miscible solvent such as ethyl alcohol, methanol or acetone (Id.). Organic solvents used as precipitants can interfere with complexation which makes this approach less attractive (Loftsson T, Brewster M E. Pharma Tech Eur. 1997; 9: 26-35).

The main disadvantage of the co-precipitation method lies in the scale-up. Because of the limited solubility of the cyclodextrin, large volumes of water have to be used. Tank capacity, time and energy for heating and cooling may become important cost factors. Treatment and disposal of the mother liquor obtained after collecting the complex may also be a concern. This can be diminished in many cases by recycling the mother liquor (Del Valle E M M. Process Biochem. 2004; 39(9): 1033-1046, citing Loftsson T, et al. Eur J Pharm Sci 1993; 1: 95-101; Pitha J, Hoshino T. Int J Pharm 1992; 80: 243-51). In addition, non-ionic surfactants have been shown to reduce cyclodextrin complexation of diazepam and preservatives to reduce the cyclodextrin complexation of various steroids (Id., citing Loftsson T, et al. Drug Devel Ind Pharm 1992; 18(13): 1477-84). On the other hand, additives such as ethanol can promote complex formation in the solid or semisolid state (Id., citing Furuta T, et al. Supramol Chem 1993; 1: 321-5). Un-ionized drugs usually form a more stable cyclodextrin complex than their ionic counterparts and, thus, complexation efficiency of basic drugs can be enhanced by addition of ammonia to the aqueous complexation media. For example, solubilization of pancratistatin with hydroxypropyl-cyclodextrins was optimized upon addition of ammonium hydroxide (Id., citing Torres-Labandeira J J, et al. J Pharm Sci 1990; 80: 384-6).

In slurry complexation, it is not necessary to dissolve the cyclodextrin completely to form a complex. Cyclodextrin can be added to water as high as 50-60% solids and stirred. The aqueous phase will be saturated with cyclodextrin in solution. Guest molecules will complex with the cyclodextrin in solution and, as the cyclodextrin complex saturates the water phase, the complex will crystallize or precipitate out of the aqueous phase. The cyclodextrin crystals will dissolve and continue to saturate the aqueous phase to form the complex and precipitate or crystallize out of the aqueous phase, and the complex can be collected in the same manner as with the co-precipitation method. The amount of time required to complete the complexation is variable, and depends on the guest. Assays must be done to determine the amount of time required. Generally, slurry complexation is performed at ambient temperatures. With many guests, some heat may be applied to increase the rate of complexation, but care must be applied since too much heat can destabilize the complex and the complexation reaction may not be able to take place completely. The main advantage of this method is the reduction of the amount of water needed and the size of the reactor (Id.).

Paste complexation is a variation of the slurry method. Only a small amount of water is added to form a paste, which is mixed with the cyclodextrin using a mortar and pestle, or on a large scale using a kneader. The amount of time required is dependent on the guest. The resulting complex can be dried directly or washed with a small amount of water and collected by filtration or centrifugation. Pastes will sometimes dry forming a hard mass instead of a fine powder. This is dependent on the guest and the amount of water used in the paste. Generally, the hard mass can be dried thoroughly and milled to obtain a powdered form of the complex (Id.).

Damp mixing and heating uses little or no added water. The amount of water can range from the amount of water of hydration in the cyclodextrin and added guest to up to 20-25% water on a dry basis. This amount of water is typically found in a filter cake from the co-precipitation or slurry methods. The guest and cyclodextrin are thoroughly mixed and placed in a sealed container. The sealed container and its contents are heated to about 100° C. and then the contents are removed and dried. The amount of water added, the degree of mixing and the heating time have to be optimized for each guest (Id.).

Extrusion is a variation of the heating and mixing method and is a continuous system. Cyclodextrin, guest and water can be premixed or mixed as added to the extruder. Degree of mixing, amount of heating and time can be controlled in the barrel of the extruder. Depending upon the amount of water, the extruded complex may dry as it cools or the complex may be placed in an oven to dry. Extrusion has the advantages of being a continuous process and of using very little water. Because of the heat generated, some heat-labile guests decompose using this method (Id.).

Some guests can be complexed by simply adding guest to the cyclodextrin and dry mixing them together. This works best with oils or liquid guests. The amount of mixing time required is variable and depends on the guest. Generally, this method is performed at ambient temperature and is a variation on the paste method. The main advantage is that no water need be added, unless a washing step is used. Its disadvantages are the risk of caking on scale-up, resulting in mixing not being sufficiently thorough leading to incomplete complexation, and, with many guests, the length of time required (Id.).

Solid complexes of ionizable drugs can sometimes be prepared by the neutralization method, wherein the drug is dissolved in an acidic (for basic drugs) or basic (for acidic drugs) aqueous cyclodextrin solution. The solubility of the drug is then lowered through appropriate pH adjustments (that is, formation of the unionized drug) to force the complex out of solution. Solid drug-cyclodextrin complexes can also be formed by the grinding of a physical mixture of the drug and cyclodextrin and then heating the mixture in a sealed container to 60° C.-90° C. (Loftsson T, Brewster M E. Pharma Tech Eur. 1997; 9: 26-35, citing Nakai Y, et al. Chem Pharm Bull 1991; 39: 1532-1535).

Complexes can also be spray-dried. Precipitation must be controlled in order to avoid the particles becoming too large and blocking the atomizer or spray nozzle. With volatile guests, some optimization of drying conditions is required in order to reduce the losses. Spray drying is not a viable means for drying highly volatile and heat-labile guests (Del Valle E M M. Process Biochem. 2004; 39(9): 1033-1046).

### Release

Once a complex is formed and dried, generally it is very stable, exhibiting long shelf life at ambient temperatures under dry conditions. Displacement of the complexed guest by another guest requires heating. In many cases, water can replace the guest. When a complex is placed in water, two steps are involved in the release of the complexed guest. First, the complex is dissolved. The second step is the release of the complexed guest when displaced by water molecules. An equilibrium will be established between free and complexed cyclodextrin, the guest and the dissolved and undissolved complex. In the case of complexes containing multiple guest components or cyclodextrin types, guest molecules are not necessarily released in the same proportion as in the original guest mixture. Each guest complex may have different solubility and rate of release from the complex. If release rates are different for each component, it is possible to obtain an intended release pattern by alteration of the guest formulation (Id.).

### Applications of Cyclodextrins

The characteristics of cyclodextrins and their derivatives make them suitable for applications in analytical chemistry, agriculture, the pharmaceutical field, and in food and toiletry articles (Id., citing Singh M, et al. Biotechnol Adv 2002; 20: 341-59).

### Cosmetics, Personal Care and Toiletry

Cyclodextrin use has proved beneficial in volatility suppression of perfumes, room fresheners and detergents by controlled release of fragrances from inclusion compounds. The major benefits of cyclodextrins in this sector are stabilization, odor control and process improvement upon conversion of a liquid ingredient to a solid form. Applications include toothpaste, skin creams, liquid and solid fabric softeners, paper towels, tissues and underarm shields. The interaction of the guest with CDs produces a higher energy barrier to overcome to volatilize, thus producing long-lasting fragrances (Id., citing Prasad N, et al. European Patent 1,084,625; 1999). Fragrance is enclosed with the CD and the resulting inclusion compound is complexed with calcium phosphate to stabilize the fragrance in manufacturing bathing preparations (Id., citing Tatsuya S. Japanese Patent 11,209,787; 1999). Holland et al. (1999) prepared cosmetic compositions containing CDs to create long-lasting fragrances (Id., citing Holland L, et al. PCT Int Appl WO 67,716; 1999). CD-based compositions are also used in various cosmetic products to reduce body odors (Id., citing Trinh J, et al. U.S. Pat. No. 5,897,855; 1999). The major benefits of CDs in this sector are stabilization, odor control, process improvement upon conversion of a liquid ingredient to a solid form, flavor protection and flavor delivery in lipsticks, water solubility and enhanced thermal stability of oils (Id., citing Buschmann H J, Schollmeyer E. J Cosmet Sci 2002; 53: 575-92). Some of the other applications include use in toothpaste, skin creams, liquid and solid fabric softeners, paper towels, tissues and underarm shields (Id., citing Szetjli J. Chem Rev 1998; 98: 1743-53).

The use of CD-complexed fragrances in skin preparations such as talcum powder stabilizes the fragrance against the loss by evaporation and oxidation over a long period. The antimicrobial efficacy of the product is also improved (Id., citing Hedges R A. Chem Rev 1998; 98: 2035-44). Dry CD powders of size less than 12 mm are used for odor control in diapers, menstrual products, paper towels, etc. and are also used in hair care preparations for the reduction of volatility of odorous mercaptans. The hydroxypropyl β-cyclodextrin surfactant, either alone or in combination with other ingredients, provides improved antimicrobial activity (Id., citing Woo RAM, et al. U.S. Pat. No. 5,942,217; 1999). Dishwashing and laundry detergent compositions with CDs can mask odors in washed items (Id., citing Foley P R, et al. PCT Int Appl WO 01 23,516; 2000; Angell W F, France, P A. PCT Int Appl WO 01 18,163; 2001). CDs used in silica-based toothpastes increase the availability of triclosan (an antimicrobial) by cyclodextrin complexation, resulting in an almost threefold enhancement of triclosan availability (Id., citing Loftsson T, et al. J Pharm Sci 1999; 88: 1254-8). CDs are used in the preparation of sunscreen lotions in 1:1 proportion (sunscreen/hydroxypropyl β-CD) as the CD's cavity limits the interaction between the UV filter and the skin, reducing the side effects of the formulation. Similarly, by incorporating CDs in self-tanning emulsions or creams, the performance and shelf life are improved. An added bonus is that the tan looks more natural than the yellow and reddish tinge produced by traditional dihydroxyacetone products (Id., citing Scalia S, et al. J Pharm Pharmacol 1999; 51: 1367-74).

### Foods and Flavors

Cyclodextrins are used in food formulations for flavor protection or flavor delivery. They form inclusion complexes with a variety of molecules including fats, flavors and colors. Most natural and artificial flavors are volatile oils or liquids and complexation with cyclodextrins provides a promising alternative to the conventional encapsulation technologies used for flavor protection. Cyclodextrins are also used as process aids, for example, to remove cholesterol from products such as milk, butter and eggs. Cyclodextrins were reported to have a texture-improving effect on pastry and on meat products. Other applications arise from their ability to reduce bitterness, ill smell and taste and to stabilize flavors when subjected to long-term storage. Emulsions like mayonnaise, margarine or butter creams can be stabilized with α-cyclodextrin. β-cyclodextrin may be used to remove cholesterol from milk, to produce dairy products low in cholesterol (Id., citing Szetjli J. Chem Rev 1998; 98: 1743-53; Hedges R A. Chem Rev 1998; 98: 2035-44).

Cyclodextrins act as molecular encapsulants, protecting the flavor throughout many rigorous food-processing methods of freezing, thawing and microwaving. β-CD as a molecular encapsulant allows the flavor quality and quantity to be preserved to a greater extent and longer period compared to other encapsulants and provides longevity to the food item (Id., citing Loftsson T, Brewster M E. J Pharm Sci 1996; 85: 1017-25). In Japan, cyclodextrins have been approved as ‘modified starch’ for food applications for more than two decades, serving to mask odors in fresh food and to stabilize fish oils. Some European countries, for example Hungary, have approved γ-cyclodextrin for use in certain applications because of its low toxicity (Id.).

The complexation of CDs with sweetening agents such as aspartame stabilizes and improves the taste. It also eliminates the bitter aftertaste of other sweeteners such as stevioside, glycyrrhizin and rubusoside. Enhancement of flavor by CDs has been also claimed for alcoholic beverages such as whisky and beer (Id., citing Parrish M A. Cyclodextrins-a review. England: Sterling Organics; 1988; Newcastle-upon-Tyne NE3 3TT). The bitterness of citrus fruit juices is a major problem in the industry caused by the presence of limonoids (mainly limonin) and flavonoids (mainly naringin). Cross-linked cyclodextrin polymers are useful to remove these bitter components by inclusion complexes (Id.).

The most prevalent use of CD in process aids is the removal of cholesterol from animal products such as eggs, dairy products. CD-treated material shows 80% removal of cholesterol. Free fatty acids can also be removed from fats using CDs, thus improving the frying property of fat (e.g. reduced smoke formation, less foaming, less browning and deposition of oil residues on surfaces) (Id., citing Hedges R A. Chem Rev 1998; 98: 2035-44). Fruits and vegetable juices are also treated with CD to remove phenolic compounds, which cause enzymatic browning. In juices, polyphenol-oxidase converts the colorless polyphenols to color compounds, and addition of CDs removes polyphenoloxidase from juices by complexation. Sojo et al. (1999) studied the effect of cyclodextrins on the oxidation of o-diphenol by banana polyphenol oxidase and found that cyclodextrins act as activator as well as inhibitor (Id., citing Sojo M M, et al. J Agric Food Chem 1999; 47: 518-23). By combining 1-4% CD with chopped ginger root, Sung (1997) established that it can be stored in a vacuum at cold temperature for 4 weeks or longer without browning or rotting (Id., citing Sung H. Republic of Korea KR 9,707,148; 1997).

Flavonoids and terpenoids have antioxidative and antimicrobial properties, but they cannot be utilized as foodstuffs owing to their very low aqueous solubility and bitter taste. Sumiyoshi (1999) discussed the improvement of the properties of these plant components (flavonoids and terpenoids) with cyclodextrin complexation (Id., citing Sumiyoshi H. Nippon Shokuhin Shinsozai Kenkyukaishi 1999; 2: 109-14). CDs are used in the preparation of foodstuffs in different ways. For example, highly branched CDs are used in flour-based items like noodles, pie doughs, pizza sheets and rice cakes to impart elasticity and flexibility to dough (Id., citing Fujishima N, et al. Japanese Patent JP 136,898; 2001). They are also used in the preparation of antimicrobial food preservatives containing trans-2-hexanalin in apple juice preparation and in the processing of medicinal mushrooms for the preparation of crude drugs and health foods (Id., citing Takeshita K, Urata T. Japanese Patent JP 29,054; 2001). CDs are used in the preparation of controlled release powdered flavors and confectionery items and are also used in chewing gum to retain flavor for longer duration, a property highly valued by customers (Id., citing Mabuchi N, Ngoa M. Japanese Patent JP 128,638; 2001).

### Pharmaceuticals

A drug substance has to have a certain level of water solubility to be readily delivered to the cellular membrane, but it needs to be hydrophobic enough to cross the membrane. One of the unique properties of cyclodextrins is their ability to enhance drug delivery through biological membranes (Id.). The cyclodextrin molecules are relatively large (molecular weight ranging from almost 1000 to over 1500), with a hydrated outer surface, and under normal conditions, cyclodextrin molecules will only permeate biological membranes with considerable difficulty (Id., citing Frömming KH, Szejtli J. Cyclodextrins in pharmacy. Topics in inclusion science. Dordrecht: Kluwer Academic Publishers; 1994; Rajewski R A, Stella V J. J Pharm Sci 1996; 85: 1142-68). It is generally recognized that cyclodextrins act as true carriers by keeping the hydrophobic drug molecules in solution and delivering them to the surface of the biological membrane, e.g. skin, mucosa or the eye cornea, where they partition into the membrane. The relatively lipophilic membrane has a low affinity for the hydrophilic cyclodextrin molecules and therefore, they remain in the aqueous membrane exterior, e.g. the aqueous vehicle system (such as oil-in-water cream or hydrogel), salvia or the tear fluid. Conventional penetration enhancers, such as alcohols and fatty acids, disrupt the lipid layers of the biological barrier. Cyclodextrins, on the other hand, act as penetration enhancers by increasing drug availability at the surface of the biological barrier. For example, cyclodextrins have been used successfully in aqueous dermal formulations (Id., citing Uekama K, et al. J Pharm Pharmacol 1992; 44: 119-21), an aqueous mouthwash solution (Id., citing Kristmundsdóttir T, et al. Int J Pharm 1996; 139: 63-8), nasal drug delivery systems (Id., citing Kublik H, et al. Eur J Pharm Biopharm 1996; 42: 320-4), and several eye drop solutions (Id., citing Loftsson T, Stefánsson E. Drug Devel Ind Pharm 1997; 23: 473-81; van Dome H. Eur J Pharm Biopharm 1993; 39: 133-9; Jarho P, et al. Int J Pharm 1996; 137: 209-17).

The majority of pharmaceutical active agents do not have sufficient solubility in water, and traditional formulation systems for insoluble drugs involve a combination of organic solvents, surfactants, and extreme pH conditions, which often cause irritation or other adverse reactions. Cyclodextrins are not irritants and offer distinct advantages such as the stabilization of active compounds, reduction in volatility of drug molecules, and masking of malodors and bitter tastes (Id.).

There are numerous applications for cyclodextrins in the pharmaceuticals field. For example, the addition of α- or β-cyclodextrin increases the water solubility of several poorly water-soluble substances. In some cases this results in improved bioavailability, increasing the pharmacological effect, and allowing a reduction in the dose of the drug administered (Id.).

Inclusion complexes can also facilitate the handling of volatile products. This can lead to a different way of drug administering, e.g. in the form of tablets. Cyclodextrins are used to improve the stability of substances to increase their resistance to hydrolysis, oxidation, heat, light and metal salts. The inclusion of irritating products in cyclodextrins can also protect the gastric mucosa for the oral route, and reduce skin damage for the dermal route. Furthermore, cyclodextrins can be applied to reduce the effects of bitter or irritant tasting and bad smelling drugs (Id., citing Szetjli J. Chem Rev 1998; 98: 1743-53; Hedges R A. Chem Rev 1998; 98: 2035-44; Irie T, Uekama K. Adv Drug Deliv Rev 1999; 36: 101-23; Zhao T, et al. Antisense Res 1995; 5: 185-92).

Administered cyclodextrins are quite resistant to starch degrading enzymes, although they can be degraded at very low rates by α-amylases (Id.). α-Cyclodextrin is the slowest, and γ-cyclodextrin is the fastest degradable compound, due to their differences in size and flexibility. Degradation is not performed by saliva or pancreas amylases, but by α-amylases from microorganisms from the colon flora. Adsorption studies revealed that only 2-4% of cyclodextrins were adsorbed in the small intestines, and that the remainder is degraded and taken up as glucose. This can explain the low toxicity found upon oral administration of cyclodextrins (Id., citing Szetjli J. TIBTRCH 1989; 7: 171-4).

### Agricultural and Chemical Industries

Cyclodextrins form complexes with a wide variety of agricultural chemicals including herbicides, insecticides, fungicides, repellents, pheromones and growth regulators. Cyclodextrins can be applied to delay germination of seed. In grain treated with β-cyclodextrins some of the amylases that degrade the starch supplies of the seeds are inhibited. Initially the plant grows more slowly, but later on this is largely compensated by an improved plant growth yielding a 20-45% larger harvest (Id., citing Szetjli J. Chem Rev 1998; 98: 1743-53). Recent developments involve the expression of cyclodextrin glucanotransferases (CGTases) in plants (Id., citing Szetjli J. Chem Rev 1998; 98: 1743-53; Hedges R A. Chem Rev 1998; 98: 2035-44).

In the chemical industry, cyclodextrins are widely used to separate isomers and enantiomers, to catalyze reactions, to aid in various processes and to remove or detoxify waste materials. Cyclodextrins are widely used in the separation of enantiomers by high performance liquid chromatography (HPLC) or gas chromatography (GC). The stationary phases of these columns contain immobilized cyclodextrins or derived supra-molecular architectures. Other analytical applications can be found in spectroscopic analysis. In nuclear magnetic resonance (NMR) studies they can act as chiral shift agents and in Circular Dichroism as selective (chiral) agents altering spectra. In electrochemical chemistry they can be used to mask contaminating compounds, allowing more accurate determinations (Id., citing Szetjli J. Chem Rev 1998; 98: 1743-53).

One use of CDs in catalytic reactions is their ability to serve as enzyme mimics. These are formed by modifying naturally occurring CDs through substituting various functional compounds on the primary or secondary face of the molecule or by attaching reactive groups. These modified CDs are useful as enzyme mimics because of the molecular recognition phenomenon attributed to the substituted groups on the CD (Id., citing Szetjli J. Chem Rev 1998; 98: 1743-53). This ability results from binding of substrates in the hydrophobic cavity with the subsequent reaction initiated by catalytic groups linked to the CD. Rates of reaction are enhanced by almost 1000-fold by such modified CDs versus free solution due to the chelating effect of the CD catalysts. CDs can show enantiomeric specificity (meaning the degree to which one enantiomer (a molecule that is a mirror image of another) of a chiral product is preferentially produced in a chemical reaction) in such applications (Id., citing Villiers A. Compt Rendu 1891; 112: 536). The first chymotrypsin mimic was produced by modifying β-CD, which enhanced the rates of hydrolysis of activated esters and formation of amine bonds by 3.4-fold (Id., citing Ekberg B, et al. Carbohydr Res 1989; 192: 111-7; Morozumi T, et al. J Mol Catal 1991; 70: 399-406). Modified β-CD for the purpose of catalysis was used for the selective hydroxy-ethylation and hydroxymethylation of phenol. It was observed that chemical modification greatly promoted the catalytic activity, and the resulting CD derivative served as a transamine mimic, catalyzing the conversion of phenylpyruvic acid to phenylalanine. Atwood (1990) explained the use of modified α-cyclodextrin in the reduction of Mn(III) porphyrin (Id., citing Atwood J L. Inclusion phenomenon and molecular recognition. New York: Plenum; 1990).

Due to their steric (meaning spatial arrangement) effects, CDs also play a significant role in biocatalytic processes by increasing the enantioselectivity. After the formation of inclusion complex with the prochiral guest molecule, the preferential attack by the reagent takes place only from one of the enantioselective faces, resulting in higher enantioselectivity. For example, it was reported by Kamal et al. (1991) that the hydrolysis of racemic arylpropionic esters by bovine serum albumin, a carrier protein, resulted in low enantioselectivity (50-81% ee), while addition of β-CD to this reaction not only enhanced the enantioselectivity (80-99% ee) but also accelerated the rate of hydrolysis (Id., citing Kamal A, et al. Tetrahedron: Asymmetry 1991; 2: 39). Rao et al. (1990) demonstrated that chiral recognition during cycloaddition reaction of nitriloxides or amines to the C≡C triple bond using baker's yeast as a chiral catalyst was improved by the addition of CDs, increasing the enantioselectivity of yeast by up to 70% (Id., citing Rao K R, et al. Tetrahedron Letters 1990; 31: 892-9).

Cyclodextrins can play a major role in environmental science in terms of solubilization of organic contaminants, enrichment and removal of organic pollutants and heavy metals from soil, water and atmosphere (Id., citing Gao S, Wang L. Huanjing Kexue Jinzhan 1998; 6: 80-6). For example, CDs are applied in water treatment to increase the stabilizing action, encapsulation and adsorption of contaminants (Id., citing Wu C, Fan J. Shuichuli Jishu 1998; 24: 67-70). Using cyclodextrins, highly toxic substances can be removed from industrial effluent by inclusion complex formation. In the mother liquor of the insecticide trichlorfon, the uncrystallizable trichlorfon can be converted into a β-CD complex and in a single treatment 90% of the toxic material is removed (Id., citing Szetjli J. Chem Rev 1998; 98: 1743-53; Hedges R A. Chem Rev 1998; 98: 2035-44). Wastewaters containing environmentally unacceptable aromatic compounds such as phenol, p-chlorophenol and benzene after treating with β-CD have considerably reduced levels of these aromatic hydrocarbons from their initial levels. Cyclodextrins are used to scrub gaseous effluent from organic chemical industries (Id., citing Szetjli J. Chem Rev 1998; 98: 1743-53; Hedges R A. Chem Rev 1998; 98: 2035-44). Solubility enhancement phenomenon of CDs is used for testing of soil remediation. Reid et al. (1999) discussed the soil test for determining bioavailability of pollutants using CD and its derivatives (Id., citing Reid B J, et al. PCT Int Appl WO 99 54,727; 1999). CD complexation also resulted in the increase of water solubility of three benzimidazole-type fungicides (thiabendazole, carbendazim and fuberidazole) making them more available to soil. In addition to its ability to increase the solubility of the hydrocarbon for biodegradation and bioremediation, CDs also decrease the toxicity resulting in an increase in microbial and plant growth. β-Cyclodextrins accelerated the degradation of all types of hydrocarbons influencing the growth kinetics, producing higher biomass yield and better utilization of hydrocarbon as a carbon and energy source. The low cost, biocompatible and effective degradation makes β-cyclodextrins a useful tool for bioremediation process (Id., citing Bardi L, et al. Enzyme Microb Technol 2000; 27: 709-13).

### Adhesives, Coatings and Other Polymers

Cyclodextrins increase the tackiness and adhesion of some hot melts and adhesives. They also make additives and blowing agents compatible with hot melt systems. The interaction between polymer molecules in associative thickening emulsion-type coatings such as paints tends to increase viscosity, and CDS can be used to counteract this undesirable effect (Id.).

Notwithstanding the foregoing, the effect of inclusion of a guest molecule in a cyclodextrin host remains unpredictable. For example, although various cyclodextrin complexes have been reported to enhance the bioavailability of small molecule drugs, cyclodextrin inclusion complexes have also been reported to have either no effect on host bioavailability or to in fact decrease the bioavailability of certain guest compounds (Carrier R L, et al. J Control Release. 2007 Nov. 6; 123(2): 78-99). The interaction of cyclodextrins with labile compounds can also result in several outcomes: cyclodextrins can retard degradation, can have no effect on reactivity, or can accelerate drug degradation (Loftsson T, Brewster M E. J Pharm Sci. 1996 October; 85(10): 1017-25). In addition, the unpredictability of thermodynamic quantities related to inclusion complex formation have also been reported (Steffen A, Apostolakis J. Chem Cent J. 2007 Nov. 15; 1: 29).

The described invention provides improved β-Cyclodextrin inclusion complexes, methods of making the inclusion complexes, and pharmaceutical and cosmetic compositions containing the inclusion complexes.

### SUMMARY OF THE INVENTION

According to one aspect, the described invention provides a method for improving incorporation of a guest compound in a cavity of a hydroxypropyl-β-cyclodextrin host comprising: (a) establishing a vacuum in the cavity of the hydroxypropyl-β-cyclodextrin (HPBCD); (b) adding the guest compound, wherein the guest compound is substantially free of a solvent; (c) incorporating the guest compound into the cavity; and (d) forming an active agent-hydroxypropyl-β-cyclodextrin inclusion complex. According to some embodiments, the solvent is an aqueous solvent or an organic solvent.

According to one embodiment of the method, the guest compound may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% included into the cavity of the cyclodextrin molecule. According to another embodiment, a molar ratio of the guest compound to the HPBCD may be about 10:1, about 9:1, about 8:1, about 7:1, about 6:1, about 5:1, about 4:1, about 3:1, about 2:1, about 1:1 to about 1:300; i.e., about 1:1, about 1:2, about 1:3, about 1:4, about 1:5, about 1:6, about 1:7, about 1:8, about 1:9, about 1:10, about 1:11, about 1:12, about 1:13, about 1:14: about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1: 84, about 1:85, about 1:86, about 1:87, about 1:88, about 1:89, about 1:90, about 1:91, about 1:92, about 1:93, about 1:94, about 1:95, about 1:96, about 1:97, about 1: 98, about 1:99, about 1:100. According to another embodiment, the guest compound is a lipophilic active agent. According to another embodiment, the guest compound is selected from the group consisting of an anti-fungal agent, an anti-histamine agent; an anti-hypertensive agent; an anti-protozoal agent; an anti-oxidant; an anti-pruritic agent; an anti-skin atrophy agent; an anti-viral agent; a caustic agent; a calcium channel blocker; a cytokine-modulating agent; a prostaglandin analog; a chemotherapeutic agent; an irritant agent; a TRPC channel inhibitor agent; and a vitamin.

According to another embodiment, the method further comprises combining a therapeutic amount of the active agent-inclusion complex with a pharmaceutically acceptable carrier; and forming a pharmaceutical composition. According to another embodiment, the pharmaceutical composition is effective (a) to reduce contact-based side effects compared to the active agent alone; or (b) to improve bioavailability when compared to the bioavailability of the non-complexed active agent; or (c) to improve stability of the active agent when compared to the stability of the non-complexed active agent alone; or (d) to improve penetration of the active agent when compared to the penetration of the non-complexed active agent alone; (e) to improve retention of the active agent in a targeted tissue when compared to the retention of the noncomplexed active agent alone; or (f) to reduce toxicity of the active agent when compared to the toxicity of the non-complexed active agent alone; or (g) to deliver a minimal effective concentration of the active agent to locations in vivo with a small amount of formulation volume. According to another embodiment, the method further comprises formulating the pharmaceutical composition with a polymer, wherein the composition is characterized by slow release; or wherein the composition is characterized by controlled release; or wherein the composition is characterized by sustained release.

According to another embodiment, the method further comprises combining a cosmetic amount of the active agent-inclusion complex with a cosmetically acceptable carrier; and forming a cosmetic composition. According to another embodiment, the cosmetic composition is effective (a) to reduce contact-based side effects compared to the active agent alone; or (b) to improve bioavailability when compared to the bioavailability of the non-complexed active agent; or (c) to improve stability of the active agent when compared to the stability of the non-complexed active agent alone; or (d) to improve penetration of the active agent when compared to the penetration of the non-complexed active agent alone; (e) to improve retention of the active agent in a targeted tissue when compared to the retention of the noncomplexed active agent alone; or (f) to reduce toxicity of the active agent when compared to the toxicity of the non-complexed active agent alone; or (g) to deliver a minimal effective concentration of the active agent to locations in vivo with a small amount of formulation volume. According to another embodiment, the method further comprises formulating the cosmetic composition with a polymer, wherein the composition is characterized by slow release; or wherein the composition is characterized by controlled release; or wherein the composition is characterized by sustained release. According to some embodiments, the method further comprises causing the active agent-hydroxypropylβcyclodextrin inclusion complex to form a dendrimer.

These and other advantages of the invention will be apparent to those of ordinary skill in the art by reference to the following detailed description and the accompanying drawings.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows an illustration of the anatomy of human skin. From Mayo Foundation for Medical Education and Research.

FIG. 2 shows the layers of the epidermis below the stratum corneum, including the stratum lucidum, stratum granulosum, stratum germinativum, and stratum basale.

UV-Vis was used for identification and quantification of active agents and degradation products. As shown in FIG. 3A, Benzocaine displays peak maximums at 272 nm and 296 nm. The HPBCD benzocaine complex exhibits peak maximums at 260 nm, 290 nm, and 310 nm. HPBCD has a small broad peak at 241 nm. As shown in FIG. 3B, CBD displays peak maximums at 221 nm, 233 nm, 239 nm and 278 nm. The HPBCD CBD complex exhibits peak maximums at 221 nm, 227 nm, 233 nm and 278 nm. HPBCD has a small broad peak at 241 nm. As shown in FIG. 3C, Minoxidil displays peak maximums at 230 nm, 250 nm, 260 nm, 280 nm and 290 nm. The HPBCD minoxidil complex exhibits peak maximums at 255 nm and 280 nm. HPBCD has a small broad peak at 241 nm. As shown in FIG. 3D, Niacinamide displays peak maximums at 235 nm and 255 nm. The HPBCD niacinamide complex exhibits peak maximums at 240 nm, 265 nm, and 295 nm. HPBCD has a small broad peak at 241 nm. This shows the cyclodextrin molecule does not interfere in the prominent active region of niacinamide, thus UV can be used for analysis of the complex. As shown in FIG. 3E, Pycnogenol displays peak maximums at 230 nm, 280 nm and 310 nm. The HPBCD pycnogenol complex exhibits peak maximums at 225 nm, 240 nm, 275 nm and 305 nm. HPBCD has a small broad peak at 241 nm. As shown in FIG. 3F, Tamanu oil displays peak maximums at 215 nm, 269 nm and 296 nm. The HPBCD tamanu oil complex exhibits peak maximums at 206 nm, 212 nm, 218 nm, 262 nm and 366 nm. HPBCD has a small broad peak at 241 nm. As shown in FIG. 3G, Tetrahydrocurcumin displays peak maximums at 209 nm, 218 nm and 278 nm. The HPBCD tetrahydrocurcumin complex exhibits peak maximums at 225 nm and 280 nm. HPBCD has a small broad peak at 241 nm.

FIG. 4 shows overlaid differential scanning calorimetry (DSC) curves for niacinamide (green), with a single melting peak at about 135° C.; HPBCD (red) with a broad melting curve that peaks at about 100° C., and HPBCD niacinamide inclusion complex (blue), with no niacinamide melting peak present, but a broad melting curve that peaks at around 100° C.

FIG. 5 shows overlaid differential scanning calorimetry (DSC) curves for Tamanu oil, which has no discernable melting peak (red), HPBCD (green) with a melting peak at about 106° C.; and HPBCD tamanu inclusion complex (blue), with a melting peak at about 110° C.

FIG. 6 shows overlaid differential scanning calorimetry (DSC) curves for crystalline cannabidiol (CBD) (green) with a sharp melting peak at about 65° C.; a melting curve for HPBCD (red) with a minimum of about 106° C., and for HPBCD-CBD inclusion complex (blue), with a broad melting peak at about 110° C. In the spectrum of the complex, a smaller melting peak is observed, which corresponds to the portion of the CBD molecule hanging outside the cyclodextrin cavity, and is shifted to around 60° C., due to steric hindrance.

FIG. 7 shows overlaid differential scanning calorimetry (DSC) curves for tetrahydrocurcumin (green) with a single melting peak at about 106° C.; HPBCD with a broad melting curve (red) with a minimum at about 104° C.; and HPBCD tetrahydrocurcumin inclusion complex (blue), with a broad melting curve that peaks at about 110° C. There is a small melting peak around 88° C., which corresponds to the portion of the tetrahydrocurcumin that is hanging outside the cyclodextrin cavity.

FIG. 8 shows overlaid DSC curves for benzocaine (green), HPBCD (blue) and HPBCD-benzocaine inclusion complex.

FIG. 9 shows overlaid DSC curves for minoxidil (red), HPBCD (green), and HPBCD-minoxidil inclusion complex (blue).

FIG. 10 shows overlaid DSC curves for pycnogenol (green), HPBCD (blue), and HPBCD-pycogenol complex (red).

FIG. 11A shows dissolution profiles of HPBCD benzocaine complex using the compound as a dry granulation; a slightly higher percentage of the active was dissolved at higher pH value. The dissolution profile displays a burst like, zero-order release. A zero-order release implies the active release is independent of the initial drug concentration. FIG. 11B shows a concentration curve of the complex. The wavelength for analysis of HPBCD benzocaine complex was 290 nm

FIG. 12A shows dissolution profiles of HPBCD CBD complex using the compound as a dry granulation. A slightly higher percentage of the active was dissolved at higher pH value. The dissolution profile adopts the characteristic shape of a sustained release profile. Sustained release implies the drug is released over a longer period of time, with the percentage decreasing slightly over time. This type of profile can also be considered as zero-order. FIG. 12B shows a concentration curve of the complex. The wavelength for analysis of HPBCD CBD complex was 233 nm.

FIG. 13A shows dissolution profiles of HPBCD minoxidil complex using the compound as a dry granulation. A substantially higher percentage of the active was dissolved at lower pH value. The dissolution profile displays a burst like, zero-order release. FIG. 13B shows a concentration curve of the complex the wavelength for analysis of HPBCD minoxidil complex was 280 nm.

FIG. 14A shows dissolution profiles of HPBCD niacinamide complex using the compound as a dry granulation. A higher percentage of the active was dissolved at lower pH value. The dissolution profile displays a burst like, zero-order release. FIG. 14B shows a concentration curve of the complex. The wavelength for analysis of HPBCD niacinamide complex was 265 nm.

FIG. 15A shows dissolution profiles of HPBCD pycnogenol complex using the compound as a dry granulation. The percentage of the active dissolved was virtually the same at lower and higher pH value. The dissolution profile displays a burst like, zero-order release. FIG. 15B shows a concentration curve of the complex. The wavelength for analysis of HPBCD pycnogenol complex was 225 nm.

FIG. 16A shows the dissolution profiles of HPBCD tamanu oil complex using the compound as a dry granulation. A higher percentage of the active was dissolved at higher pH value. The dissolution profile adopts the characteristic shape of a sustained release profile. Sustained release implies the drug is released over a longer period of time, with the percentage decreasing slightly over time. This type of profile can also be considered as zero-order. FIG. 16B shows a concentration curve of the complex. The wavelength for analysis of HPBCD tamanu oil complex was 212 nm.

FIG. 17A shows the dissolution profiles of HPBCD tetrahydrocurcumin complex using the compound as a dry granulation. The percentage of the active dissolved was similar at lower and higher pH value. At lower pH, the percentage of active dissolved decreases somewhat over time, resembling a sustained release profile. The dissolution profile displays a burst like, zero-order release. A zero-order release indicates the active release is independent of the initial drug concentration. FIG. 17B shows a concentration curve of the complex. The wavelength for analysis of HPBCD tetrahydrocurcumin complex was 225 nm.

FIG. 18 is an A_{L} type phase solubility diagram for components S and L. A linear increase in the solubility of S is classified as AL type by Higuchi and Connors [Phase-solubility techniques, Adv. Anal. Chem. Instr. 4, 117-122, (1965)] and demonstrates that the solubility of S is increased by the presence of L. Type A diagrams indicate the formation of a soluble complex between S and L. If the slope of an A_{L} type diagram is greater than unity, then at least one component has a concentration that is greater than one. A slope of less than unity indicates a 1:1 stoichiometry between components S and L.

FIG. 19 is the phase solubility diagram of HP-B-CD and Niacinamide. It shows a linear increase in solubility and is classified as A_{L} type by the Higuchi and Connors classification. This demonstrates the formation of a soluble complex between HPBCD and niacinamide. The slope of the graph is less than one (slope=4.44×10⁻¹) which indicates a 1:1 stoichiometry of the complex. The association constant (Kc) for complex formation was found to be 79.856×10⁻² M⁻¹ and was calculated using Eq. (1). The absorbance was measured by UV at λ=217 nm.

FIG. 20 is the phase solubility diagram of HPBCD and CBD. It shows a linear increase in solubility and is classified as AL type by the Higuchi and Connors classification. This demonstrates the formation of a soluble complex between HPBCD and CBD. The slope of the graph is less than one (slope=2.97×10⁻¹) which indicates a 1:1 stoichiometry of the complex. The association constant (Kc) for complex formation was found to be 42.247×10⁻² M⁻¹ and was calculated using Eq. (1). The absorbance was measured by UV at λ=280 nm.

FIG. 21 is the phase solubility diagram of HPBCD and pycnogenol. It shows a linear increase in solubility and is classified as A_{L} type by the Higuchi and Connors classification. This demonstrates the formation of a soluble complex between HPBCD and pycnogenol. The slope of the graph is greater than one (slope=15.87×10⁻¹) which indicates that the stoichiometry of the complex is not 1:1. The association constant (Kc) for complex formation was found to be 270.358×10⁻²M⁻¹ and was calculated using Eq. (1). The absorbance was measured by UV at λ=280 nm.

FIG. 22 is the phase solubility diagram of HPBCD and tetrahydrocurcumin. It shows a linear increase in solubility and is classified as AL type by the Higuchi and Connors classification. This demonstrates the formation of a soluble complex between HPBCD and tetrahydrocurcumin. The slope of the graph is greater than one (slope=12.84×10⁻¹) which indicates that the stoichiometry of the complex is not 1:1. The association constant (Kc) for complex formation was found to be 452.113×10⁻² M⁻¹ and was calculated using Eq. (1). The absorbance was measured by UV at λ=280 nm.

FIG. 23 is the phase solubility diagram of HPBCD and tamanu oil. This diagram shows a linear increase in solubility and is classified as AL type by the Higuchi and Connors classification. This demonstrates the formation of a soluble complex between HPBCD and tamanu oil. The slope of the graph is greater than one (slope=14.83×10⁻¹) which indicates that the stoichiometry of the complex is not 1:1. The association constant (Kc) for complex formation was found to be 307.039×10⁻² M⁻¹ and was calculated using Eq. (1). The absorbance was measured by UV at λ=266 nm.

FIG. 24 is the phase solubility diagram of HPBCD and minoxidil. This diagram shows an initial linear increase in solubility followed by the formation of a plateau. The plateau indicates complete solubilization of minoxidil that additional amounts of HPBCD does not alter. This diagram is still considered as A type by the Higuchi and Connors classification. Since the graph is not linear, the slope does not give an accurate indication of the stoichiometry. The slope of the linear part of the graph was used to calculate the association constant (slope=11.249). The association constant (Kc) for complex formation was found to be 109.757×10⁻² M⁻¹ and was calculated using Eq. (1). The absorbance was measured by UV at λ=290 nm.

FIG. 25 is the phase solubility diagram of HPBCD and benzocaine. This diagram shows an initial linear increase in solubility followed by the formation of a plateau. The plateau indicates complete solubilization of benzocaine that additional amounts of HPBCD does not alter. This diagram is still considered as A type by the Higuchi and Connors classification. Since the graph is not linear, the slope does not give an accurate indication of the stoichiometry. The slope of the linear part of the graph was used to calculate the association constant (slope=33.256). The association constant (Kc) for complex formation was found to be 103.100×10⁻² M⁻¹ and was calculated using Eq. (1). The absorbance was measured by UV at λ=305 nm.

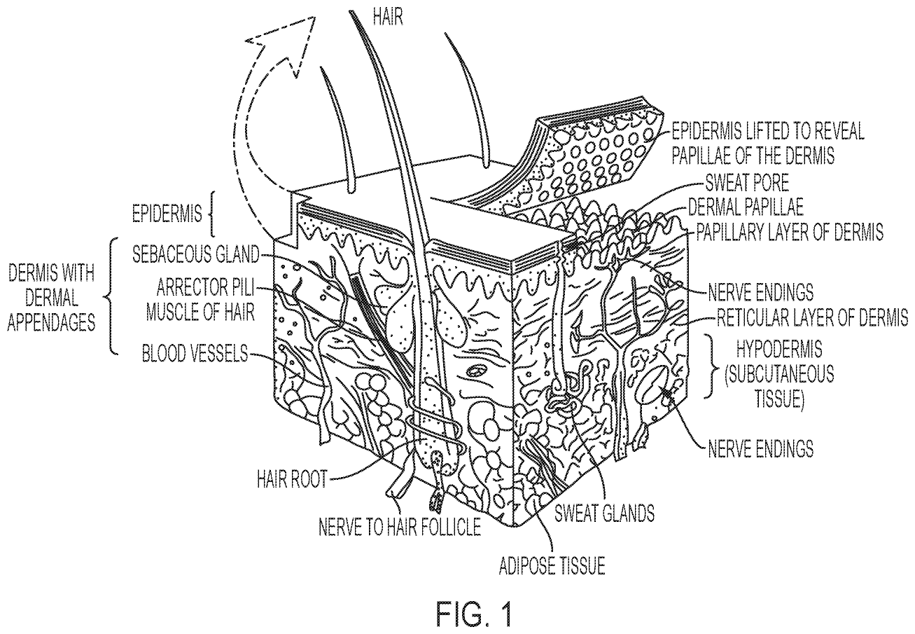

FIG. 26 shows a standard graph of concentration versus time for a zero order kinetic reaction to determine the rate of reaction (k). The degradation kinetics of a zero-order reaction does not depend on the concentration of the reagents. Therefore, the rate of reaction (k)=−d[C]/dt, where [C] indicates decreasing concentration of reagent and t indicates time. Integration of the rate equation between initial concentration at time t=0 (C0) and concentration after time t=t (Ct) yields the equation Ct=C0−kt. When this linear equation is plotted according to FIG. 1, with concentration on the x vertical axis and time on the y horizontal axis, the slope of the graph is equal to −k.

FIG. 27 shows the degradation graph of concentration versus time for HPBCD pycnogenol solution in deionized water at 25° C. It shows a zero-order kinetic reaction in the presence of three molar concentrations of phosphoric acid.

FIG. 28 shows the degradation graph of concentration versus time for HPBCD niacinamide solution in deionized water at 25° C. It shows a zero-order kinetic reaction in the presence of three molar concentrations of phosphoric acid.

FIG. 29 shows the degradation graph of concentration versus time for HPBCD tamanu oil solution in deionized water at 25° C. It shows a zero-order kinetic reaction in the presence of three molar concentrations of phosphoric acid.

FIG. 30 shows the degradation graph of concentration versus time for HPBCD tetrahydrocurcumin solution in deionized water at 25° C. It shows a zero-order kinetic reaction in the presence of three molar concentrations of phosphoric acid.

FIG. 31 shows the degradation graph of concentration versus time for HPBCD minoxidil solution in deionized water at 25° C. It shows a zero-order kinetic reaction in the presence of three molar concentrations of phosphoric acid.

FIG. 32 shows the degradation graph of concentration versus time for HPBCD benzocaine solution in deionized water at 25° C. It shows a zero-order kinetic reaction in the presence of three molar concentrations of phosphoric acid.

FIG. 33 shows the degradation graph of concentration versus time for HPBCD CBD solution in deionized water at 25° C. It shows a zero-order kinetic reaction in the presence of three molar concentrations of phosphoric acid.

FIG. 34 is an FTIR spectrum of HPBCD. The region from 700-1200 cm-1 shows peaks due to the C—O—C bending, C≡C—O stretching, and skeletal vibration involving the α-1,4 linkage. The region from 1200-1500 cm⁻¹ shows peaks due to C—H and O—H bending. The small broad peak at 1650 cm⁻¹ is the H—O—H bending peak due to water of crystallization of water molecules trapped within the cavity of the cyclodextrin molecule. The region of 2850-3000 cm⁻¹ is the C—H stretch and the strong broad peak at 3300 cm⁻¹ is the O—H stretch.

FIG. 35 shows overlaid FTIR spectra for benzocaine (red), HPBCD (green), and HPBCD benzocaine inclusion complex (blue). The spectrum of the inclusion complex mirrors the spectrum of HPBCD, which indicates that the benzocaine molecule entered the cavity of the cyclodextrin. The N—H amine group stretching peaks in the 3200-3500 cm⁻¹ region of benzocaine disappeared, as well as the aromatic peaks from the benzene ring (3000 cm⁻¹ and 1300-1500 cm⁻¹), indicating insertion of this portion of the molecule within the HPBCD cavity.

FIG. 36 shows overlaid FTIR spectra for CBD (red), HPBCD (green), and HPBCD CBD inclusion complex (blue). A sizeable portion of the CBD molecule hangs outside the cyclodextrin cavity. The region from 700-1200 cm⁻¹ shows peaks due to the C—O—C bending, C≡C—O stretching, and skeletal vibration involving the α-1,4 linkage of HPBCD, and the spectra of the complex mirrors this region. The 1:1 molar ratio of HPBCD to CBD only allows one ring of the CBD molecule to enter the cyclodextrin cavity, thus there is a large portion of the CBD molecule hanging outside the HPBCD.

FIG. 37 shows overlaid FTIR spectra for minoxidil (green), HPBCD (blue), and HPBCD minoxidil inclusion complex (red). The spectrum of the inclusion complex mirrors the spectrum of HPBCD and indicates that the minoxidil molecule is fully incorporated into the cavity of the cyclodextrin. The aromatic peaks from the aminopyrimidine and piperidine rings (1200-1700 cm⁻¹) of minoxidil are absent from the spectrum of the complex, indicating insertion within the HPBCD cavity. The 2:1 molar ratio of HPBCD to minoxidil allows both rings of the minoxidil molecule to be incorporated into two molecules of HPBCD, thus none of the minoxidil molecule is outside the cyclodextrin cavity. The small broad peak at 1650 cm⁻¹ (H—O—H bending) is the water of crystallization peak and indicates that there are a few water molecules trapped within the cavity of the HPBCD minoxidil complex. The absence of new peaks in the spectrum of the inclusion complex indicates a non-covalent interaction between the host and guest molecule.

FIG. 38 shows overlaid FTIR spectra for niacinamide (green), HPBCD (blue), and HPBCD niacinamide inclusion complex (red). The spectrum of the inclusion complex mirrors the spectrum of HPBCD, which indicates that the niacinamide molecule entered the cavity of the cyclodextrin moiety. The aromatic peaks from the pyridine ring (1200-1500 cm⁻¹) are absent from the spectrum of the complex, indicating insertion of this portion of the molecule within the HPBCD cavity. The peaks from the complex spectra at 1695 cm-1 (C═O stretch), 1610 cm⁻¹ (N—H bend) and 1600 cm⁻¹ (N—H bend) correspond to the amide portion of the niacinamide molecule which is outside the cyclodextrin cavity.

FIG. 39 shows overlaid FTIR spectra for pycnogenol (green), HPBCD (blue), and HPBCD pycnogenol inclusion complex (red). The spectrum of the inclusion complex mirrors the spectrum of HPBCD, which indicates that the pycnogenol molecule entered the cavity of the cyclodextrin. The 3:1 molar ratio of HPBCD to pycnogenol allows three of the rings of the procyanidin or proanthocyanidin molecule to be incorporated within the cavity of three cyclodextrin molecules. The fourth ring from the procyanidin and proanthocyanidin moieties of pycnogenol lies outside the cavity of HPBCD.

FIG. 40 shows overlaid FTIR spectra for tamanu oil (green), HPBCD (blue), and HPBCD tamanu oil inclusion complex (red). The spectrum of the inclusion complex mirrors the spectrum of HPBCD, which indicates that the tamanu oil entered the cavity of the cyclodextrin. Tamanu oil is made up of the C16 and C18 fatty acids oleic, linoleic, palmitic and stearic. The 3:1 molar ratio of HPBCD to tamanu oil allows for most of the fatty acid carbon chains to be incorporated within the cyclodextrin cavity. The peaks from the complex spectra at 2915 cm⁻¹ (C—H stretch) and 2865 cm⁻¹ (C—H stretch) are asymmetrical stretching vibrations of the —CH2 bonds from the portion of the fatty acid hanging outside the cavity of HPBCD. The carboxylic acid headgroup of the fatty acid also lies outside the cyclodextrin cavity, with the carbonyl peak in the complex spectra occurring at 1750 cm⁻¹ (C═O stretch).

FIG. 41 shows overlaid FTIR spectra for tetrahydrocurcumin (green), HPBCD (blue), and HPBCD tetrahydrocurcumin inclusion complex (red). The spectrum of the inclusion complex mirrors the spectrum of HPBCD, which indicates that the tetrahydrocurcumin molecule entered the cavity of the cyclodextrin. The aromatic peaks from the benzene rings (1100-1400 cm⁻¹) and the strong carbonyl peak (1600 cm⁻¹) are absent from the spectrum of the complex, indicating insertion of these portions of the molecule within the HPBCD cavity. The 3:1 molar ratio of HPBCD to tetrahydrocurcumin allows both rings of the tetrahydrocurcumin molecule, as well as the carbonyl groups to be incorporated into three molecules of HPBCD.

FIG. 42 shows representative HPLC chromatographs of calibration standards for niacinamide. The y-axis of each chromatogram is a measure of the intensity of absorbance (in units of mAU, or milli-Absorbance Units). The x-axis is in units of time (minutes), and is used to determine the retention time (tR) for each peak.

FIG. 43 shows representative chromatographs of calibration standards for tamanu oil. The main peak is for oleic acid. The y-axis of each chromatogram is a measure of the intensity of absorbance (in units of mAU, or milli-Absorbance Units). The x-axis is in units of time (minutes), and is used to determine the retention time (tR) for each peak.

FIG. 44 shows representative chromatographs of calibration standards for tetrahydrocurcumin (TC). The y-axis of each chromatogram is a measure of the intensity of absorbance (in units of mAU, or milli-Absorbance Units). The x-axis is in units of time (minutes), and is used to determine the retention time (tR) for each peak.

FIG. 45 shows representative chromatographs of calibration standards for cannabidiol (CBD). The y-axis of each chromatogram is a measure of the intensity of absorbance (in units of mAU, or milli-Absorbance Units). The x-axis is in units of time (minutes), and is used to determine the retention time (tR) for each peak.

FIG. 46A is a transdermal bar graph, which is a plot of delivered dose (in μg/cm²) versus time elapsed (in hours) for Nourishing Cream containing either Niacinamide (molecular weight, 122.127 g/mol) or a Niacinamide HBPCD inclusion complex. FIG. 46B is a flux bar graph, which is a plot of flux versus time elapsed (hours), for Nourishing Cream containing either Niacinamide (molecular weight, 122.127 g/mol) or a Niacinamide HBPCD inclusion complex. Flux, with values in μg/cm²/hr, is obtained by dividing the delivered dose by the amount of time (either 8, 24, or 48 hours). FIG. 46C is a skin retention bar graph, which is a plot of delivered dose (μg/cm²) versus time (hrs). It shows the amount of active in the epidermis and the dermis after 48 hours (in μg/cm²) for Nourishing Cream containing either Niacinamide (molecular weight, 122.127 g/mol) or a Niacinamide HBPCD inclusion complex.

FIG. 47A is a transdermal bar graph, which is a plot of delivered dose (in μg/cm²) versus time elapsed (in hours) for Pain Relief Cream containing either Cannabidiol (“CBD”, molecular weight 314.464 g/mol) or a Cannabidiol-HBPCD inclusion complex. FIG. 47B is a flux bar graph, which is a plot of flux versus time elapsed (hours), for Pain Relief Cream containing either Cannabidiol (“CBD”, molecular weight 314.464 g/mol) or a Cannabidiol-HBPCD inclusion complex. Flux, with values in μg/cm2/hr, is obtained by dividing the delivered dose by the amount of time (either 8, 24, or 48 hours). FIG. 47C is a skin retention bar graph, which is a plot of delivered dose (μg/cm²) versus time (hrs). It shows the amount of active in the epidermis and the dermis after 48 hours (μg/cm²) for Pain Relief Cream containing either Cannabidiol (“CBD”, molecular weight 314.464 g/mol) or a Cannabidiol-HBPCD inclusion complex.

FIG. 48A is a transdermal bar graph, which is a plot of delivered dose (in μg/cm²) versus time elapsed (in hours) for Scar Reduction Cream containing either Tamanu oil or a tamanu oil-HBCD complex. Because oleic acid (molecular weight 282.417 g/mol) is the main constituent of tamanu oil, it was selected for analysis. FIG. 48B is a flux bar graph, which is a plot of flux versus time elapsed (hours), for Scar Reduction Cream containing either Tamanu oil or a tamanu oil-HBCD complex. Because oleic acid (molecular weight 282.417 g/mol) is the main constituent of tamanu oil, it was selected for analysis. Flux, with values in μg/cm²/hr, is obtained by dividing the delivered dose by the amount of time (either 8, 24, or 48 hours). FIG. 48C is a skin retention bar graph, which is a plot of delivered dose (μg/cm²) versus time (hrs). It shows the amount of active in the epidermis and the dermis after 48 hours (in μg/cm2) for Scar Reduction Cream containing either Tamanu oil or a tamanu oil-HBCD complex. Because oleic acid (molecular weight 282.417 g/mol) is the main constituent of tamanu oil, it was selected for analysis.

FIG. 49A is a transdermal bar graph, which is a plot of delivered dose (in μg/cm²) versus time elapsed (in hours) for Brightening Cream containing either tetrahydrocurcumin (“TC”, molecular weight, 372.417 g/mol) or a tetrahydrocurcumin-HBPCD inclusion complex. FIG. 49B is a flux bar graph, which is a plot of flux versus time elapsed (hours), for Brightening Cream containing either tetrahydrocurcumin (“TC”, molecular weight, 372.417 g/mol) or a tetrahydrocurcumin-HBPCD inclusion complex. Flux, with values in μg/cm²/hr, is obtained by dividing the delivered dose by the amount of time (either 8, 24, or 48 hours). FIG. 49C is a skin retention bar graph, which is a plot of delivered dose (μg/cm²) versus time (hrs). It shows the amount of active in the epidermis and the dermis after 48 hours (in μg/cm2) for Brightening Cream containing either tetrahydrocurcumin (“TC”, molecular weight, 372.417 g/mol) or a tetrahydrocurcumin-HBPCD inclusion complex.

### DETAILED DESCRIPTION OF EMBODIMENTS

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, for example, about 50% means in the range of 40%-60%, inclusive, i.e., 40%, 41%, 42%, 43%, 44%, 45%, 46%, 47%, 48%, 49%, or 50%.

The term “active” refers to the ingredient, component or constituent of the compositions of the described invention responsible for the intended cosmetic or therapeutic effect.

“Administering” when used in conjunction with a therapeutic means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with CDs or compositions thereof, can include, but is not limited to, providing CDs into or onto the target organ, tissue or cell; or providing CDs systemically to a patient by, e.g., intravenous injection, whereby the therapeutic reaches the target organ, tissue or cell. “Administering” may be accomplished by parenteral, oral or topical administration, by inhalation, or by such methods in combination with other known techniques.

The terms “animal,” “patient,” and “subject” as used herein include, but are not limited to, humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals.

As used herein, the phrase “subject in need” of treatment for a particular condition is a subject having that condition, diagnosed as having that condition, or at risk of developing that condition. According to some embodiments, the phrase “subject in need” of such treatment also is used to refer to a patient who (i) will be administered a composition of the described invention; (ii) is receiving a composition of the described invention; or (iii) has received at least one a composition of the described invention, unless the context and usage of the phrase indicates otherwise.

The term “aqueous” is to be understood in the meaning that the pharmaceutical composition contains water as a solvent, whereby also one or more additional solvents may be optionally present.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “bioavailability” and its various grammatical forms as used herein mean the rate and extent to which an active ingredient or active moiety becomes available at the site of action in vivo. Bioavailability/bioequivalence may be demonstrated by several in vivo and in vitro methods. The selection of the method used to meet an in vivo or in vitro testing requirement depends upon the purpose of the study, the analytical methods available, and the nature of the drug product. The method used must be capable of measuring bioavailability or establishing bioequivalence, as appropriate, for the product being tested.

The following in vivo and in vitro approaches, in descending order of accuracy, sensitivity, and reproducibility, are considered acceptable for determining the bio availability or bioequivalence of a drug product. (1)(i) An in vivo test in humans in which the concentration of the active ingredient or active moiety, and, when appropriate, its active metabolite(s), in whole blood, plasma, serum, or other appropriate biological fluid is measured as a function of time. This approach is particularly applicable to dosage forms intended to deliver the active moiety to the bloodstream for systemic distribution within the body; or (ii) An in vitro test that has been correlated with and is predictive of human in vivo bioavailability data; or (2) An in vivo test in humans in which the urinary excretion of the active moiety, and, when appropriate, its active metabolite(s), are measured as a function of time. The intervals at which measurements are taken should ordinarily be as short as possible so that the measure of the rate of elimination is as accurate as possible. Depending on the nature of the drug product, this approach may be applicable to the category of dosage forms described in paragraph (1)(i). This method is not appropriate where urinary excretion is not a significant mechanism of elimination. (3) An in vivo test in humans in which an appropriate acute pharmacological effect of the active moiety, and, when appropriate, its active metabolite(s), are measured as a function of time if such effect can be measured with sufficient accuracy, sensitivity, and reproducibility. This approach is applicable to the category of dosage forms described in paragraph (1)(i) only when appropriate methods are not available for measurement of the concentration of the moiety, and, when appropriate, its active metabolite(s), in biological fluids or excretory products but a method is available for the measurement of an appropriate acute pharmacological effect. This approach may be particularly applicable to dosage forms that are not intended to deliver the active moiety to the bloodstream for systemic distribution. (4) Well-controlled clinical trials that establish the safety and effectiveness of the drug product, for purposes of measuring bioavailability, or appropriately designed comparative clinical trials, for purposes of demonstrating bioequivalence. This approach is the least accurate, sensitive, and reproducible of the general approaches for measuring bioavailability or demonstrating bioequivalence. For dosage forms intended to deliver the active moiety to the bloodstream for systemic distribution, this approach may be considered acceptable only when analytical methods cannot be developed to permit use of one of the approaches outlined in paragraphs (1)(i) and (2) of this section, when the approaches described in paragraphs (1)(ii), (1)(iii), and (3) of this section are not available. This approach may also be considered sufficiently accurate for measuring bioavailability or demonstrating bioequivalence of dosage forms intended to deliver the active moiety locally, e.g., topical preparations for the skin, eye, and mucous membranes; oral dosage forms not intended to be absorbed, e.g., an antacid or radiopaque medium; and bronchodilators administered by inhalation if the onset and duration of pharmacological activity are defined. (5) A currently available in vitro test (for example a dissolution rate test) that ensures human in vivo bioavailability.

The term “biocompatible” as used herein refers to a material that is generally non-toxic to the recipient and does not possess any significant untoward effects to the subject and, further, that any metabolites or degradation products of the material are non-toxic to the subject. Typically a substance that is “biocompatible” causes no clinically relevant tissue irritation, injury, toxic reaction, or immunological reaction to living tissue.

The term “biodegradable” as used herein refers to a material that will erode to soluble species or that will degrade under physiologic conditions to smaller units or chemical species that are, themselves, non-toxic (biocompatible) to the subject and capable of being metabolized, eliminated, or excreted by the subject.

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components.

The term “chiral” is used to describe asymmetric molecules that are non-superposable since they are mirror images of each other and therefore have the property of chirality. Such molecules are also called enantiomers and are characterized by optical activity.

The term “chirality” refers to the geometric property of a rigid object (or spatial arrangement of points or atoms) of being non-superimposable on its mirror image; such an object has no symmetry elements of the second kind (a mirror plane, σ=S1, a center of inversion, i=S2, a rotation-reflection axis, S2n). If the object is superimposable on its mirror image, the object is described as being achiral.

The term “chirality axis” refers to an axis about which a set of ligands is held so that it results in a spatial arrangement which is not superimposable on its mirror image. For example, with an alkene abC≡C═Ccd, the chiral axis is defined by the C≡C═C bonds; and with an ortho-substituted biphenyl C-1, C-1′, C-4 and C-4′ lie on the chiral axis.

The term “chirality center” refers to an atom holding a set of ligands in a spatial arrangement, which is not superimposable on its mirror image. A chirality center may be considered a generalized extension of the concept of the asymmetric carbon atom to central atoms of any element.

The terms “chiroptic” or “chiroptical” refer to the optical techniques (using refraction, absorption or emission of anisotropic radiation) for investigating chiral substances (for example, measurements of optical rotation at a fixed wavelength, optical rotary dispersion (ORD), circular dichroism (CD) and circular polarization of luminescence (CPL)).

The term “chirotopic” refers to an atom (or point, group, face, etc. in a molecular model) that resides within a chiral environment. One that resides within an achiral environment has been called achirotopic.

The term “contact” and its various grammatical forms as used herein refers to a state or condition of touching or of immediate or local proximity.

The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations. Controlled release systems can deliver a drug substance at a predetermined rate for a definite time period. (Reviewed in Langer, R., “New methods of drug delivery,” Science, 249: 1527-1533 (1990); and Langer, R., “Drug delivery and targeting,” Nature, 392 (Supp.): 5-10 (1998)). Generally, release rates are determined by the design of the system, and are nearly independent of environmental conditions, such as pH. These systems also can deliver drugs for long time periods (days or years). Controlled release systems provide advantages over conventional drug therapies. For example, after ingestion or injection of standard dosage forms, the blood level of the drug rises, peaks and then declines. Since each drug has a therapeutic range above which it is toxic and below which it is ineffective, oscillating drug levels may cause alternating periods of ineffectiveness and toxicity. A controlled release preparation maintains the drug in the desired therapeutic range by a single administration. Other potential advantages of controlled release systems include: (i) localized delivery of the drug to a particular body compartment, thereby lowering the systemic drug level; (ii) preservation of medications that are rapidly destroyed by the body; (iii) reduced need for follow-up care; (iv) increased comfort; and (v) improved compliance. (Langer, R., “New methods of drug delivery,” Science, 249: at 1528).

Polymeric materials generally release drugs by the following mechanisms: (i) diffusion; (ii) chemical reaction, or (iii) solvent activation. The most common release mechanism is diffusion. In this approach, the drug is physically entrapped inside a solid polymer that can then be injected or implanted in the body. The drug then migrates from its initial position in the polymeric system to the polymer's outer surface and then to the body. There are two types of diffusion-controlled systems: reservoirs, in which a drug core is surrounded by a polymer film, which produce near-constant release rates, and matrices, where the drug is uniformly distributed through the polymer system. Drugs also can be released by chemical mechanisms, such as degradation of the polymer, or cleavage of the drug from a polymer backbone. Exposure to a solvent also can activate drug release; for example, the drug may be locked into place by polymer chains, and, upon exposure to environmental fluid, the outer polymer regions begin to swell, allowing the drug to move outward, or water may permeate a drug-polymer system as a result of osmotic pressure, causing pores to form and bringing about drug release. Such solvent-controlled systems have release rates independent of pH. Some polymer systems can be externally activated to release more drug when needed. Release rates from polymer systems can be controlled by the nature of the polymeric material (for example, crystallinity or pore structure for diffusion-controlled systems; the lability of the bonds or the hydrophobicity of the monomers for chemically controlled systems) and the design of the system (for example, thickness and shape). (Langer, R., “New methods of drug delivery,” Science, 249: at 1529).

Polyesters such as lactic acid-glycolic acid copolymers display bulk (homogeneous) erosion, resulting in significant degradation in the matrix interior. To maximize control over release, it is often desirable for a system to degrade only from its surface. For surface-eroding systems, the drug release rate is proportional to the polymer erosion rate, which eliminates the possibility of dose dumping, improving safety; release rates can be controlled by changes in system thickness and total drug content, facilitating device design. Achieving surface erosion requires that the degradation rate on the polymer matrix surface be much faster than the rate of water penetration into the matrix bulk. Theoretically, the polymer should be hydrophobic but should have water-labile linkages connecting monomers. For example, it was proposed that, because of the lability of anhydride linkages, polyanhydrides would be a promising class of polymers. By varying the monomer ratios in polyanhydride copolymers, surface-eroding polymers lasting from 1 week to several years were designed, synthesized and used to deliver nitrosoureas locally to the brain. ((Langer, R., “New methods of drug delivery,” Science, 249: at 1531 citing, Rosen et al, Biomaterials 4, 131 (1983); Leong et al, J. Biomed. Mater. Res. 19, 941 (1985); Domb et al, Macromolecules 22, 3200 (1989); Leong et al, J. Biomed. Mater. Res. 20, 51 (1986), Brem et al, Selective Cancer Ther. 5, 55 (1989); Tamargo et al, J. Biomed. Mater. Res. 23, 253 (1989)).

Several different surface-eroding polyorthoester systems have been synthesized. Additives are placed inside the polymer matrix, which causes the surface to degrade at a different rate than the rest of the matrix. Such a degradation pattern can occur because these polymers erode at very different rates, depending on pH, and the additives maintain the matrix bulk at a pH different from that of the surface. By varying the type and amount of additive, release rates can be controlled. ((Langer, R., “New methods of drug delivery,” Science, 249: at 1531 citing. Heller, et al, in Biodegradable Polymers as Drug Delivery Systems, M. Chasin and R. Langer, Eds (Dekker, New York, 1990), pp. 121-161)). Polymeric materials used in controlled release drug delivery systems include poly (α-hydroxyacids), acrylic, polyanhydrides and other polymers, such as polycaprolactone, ethylcellulose, polystyrene, etc.

The term “cosmetic composition” as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The term “cosmetically acceptable carrier” as used herein refers to a substantially non-toxic carrier, conventionally useable for the topical administration of cosmetics, with which compounds will remain stable and bioavailable.

The term “covalently linked” as used herein refers to a form of chemical bonding characterized by the sharing of electrons between atoms whereby the attractive and repulsive forces between the atoms is stably balanced.

The term “cream” as used herein refers to a viscous liquid or semisolid emulsion of either the oil-in-water or water-in-oil type. As used herein, “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent. Common types of emulsions are oil-in-water, where oil is the dispersed liquid and an aqueous solution, such as water, is the dispersion medium, and water-in-oil, where, conversely, an aqueous solution is the dispersed phase. It also is possible to prepare emulsions that are nonaqueous. Creams of the oil-in-water type include hand creams and foundation creams. Water-in-oil creams include cold creams and emollient creams. The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds. Common emollients used in the manufacture of cosmetics include, but are not limited to, butters, such as Aloe Butter, Almond Butter, Avocado Butter, Cocoa Butter, Coffee Butter, Hemp Seed Butter, Kokum Butter, Mango Butter, Mowrah Butter, Olive Butter, Sal Butter, Shea Butter, glycerin, and oils, such as Almond Oil, Aloe Vera Oil, Apricot Kernel Oil, Avocado Oil, Babassu Oil, Black Cumin Seed Oil, Borage Seed Oil, Brazil Nut Oil, Camellia Oil, Castor Oil, Coconut Oil, Emu Oil, Evening Primrose Seed Oil, Flaxseed Oil, Grape Seed Oil, Hazelnut Oil, Hemp Seed Oil, Jojoba Oil, Kukui Nut Oil, Macadamia Nut Oil, Meadowfoam Seed Oil, Mineral Oil, Neem Seed Oil, Olive Oil, Palm Oil, Palm Kernel Oil, Peach Kernel Oil, Peanut Oil, Plum Kernel Oil, Pomegranate Seed Oil, Poppy Seed Oil, Pumpkin Seed Oil, Rice Bran Oil, Rosehip Seed Oil, Safflower Oil, Sea Buckthorn Oil, Sesame Seed Oil, Shea Nut Oil, Soybean Oil, Sunflower Oil, Tamanu Oil, Turkey Red Oil, Walnut Oil, Wheatgerm Oil.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

The term “dendrimer” as used herein refers to a nano-sized, radially symmetric molecule with well-defined homogeneous and monodisperse structures consisting of tree-like arms or branches. Dendromers contain symmetric branching units built around a small molecule or a linear polymer core. The dendrimer grows outward from a multifunctional core molecule, which reacts with monomer molecules containing one reactive and two dormant groups. The new periphery of the molecule can be activated for reactions with more monomers.

The term “derivative” as used herein means a compound that may be produced from another compound of similar structure in one or more steps. A “derivative” or “derivatives” of a compound retains at least a degree of the desired function of the compound. Accordingly, an alternate term for “derivative” may be “functional derivative.” Derivatives can include chemical modifications of the compound, such as akylation, acylation, carbamylation, iodination or any modification that derivatizes the compound. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine.

“Differential scanning calorimetry (DSC)” is a thermoanalytical technique useful in detecting phase transitions in solid samples by measuring the amount of heat absorbed or released during such transitions.

Dose-effect curves. The intensity of effect of a drug (y-axis) can be plotted as a function of the dose of drug administered (X-axis). (Goodman & Gilman's The Pharmacological Basis of Therapeutics, Ed. Joel G. Hardman, Lee E. Limbird, Eds., 10th Ed., McGraw Hill, New York (2001), p. 25, 50). These plots are referred to as dose-effect curves. Such a curve can be resolved into simpler curves for each of its components. These concentration-effect relationships can be viewed as having four characteristic variables: potency, slope, maximal efficacy, and individual variation.

The location of the dose-effect curve along the concentration axis is an expression of the potency of a drug. Id. For example, if the drug is to be administered by transdermal absorption, a highly potent drug is required, since the capacity of the skin to absorb drugs is limited.

The slope of the dose-effect curve reflects the mechanism of action of a drug. The steepness of the curve dictates the range of doses useful for achieving a clinical effect.

The term “maximal or clinical efficacy” refers to the maximal effect that can be produced by a drug. Maximal efficacy is determined principally by the properties of the drug and its receptor-effector system and is reflected in the plateau of the curve. In clinical use, a drug's dosage may be limited by undesired effects.

Biological variability. An effect of varying intensity may occur in different individuals at a specified concentration or a drug. It follows that a range of concentrations may be required to produce an effect of specified intensity in all subjects.

Lastly, different individuals may vary in the magnitude of their response to the same concentration of a drug when the appropriate correction has been made for differences in potency, maximal efficacy and slope.

The duration of a drug's action is determined by the time period over which concentrations exceed the minimum effective concentration (MEC). Following administration of a dose of drug, its effects usually show a characteristic temporal pattern. A plot of drug effect vs. time illustrates the temporal characteristics of drug effect and its relationship to the therapeutic window. A lag period is present before the drug concentration exceeds the MEC for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. The therapeutic window reflects a concentration range that provides efficacy without unacceptable toxicity. Generally another dose of drug can be administered to maintain concentrations within the therapeutic window over time. The terms “formulation” and “composition” are used interchangeably herein to refer to a product of the described invention that comprises all active and inert ingredients.

The term “full-thickness skin” as used herein refers to skin containing both the epidermis and the entire thickness of the dermis.

The term “gel” as used herein refers to a sticky, jelly-like semisolid or solid prepared from high molecular weight polymers in an aqueous or alcoholic base. Alcoholic gels are drying and cooling, while non-alcoholic gels are more lubricating and are well suited, for example, to dry scaling lesions. Due to their drying effect, especially from those gels containing alcohol, gels may cause irritation and cracking of the skin. Starches and aloe are commonly used agents in the manufacture of gelled cosmetic preparations.

The term “hydrophilic” as used herein refers to a material or substance having an affinity for polar substances, such as water.

The term “hydrophobic” as used herein refers to a material or substance having an affinity for nonpolar or neutral substances.

The term “inclusion complex” as used herein refers to an entity consisting of two or more molecules in which a host molecule contains a guest molecule, either totally or in part, using only physical forces. No covalent bonding is involved. Cyclodextrins are typical host molecules and can contain a variety of guest molecules and compounds. The inserted compound of the inclusion complex is considered “complexed” with the cyclodextrin. A compound that is not part of an inclusion complex is considered “alone” or “non-complexed.”

The term “irritant” as used herein refers to a material that acts locally on the skin to induce, based on irritant concentration, hyperemia (meaning an excess of blood in an area or body part, usually indicated by red, flushed color or heat in the area), inflammation, and desiccation. Irritant agents include, but are not limited to, alcohol, aromatic ammonia spirits, benzoin tincture, camphor capsicum, and coal tar extracts.

The term “isolated” is used herein to refer to a material, such as, but not limited to, a compound, nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “isomer” as used herein refers to one of two or more molecules having the same number and kind of atoms and hence the same molecular weight, but differing in chemical structure. Isomers may differ in the connectivities of the atoms (structural isomers), or they may have the same atomic connectivities but differ only in the arrangement or configuration of the atoms in space (stereoisomers). Stereoisomers may include, but are not limited to, E/Z double bond isomers, enantiomers, and diastereomers. Structural moieties that, when appropriately substituted, can impart stereoisomerism include, but are not limited to, olefinic, imine or oxime double bonds; tetrahedral carbon, sulfur, nitrogen or phosporus atoms; and allenic groups. Enantiomers are non-superimposable mirror images. A mixture of equal parts of the optical forms of a compound is known as a racemic mixture or racemate. Diastereomers are stereoisomers that are not mirror images. The invention provides for each pure stereoisomer of any of the compounds described herein. Such stereoisomers may include enantiomers, diasteriomers, or E or Z alkene, imine or oxime isomers. The invention also provides for stereoisomeric mixtures, including racemic mixtures, diastereomeric mixtures, or E/Z isomeric mixtures. Stereoisomers can be synthesized in pure form (Nógrádi, M.; Stereoselective Synthesis, (1987) VCH Editor Ebel, H. and Asymmetric Synthesis, Volumes 3-5, (1983) Academic Press, Editor Morrison, J.) or they can be resolved by a variety of methods such as crystallization and chromatographic techniques (Jaques, J.; Collet, A.; Wilen, S.; Enantiomer, Racemates, and Resolutions, 1981, John Wiley and Sons and Asymmetric Synthesis, Vol. 2, 1983, Academic Press, Editor Morrison, J). In addition the compounds of the described invention may be present as enantiomers, diasteriomers, isomers or two or more of the compounds may be present to form a racemic or diastereomeric mixture.

The phrase “localized administration”, as used herein, refers to administration of a therapeutic agent in a particular location in the body that may result in a localized pharmacologic effect. Local delivery of a bioactive agent to locations such as organs, cells or tissues can also result in a therapeutically useful, long-lasting presence of a bioactive agent in those local sites or tissues, since the routes by which a bioactive agent is distributed, metabolized, and eliminated from these locations may be different from the routes that define the pharmacokinetic duration of a bioactive agent delivered to the general systemic circulation.

The term “localized pharmacologic effect”, as used herein, refers to a pharmacologic effect limited to a certain location, i.e. in proximity to a certain location, place, area or site. The phrase “predominantly localized pharmacologic effect”, as used herein, refers to a pharmacologic effect of a drug limited to a certain location by at least 1 to 3 orders of magnitude achieved with a localized administration as compared to a systemic administration.

The term “long-term” release, as used herein, refers to an implant constructed and arranged to deliver therapeutic levels of the active ingredient for at least 7 days, and preferably about 30 to about 60 days.

The terms “minimum effective concentration”, “minimum effective dose”, or “MEC” are used interchangeably to refer to the minimum concentration of a drug required to produce a desired pharmacological effect in most patients.

The term “maximum tolerated dose” as used herein refers to the highest dose of a drug that does not produce unacceptable toxicity.

The term “optical rotation” refers to the change of direction of the plane of polarized light to either the right or the left as it passes through a molecule containing one or more asymmetric carbon atoms or chirality centers. The direction of rotation, if to the right, is indicated by either a plus sign (+) or a d-; if to the left, by a minus (−) or an l-. Molecules having a right-handed configuration (D) usually are dextrorotatory, D(+), but may be levorotatory, L(−). Molecules having left-handed configuration (L) are usually levorotatory, L(−), but may be dextrorotatory, D(+). Compounds with this property are said to be optically active and are termed optical isomers. The amount of rotation of the plane of polarized light varies with the molecule but is the same for any two isomers, though in opposite directions.

The term “parenteral” as used herein refers to a route of administration where the drug or agent enters the body without going through the stomach or “gut”, and thus does not encounter the first pass effect of the liver. Examples include, without limitation, introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecal\hat{ } (i.e., an injection into the space around the spinal cord or under the arachnoid membrane of the brain), intraventricular injection, intracisternal injection, or infusion techniques. A parenterally administered composition is delivered using a needle.

The term “particles” as used herein refers to an extremely small constituent that may contain in whole or in part at least one active agent complexed with HPBCD as described herein. The term “microparticle” is used herein to refer generally to a variety of substantially spherical structures having sizes from about 10 nm to 2000 microns (2 millimeters) and includes microcapsule, microparticle, nanoparticle, nanocapsule, nanosphere as well as particles, in general, that are less than about 2000 microns (2 millimeters). The particles may contain the inclusion complexes in a core surrounded by a coating. The inclusion complexes also may be dispersed throughout the particles or adsorbed onto the particles. The particles may be of any order release kinetics, including zero order release, first order release, second order release, delayed release, sustained release, immediate release, etc., and any combination thereof. The particles may further include any of those materials routinely used in the art of pharmacy and medicine, including, but not limited to, erodible, nonerodible, biodegradable, or nonbiodegradable material or combinations thereof. The particles may be microcapsules that contain the inclusion complexes in solution or in a semisolid state. The particles may be of virtually any shape.

The term “penetration” and its various grammatical forms as used herein refers to delivery of a substance through the skin.

The term “penetration enhancer” as used herein refers to an agent known to accelerate the delivery of a substance through the skin.

“Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cᵥₑₕ×Kₘ·D/x [Formula 1]

where J=rate of absorption

Cᵥₑₕ=concentration of drug in vehicle

Kₘ=partition coefficient

D=diffusion coefficient.

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Increased skin temperature increases permeability; and (vi) The composition of the compound and of the vehicle also determines the absorbency of a substance. Most substances applied topically are incorporated into bases or vehicles. The vehicle chosen for a topical application will greatly influence absorption, and may itself have a beneficial effect on the skin. Factors that determine the choice of vehicle and the transfer rate across the skin are the substance's partition coefficient, molecular weight and water solubility. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460.

The term “pharmaceutical composition” is used herein to refer to a composition that is employed to prevent, reduce in intensity, cure or otherwise treat a target condition or disease.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated. The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth metal salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer.

The term “process” as used herein refers to a series of operations, actions and controls used to manufacture a drug product.

The term “pulsatile release” as used herein refers to any drug-containing formulation in which a burst of the drug is released at one or more predetermined time intervals.

The term “purification” and its various grammatical forms as used herein refers to the process of isolating or freeing from foreign, extraneous, or objectionable elements.

The term “racemate” as used herein refers to an equimolar mixture of two optically active components that neutralize the optical effect of each other and is therefore optically inactive.

The term “release” and its various grammatical forms, refers to dissolution of an active drug component and diffusion of the dissolved or solubilized species by a combination of the following processes: (1) hydration of the cyclodextrin, (2) diffusion of a solution into the cyclodextrin; (3) dissolution of the drug; and (4) diffusion of the dissolved drug out of the cyclodextrin.

The term “retention rate” or “RR” as used herein refers to the proportion of patients who maintain the same drug in a given time period. Drug retention rate is a tool for evaluating the effectiveness and safety of treatments.

The term “same” as used herein refers to agreeing in kind, amount; unchanged in character or condition.

The term “similar” as used herein refers to having a general likeness.

The term “skin” as used herein refers to the largest organ in the body consisting of several layers. which plays an important role in biologic homeostasis, and is comprised of the epidermis and the dermis. The epidermis, which is composed of several layers beginning with the stratum corneum, is the outermost layer of the skin, and the innermost skin layer is the deep dermis. The skin has multiple functions, including thermal regulation, metabolic function (vitamin D metabolism), and immune functions. FIG. 1 presents a diagram of skin anatomy.

In humans, the usual thickness of the skin is from 1-2 mm, although there is considerable variation in different parts of the body. The relative proportions of the epidermis and dermis also vary, and a thick skin is found in regions where there is a thickening of either or both layers. For example, on the interscapular (between the shoulder blades) region of the back, where the dermis is particularly thick, the skin may be more than 5 mm thick, whereas on the eyelids it may be less than 0.5 mm. Generally, the skin is thicker on the dorsal or extensor surfaces of the body than on the ventral or flexor surfaces; however, this is not the case for the hands and feet. The skin of the palms and soles is thicker than on any dorsal surface except the intrascapular region. The palms and soles have a characteristically thickened epidermis, in addition to a thick dermis

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy. On the thick skin of the palms and soles, the fields form long, narrow ridges separated by parallel coursing furrows, and in the fingertips these ridges are arranged in the complicated loops, whorls (verticil) and spirals that give the fingerprints characteristic for each individual. These ridges are more prominent in those regions where the epidermis is thickest.

Where there is an epidermal ridge externally there is a corresponding narrower projection, called a “rete peg,” on the dermal surface. Dermal papillae on either side of each rete peg project irregularly into the epidermis. In the palms and soles, and other sensitive parts of the skin, the dermal papillae are numerous, tall and often branched, and vary in height (from 0.05 mm to 0.2 mm). Where mechanical demands are slight and the epidermis is thinner, such as on the abdomen and face, the papillae are low and fewer in number.

The epidermis provides the body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically. The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

The stratum corneum is an avascular, multilayer structure that functions as a barrier to the environment and prevents transepidermal water loss. Recent studies have shown that enzymatic activity is involved in the formation of an acid mantle in the stratum corneum. Together, the acid mantle and stratum corneum make the skin less permeable to water and other polar compounds, and indirectly protect the skin from invasion by microorganisms. Normal surface skin pH is between 4 and 6.5 in healthy people; it varies according to area of skin on the body. This low pH forms an acid mantle that enhances the skin barrier function.

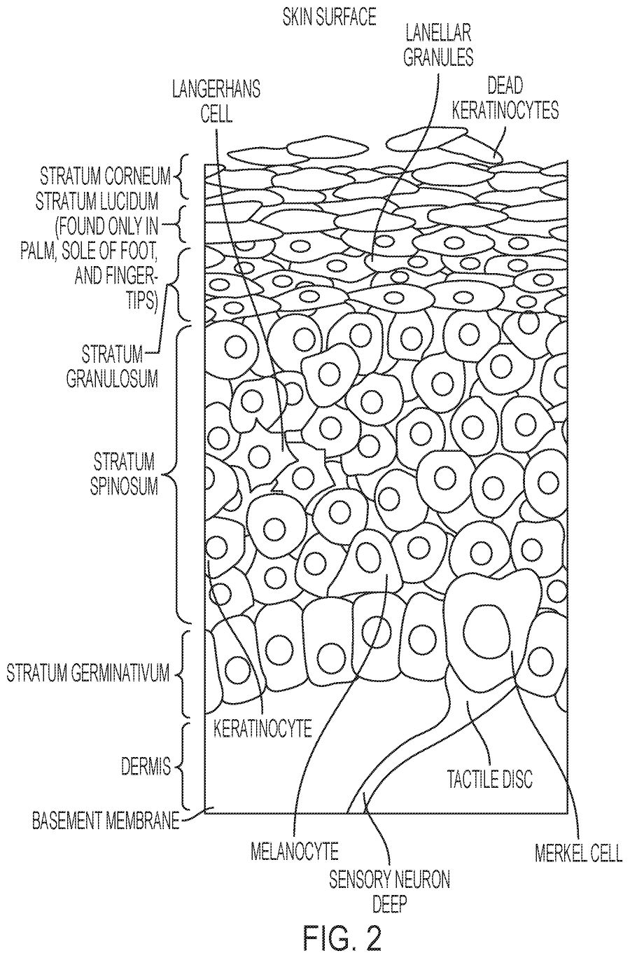

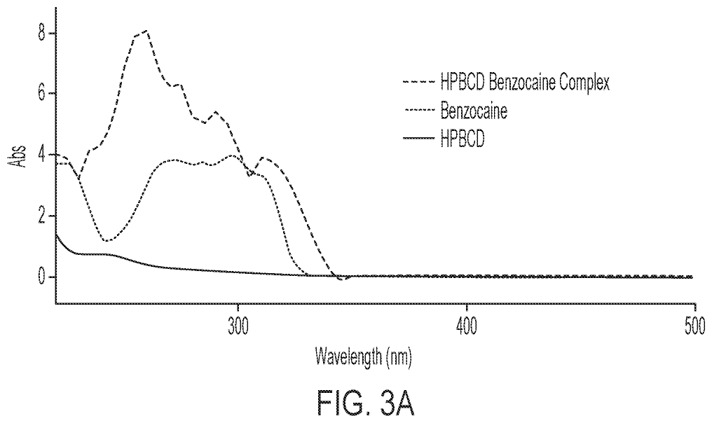

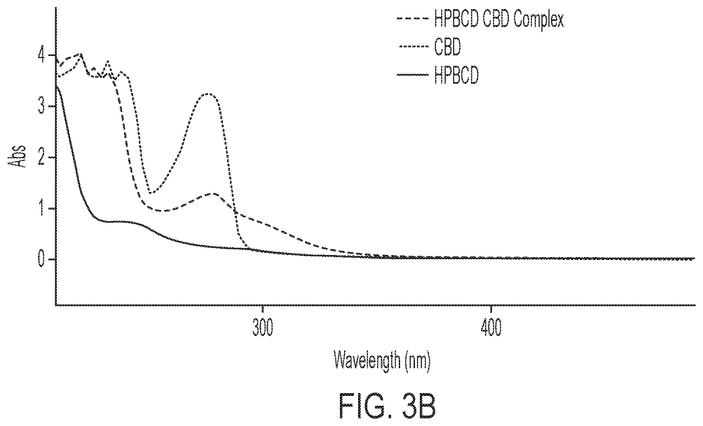

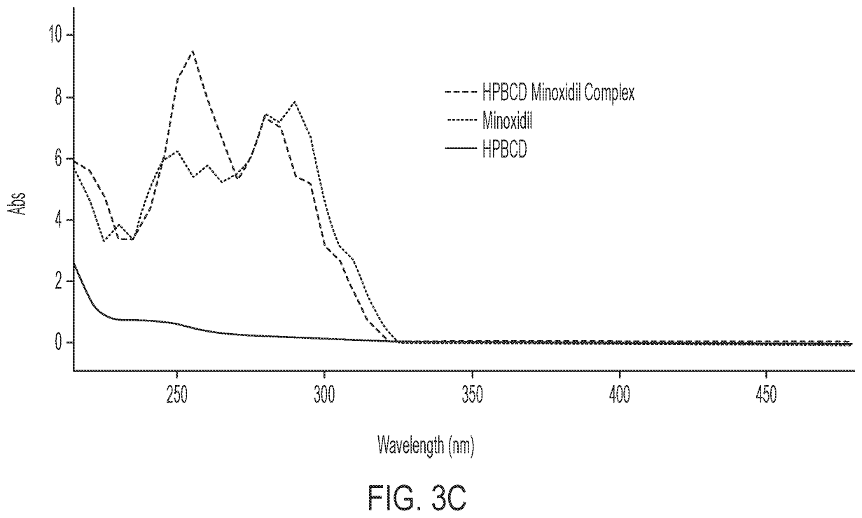

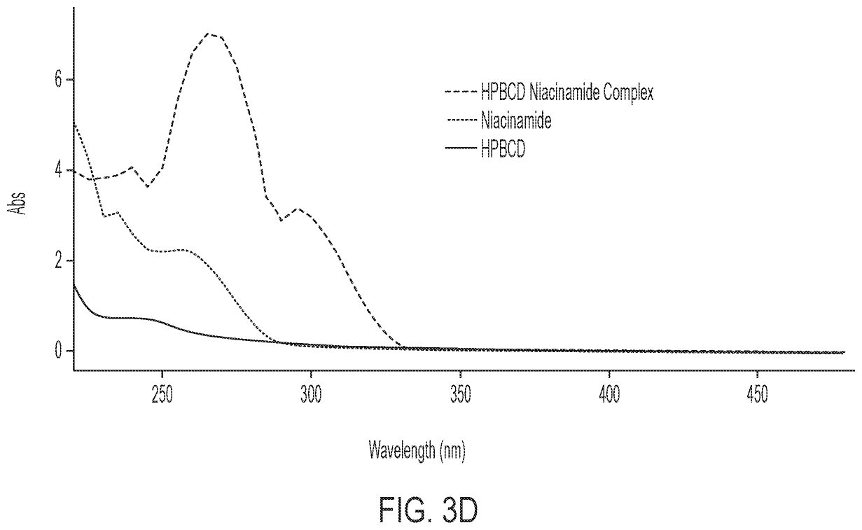

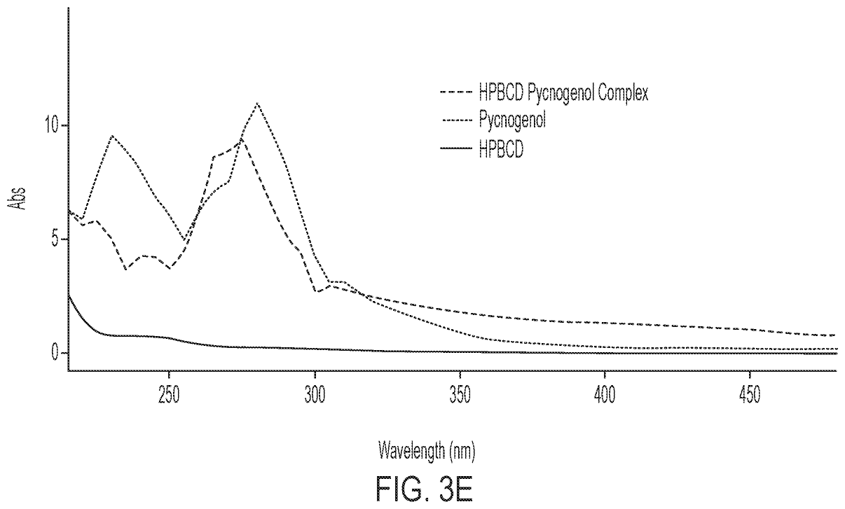

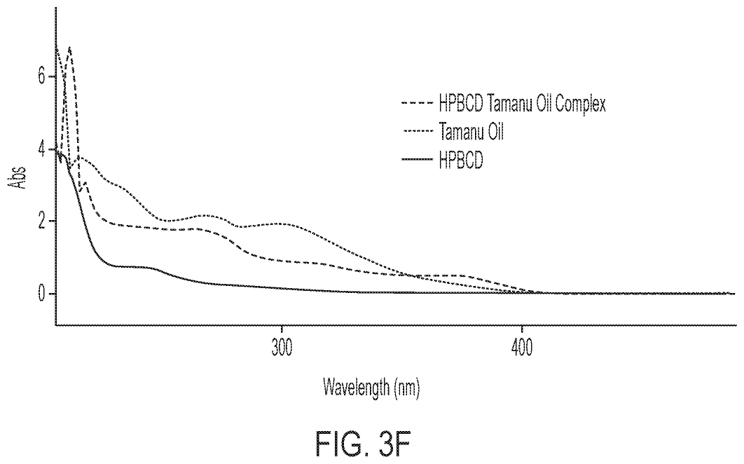

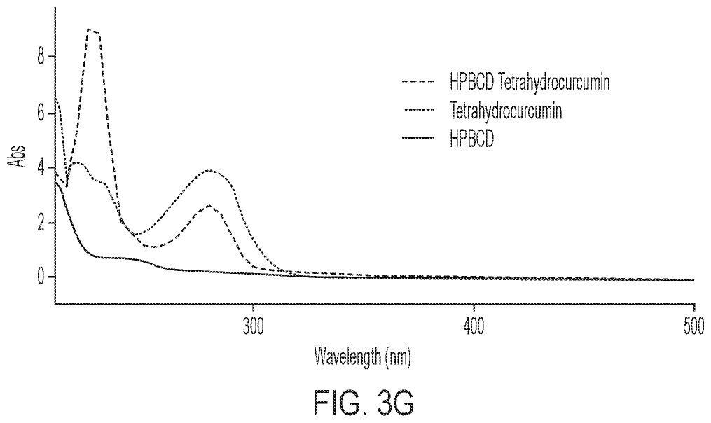

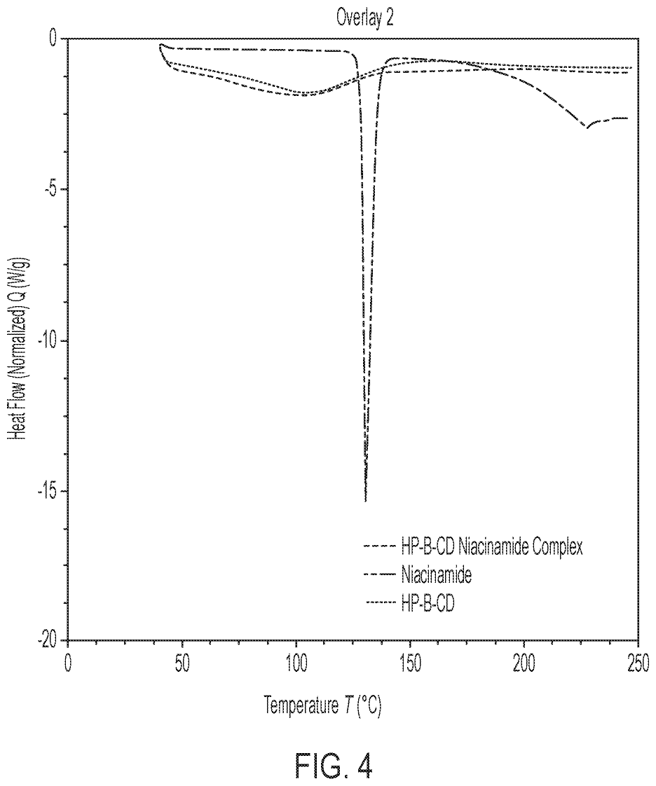

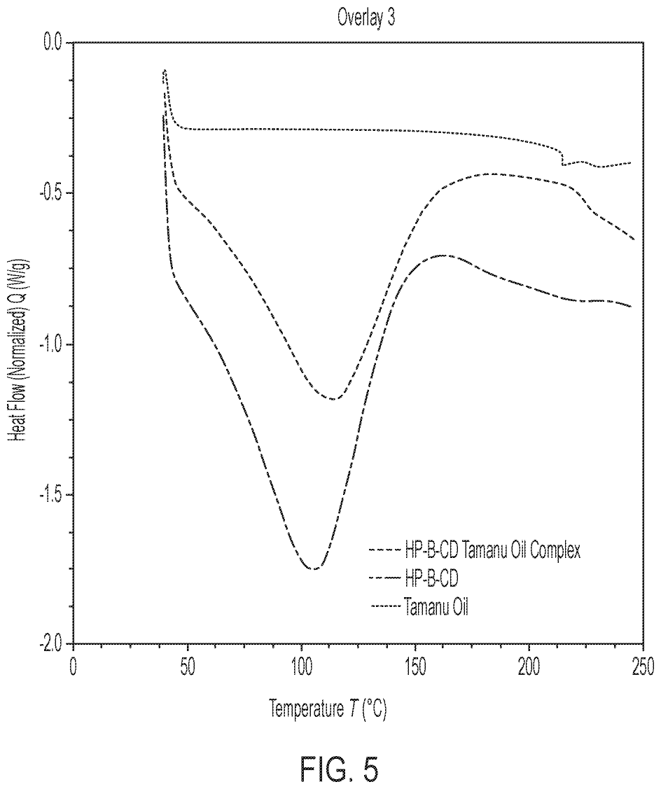

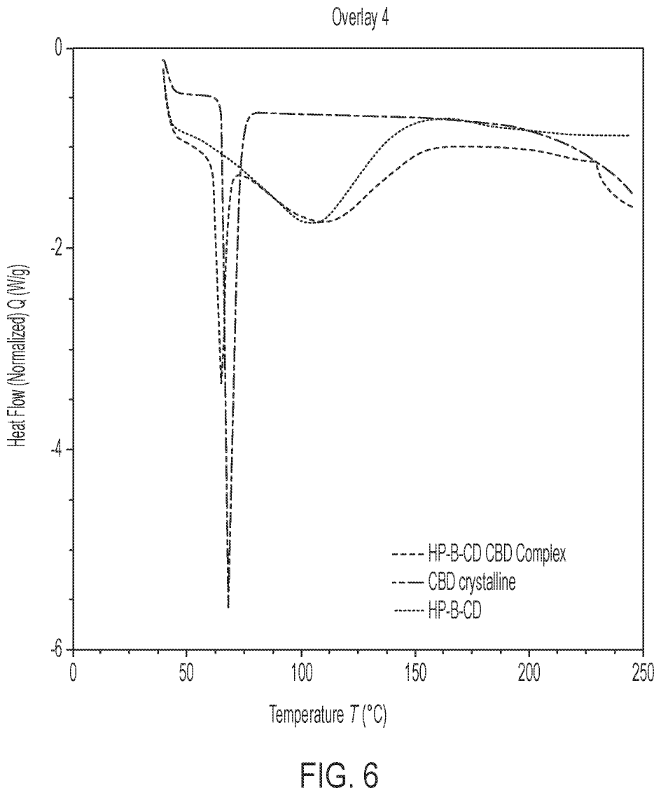

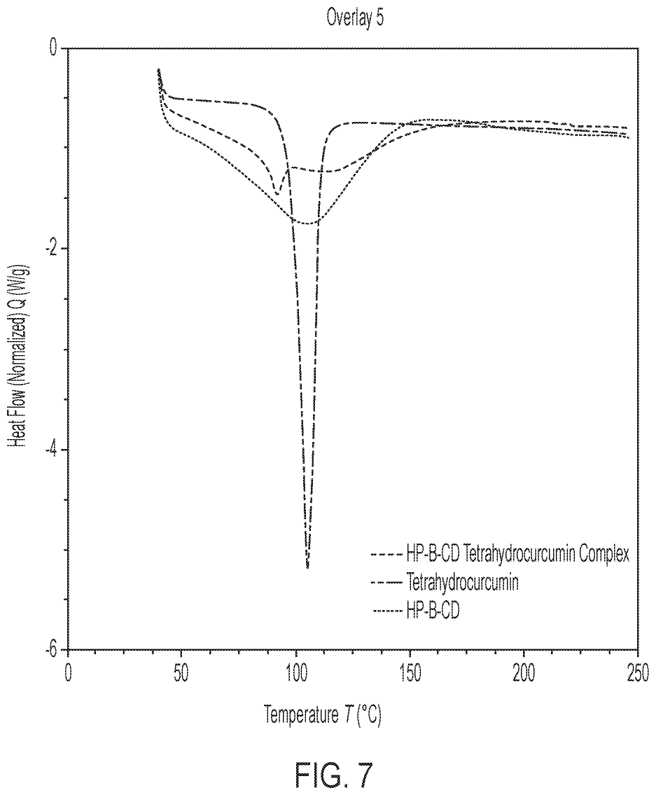

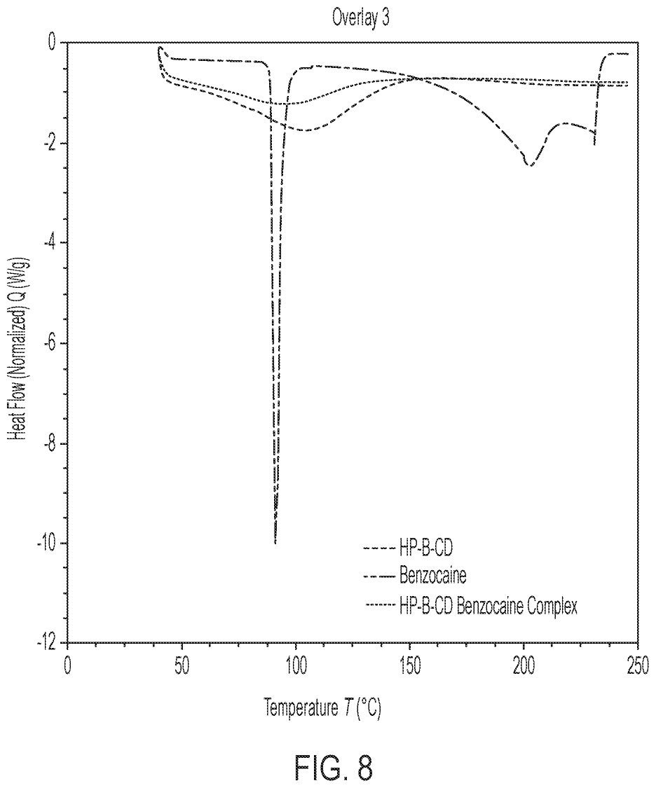

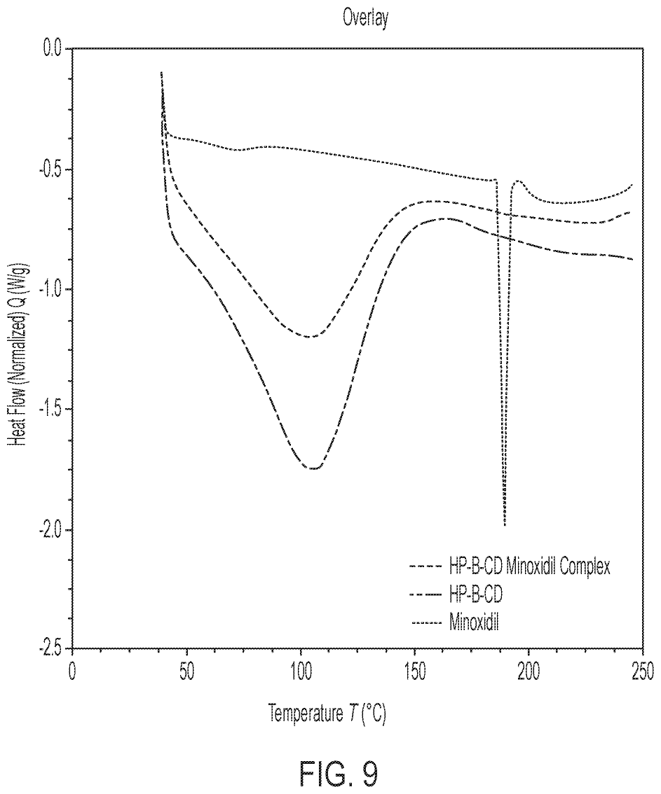

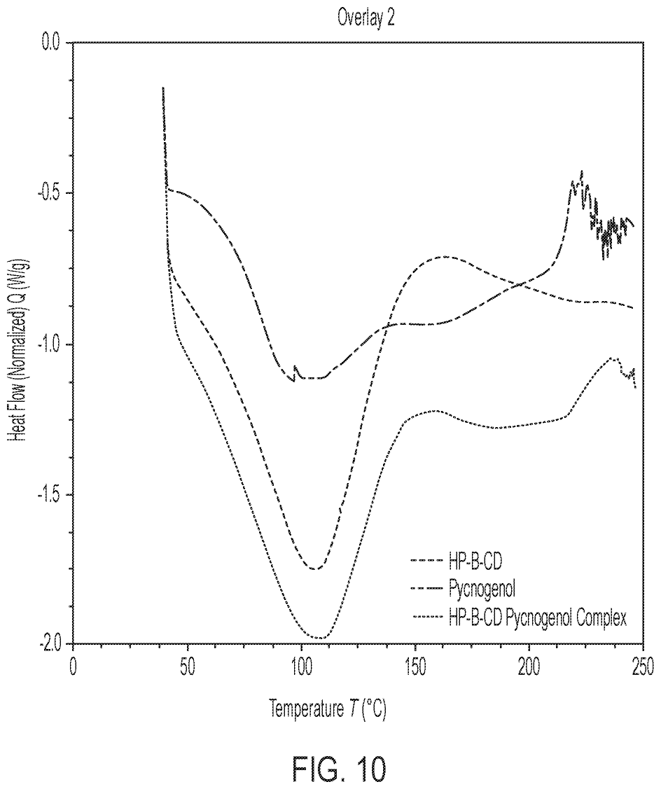

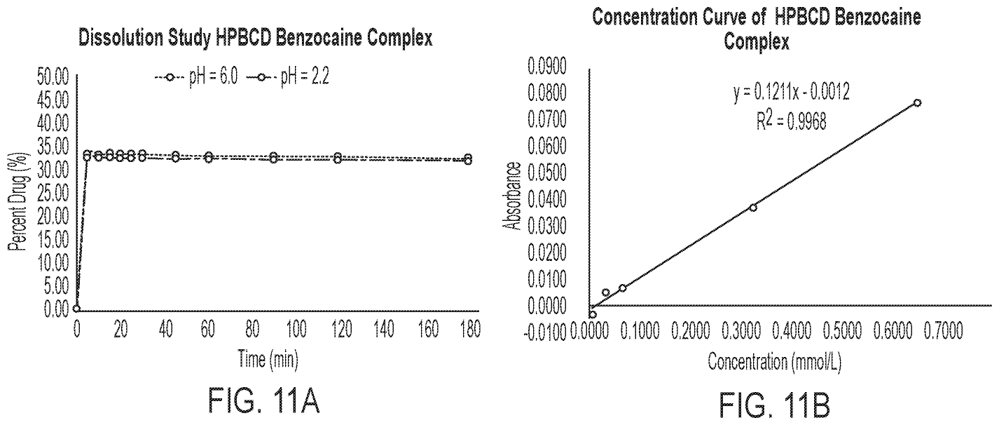

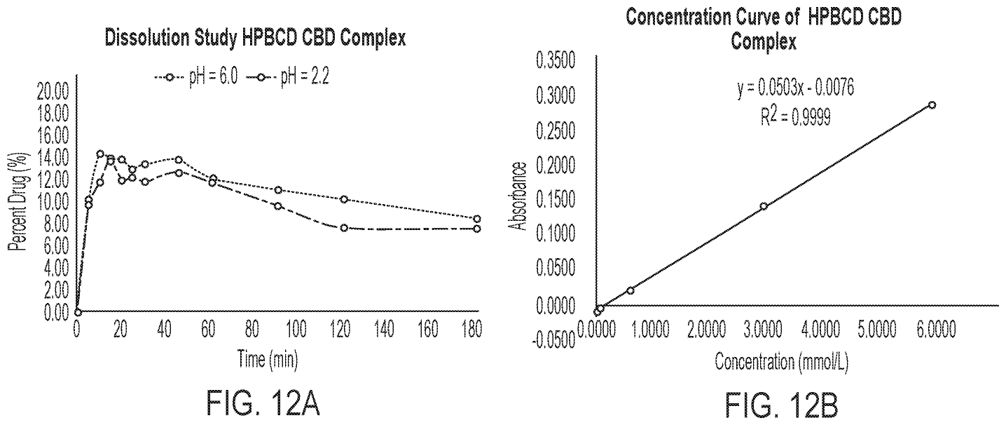

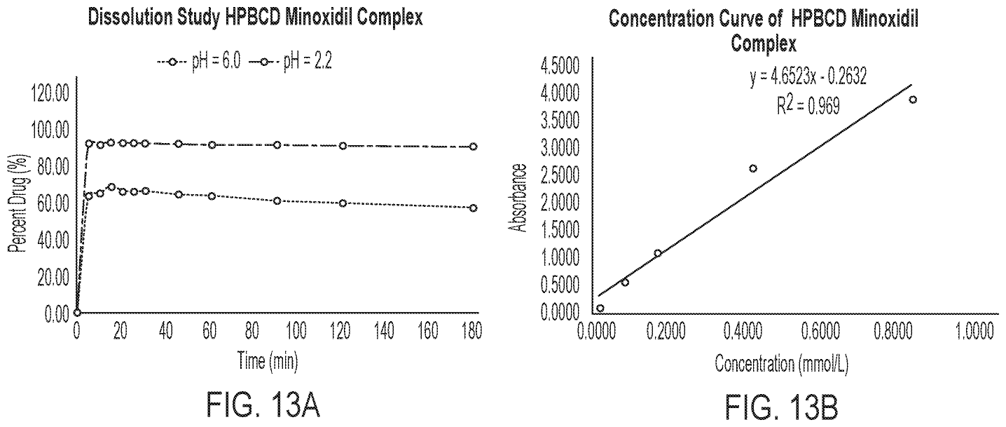

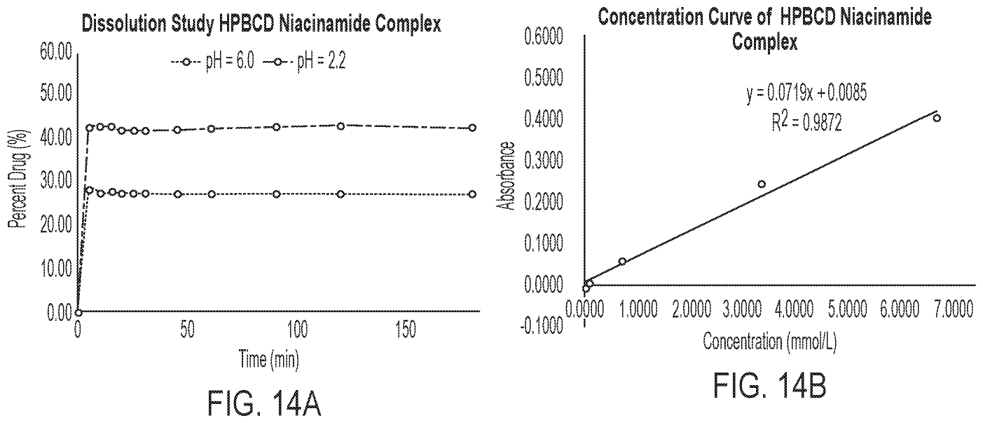

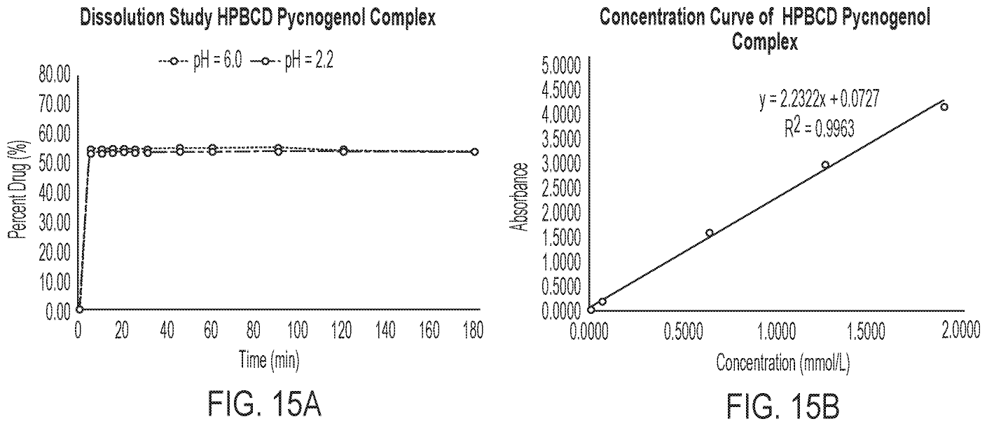

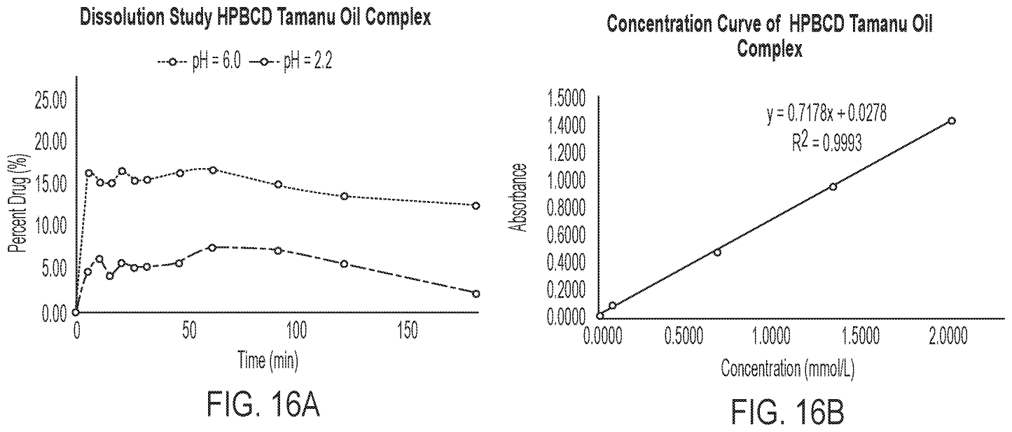

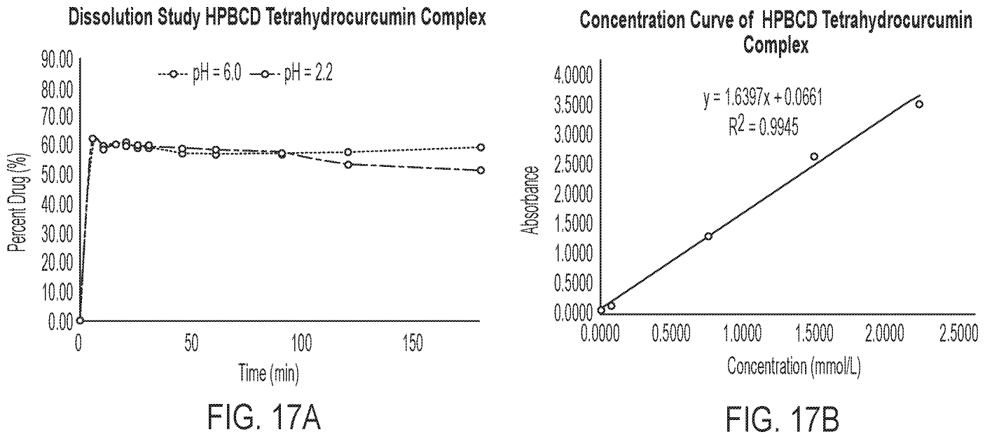

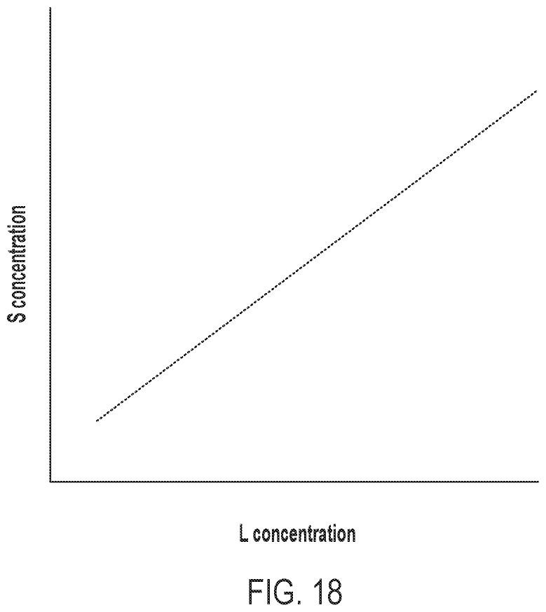

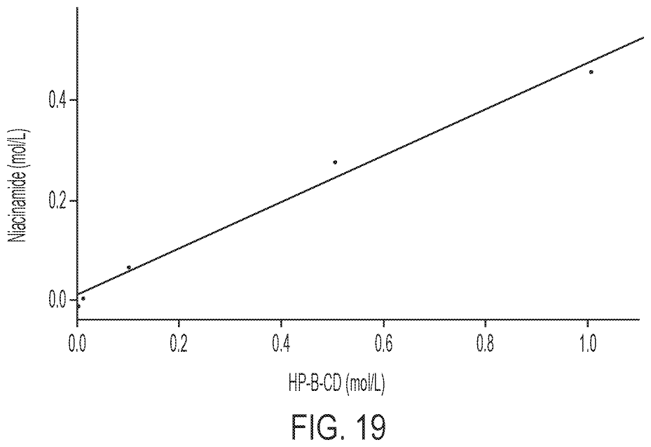

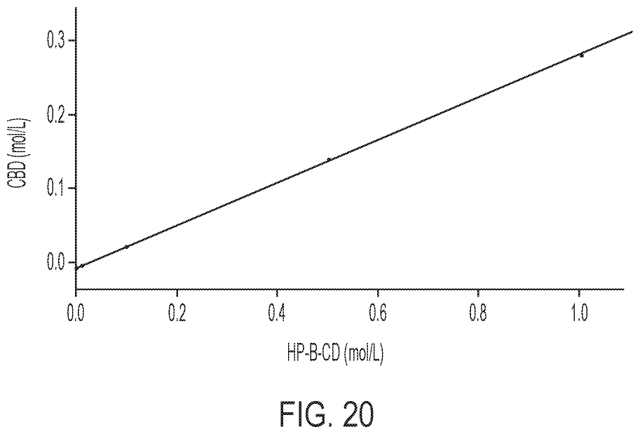

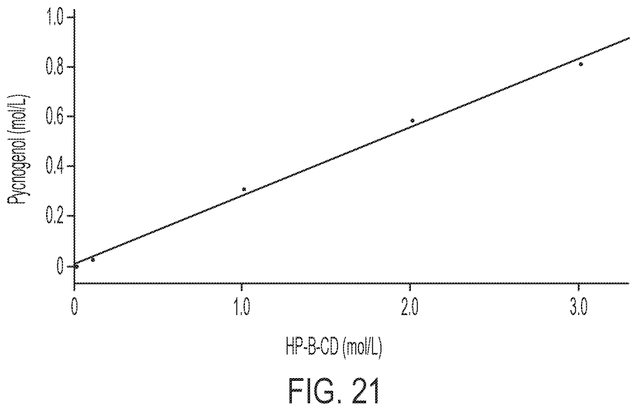

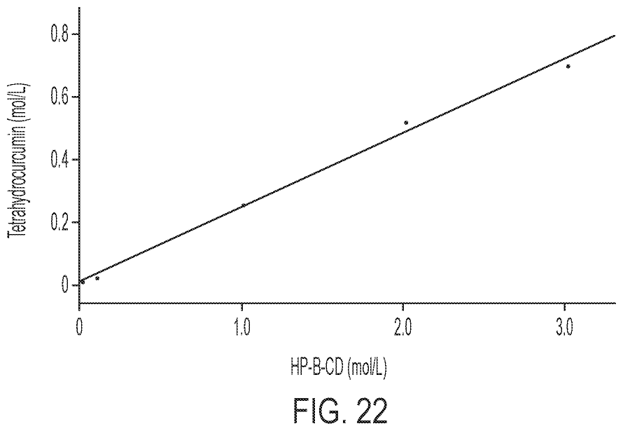

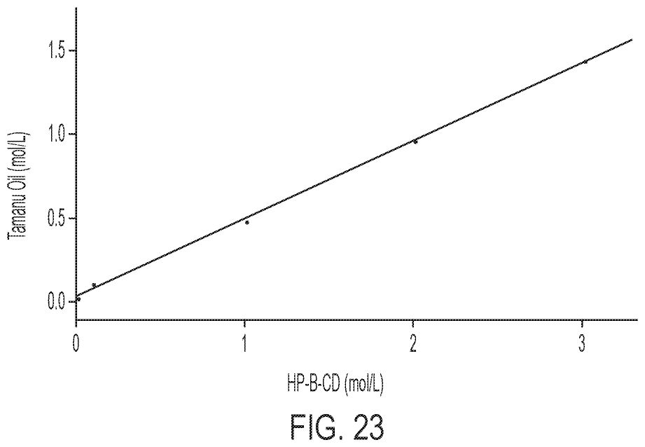

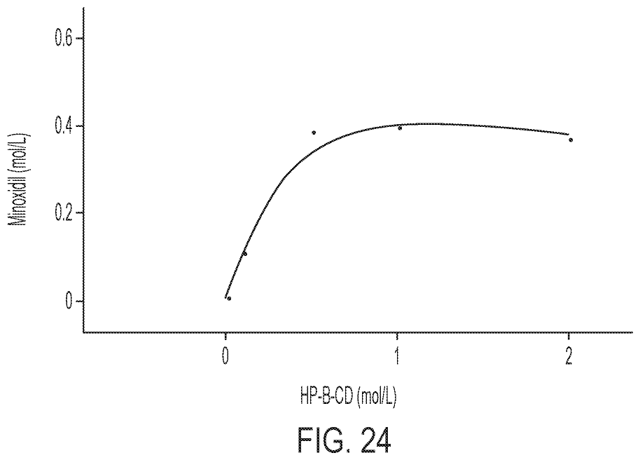

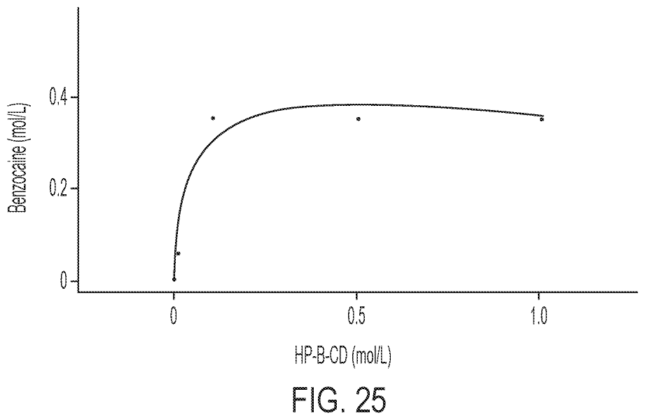

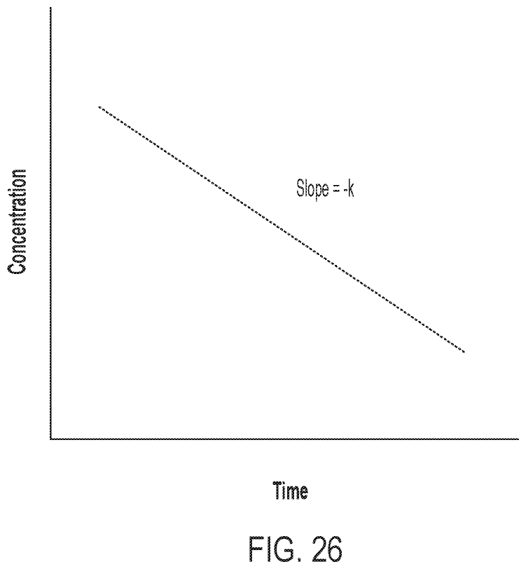

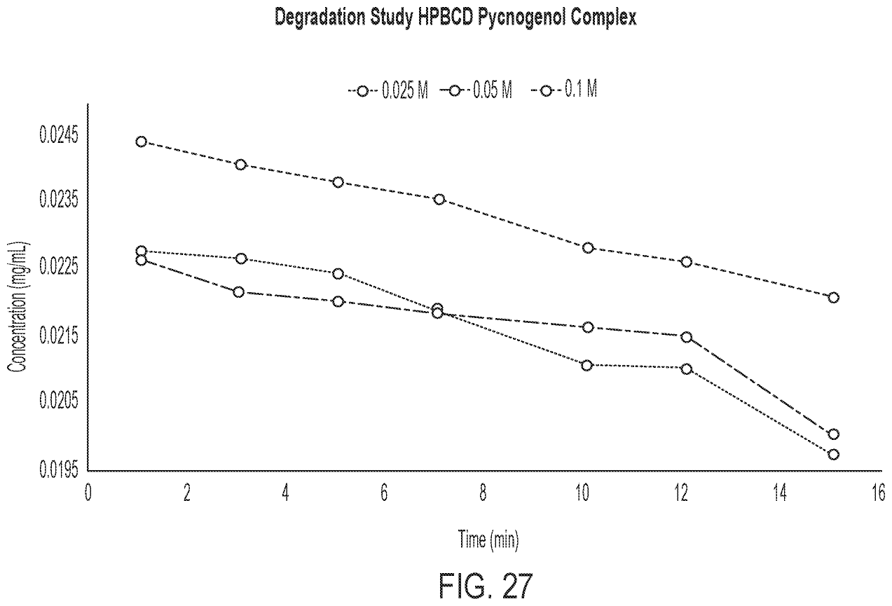

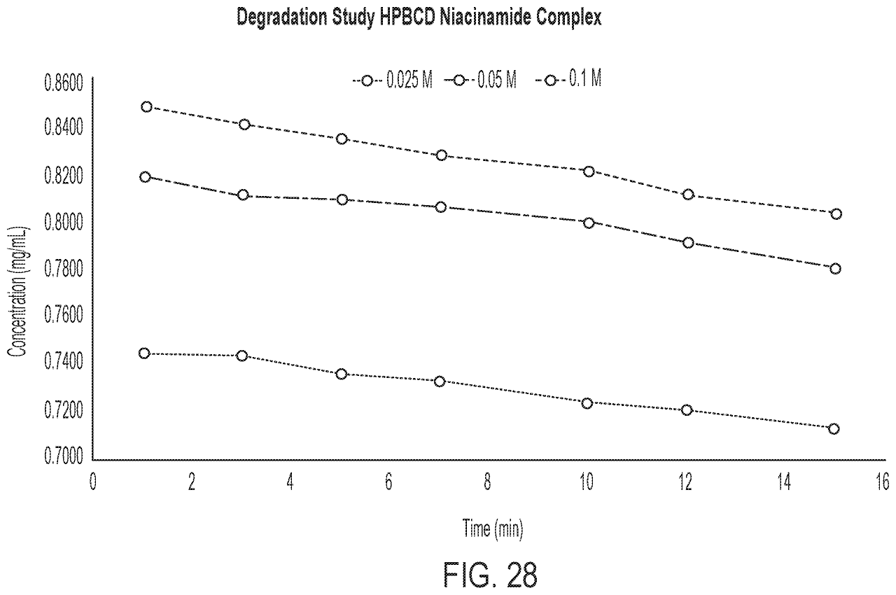

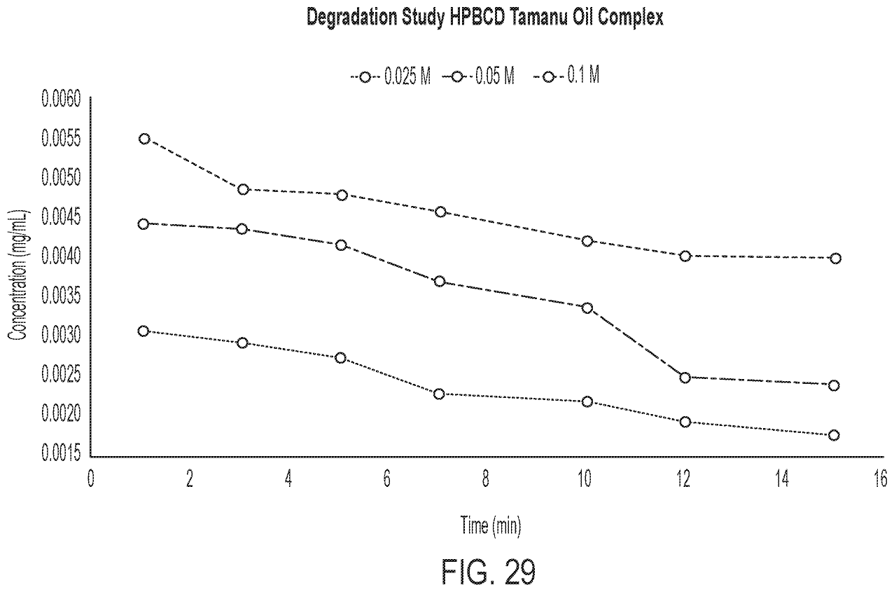

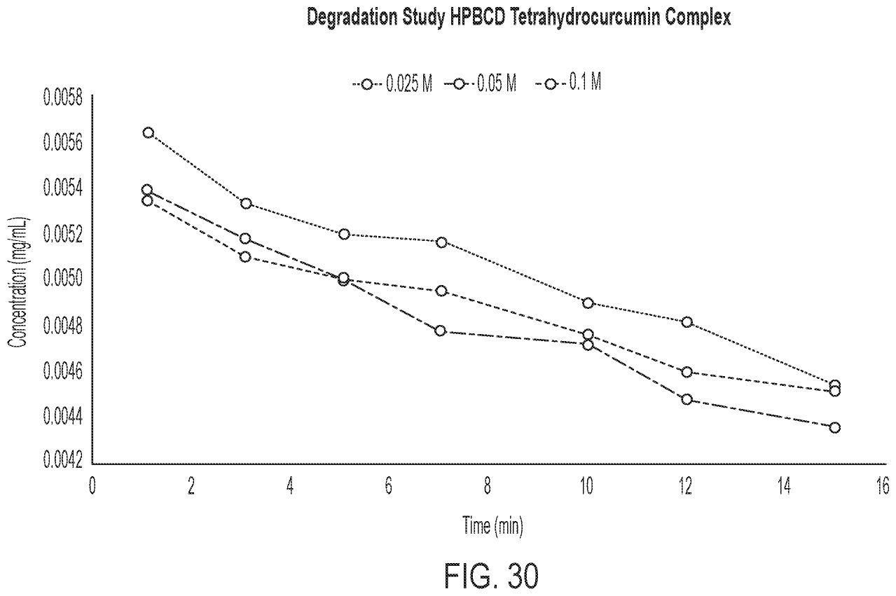

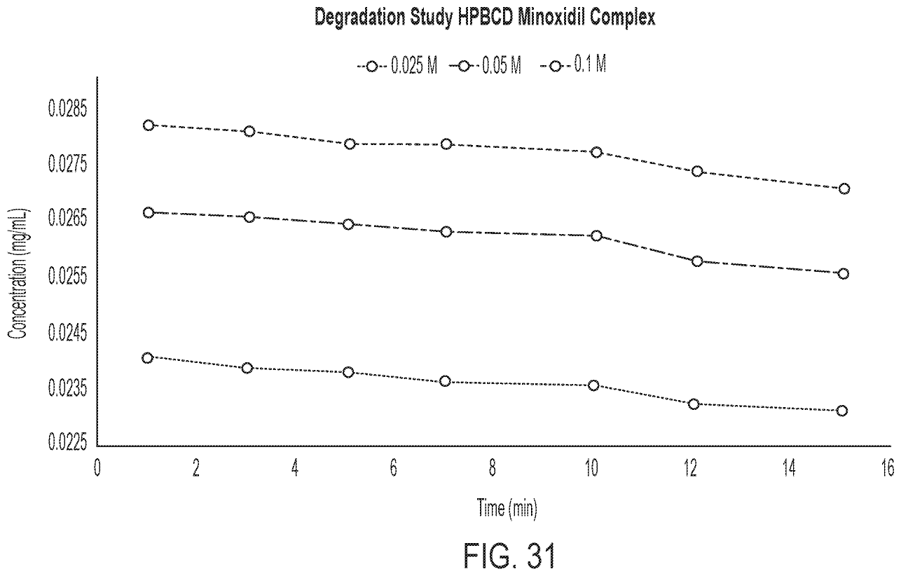

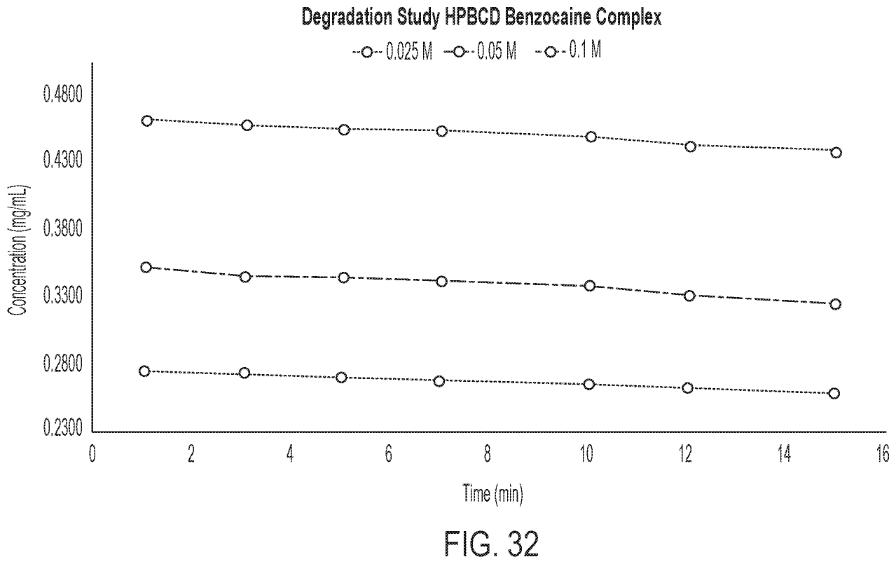

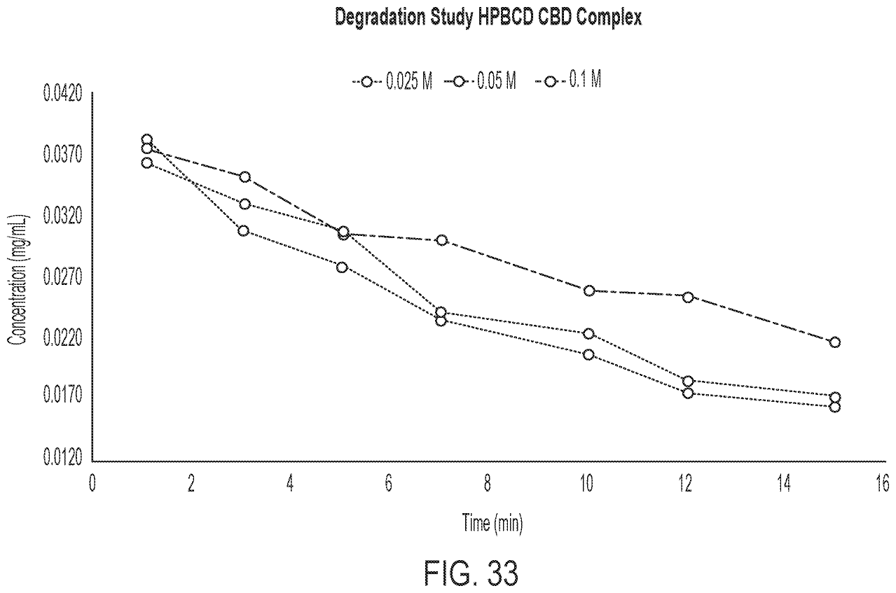

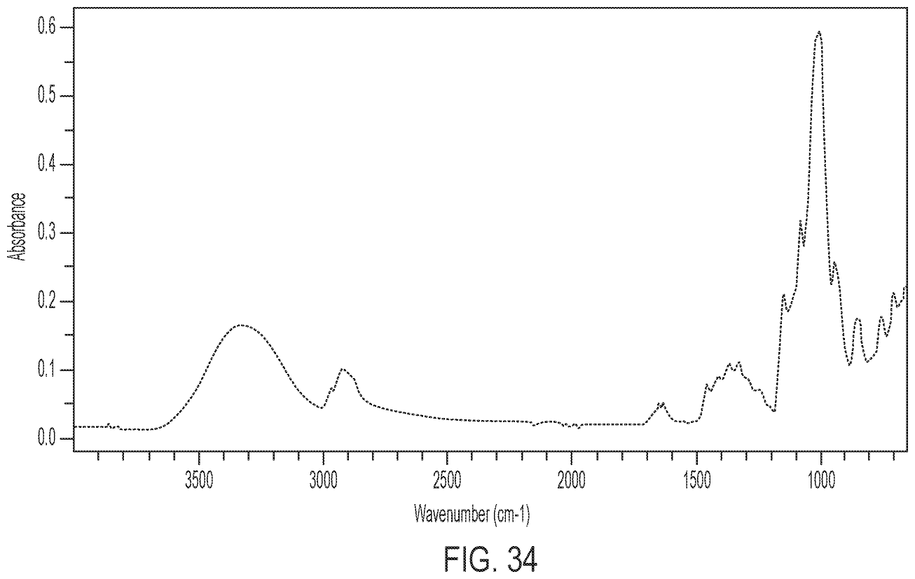

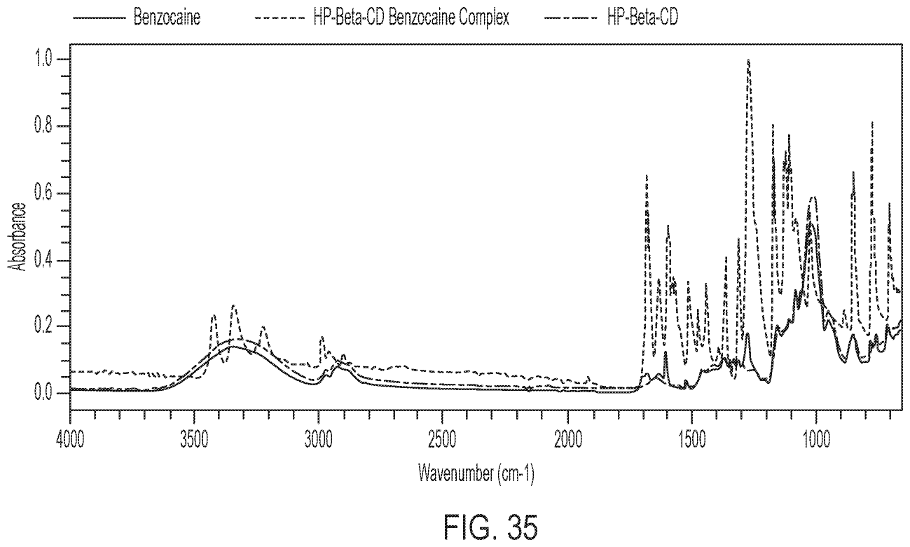

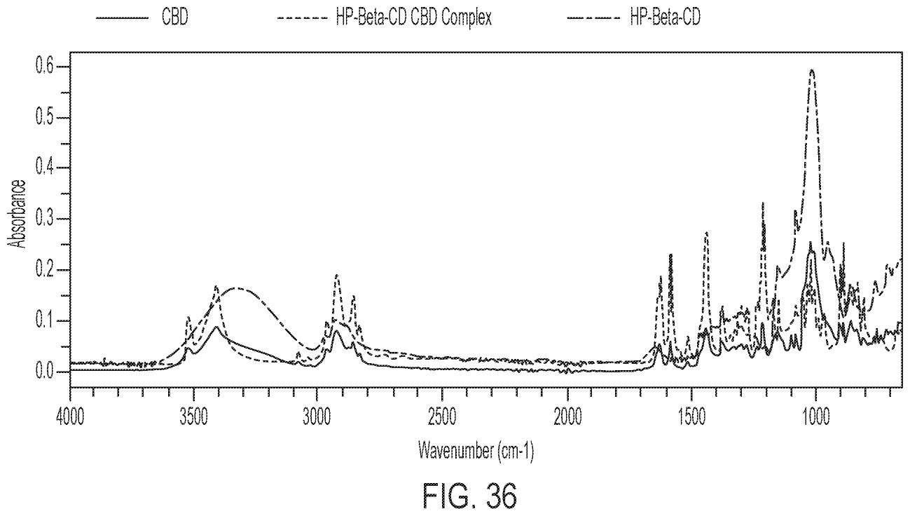

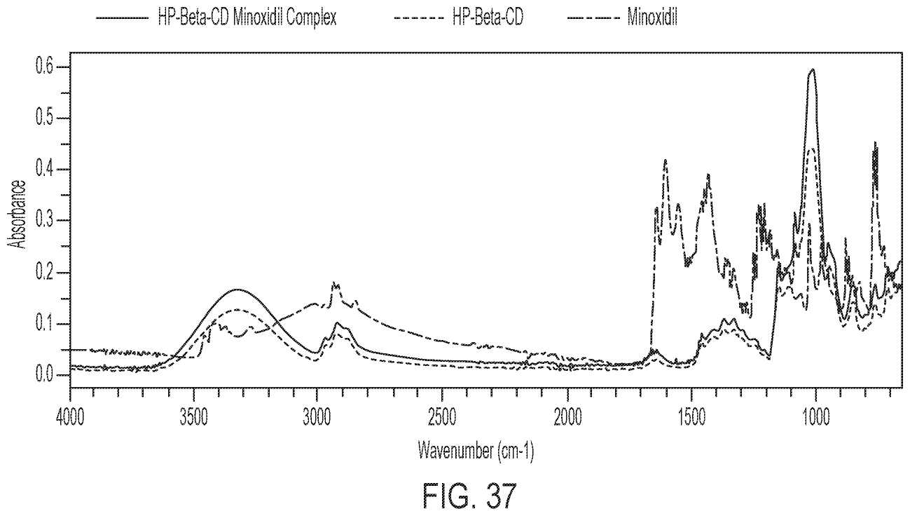

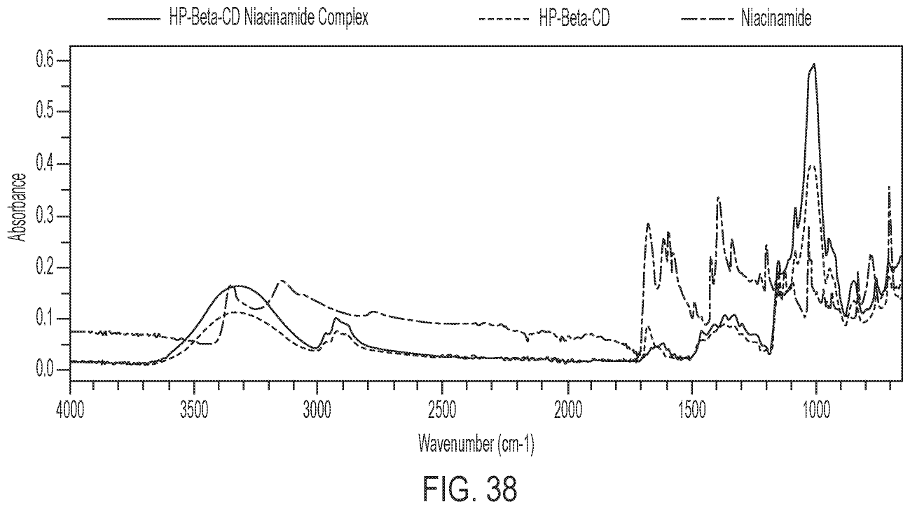

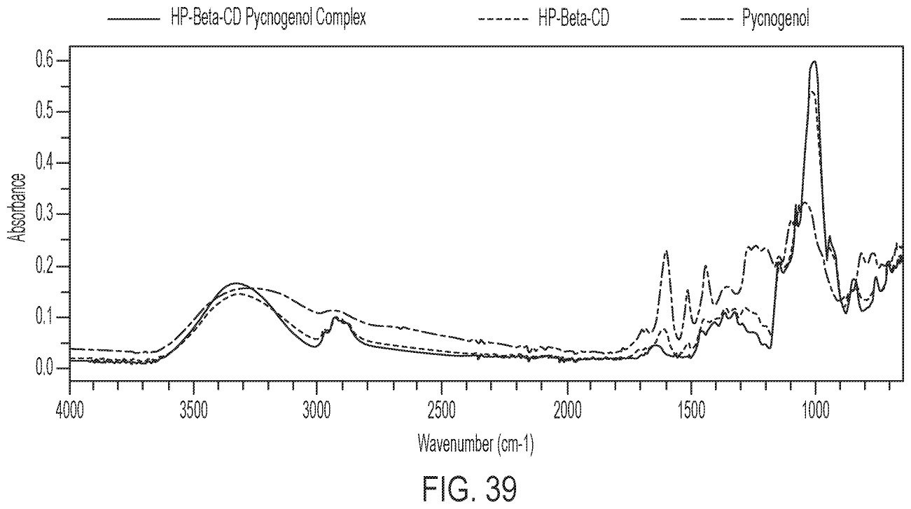

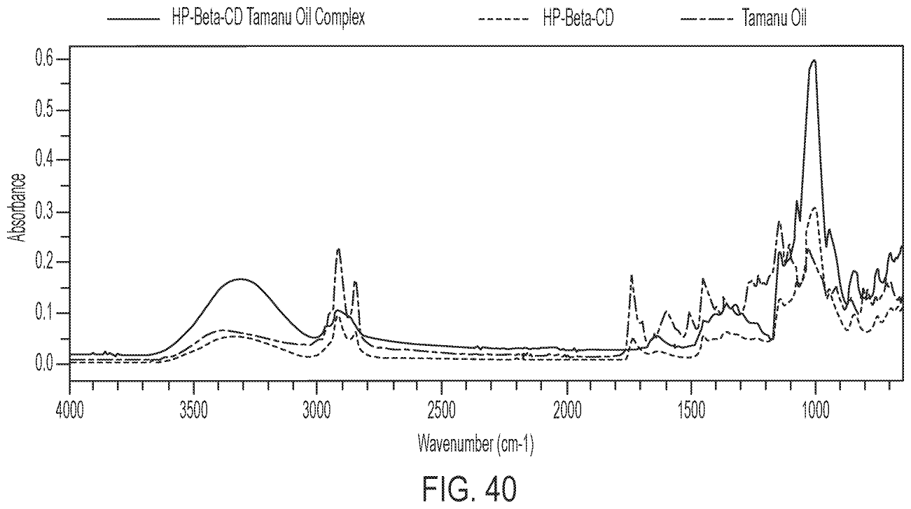

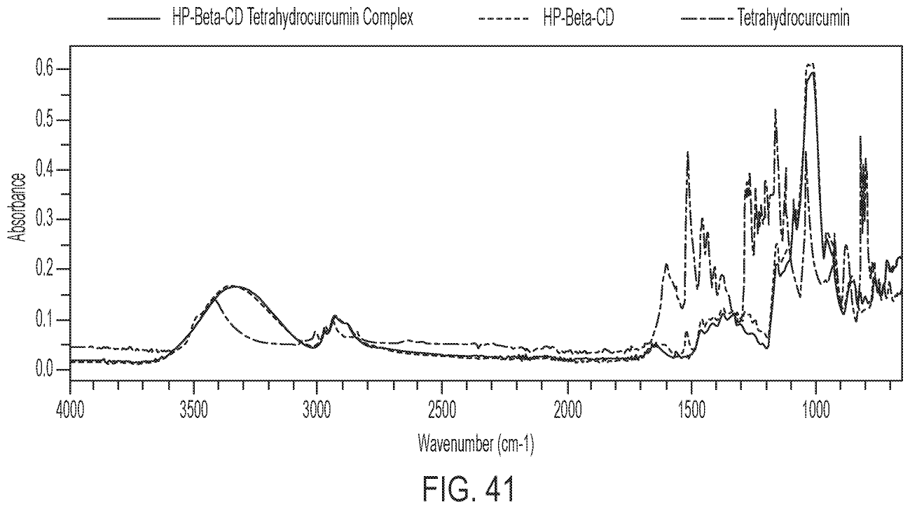

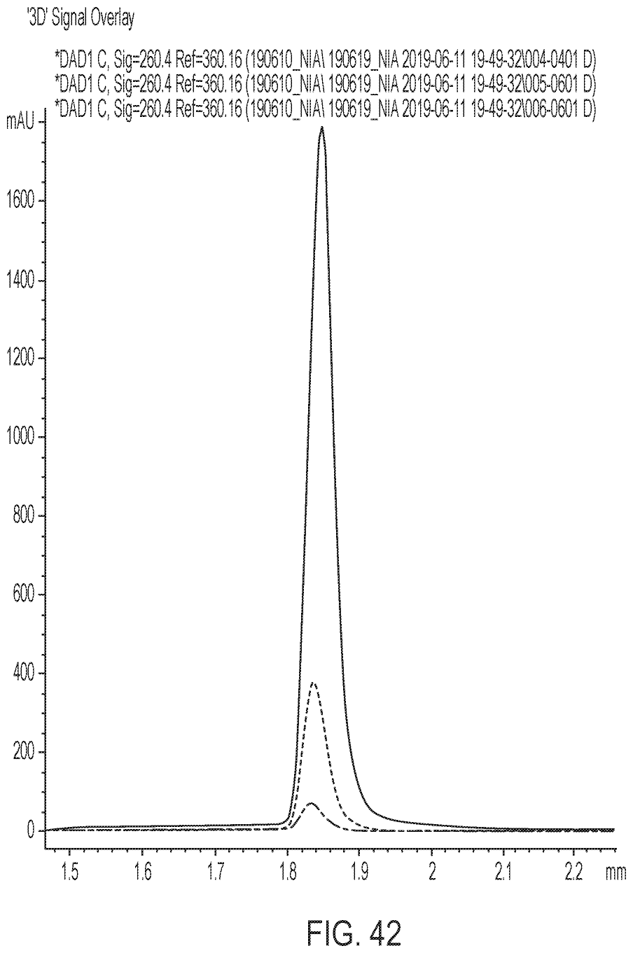

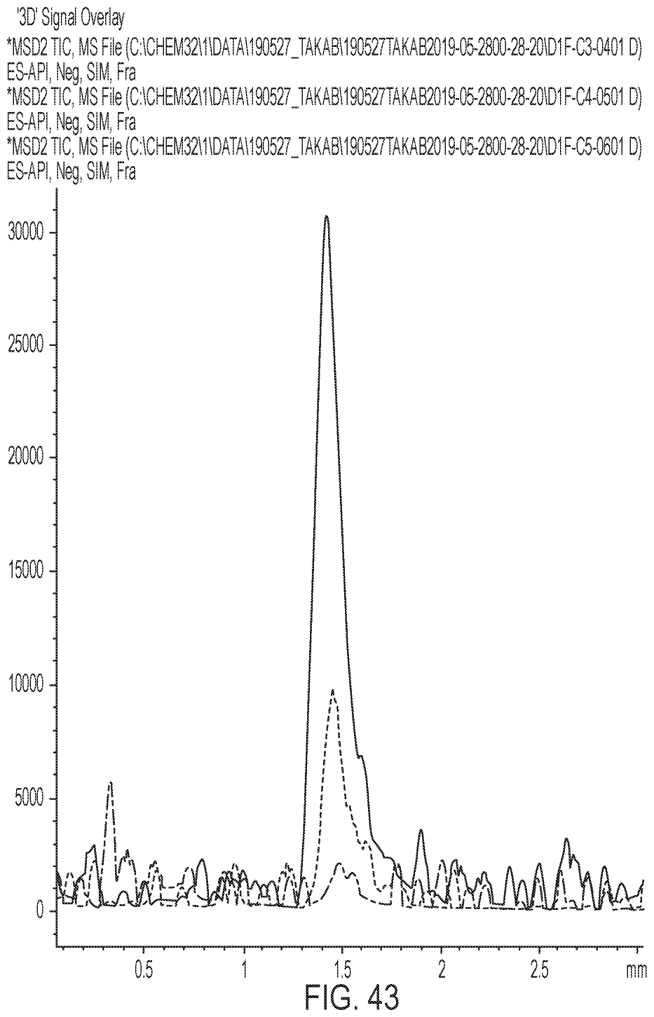

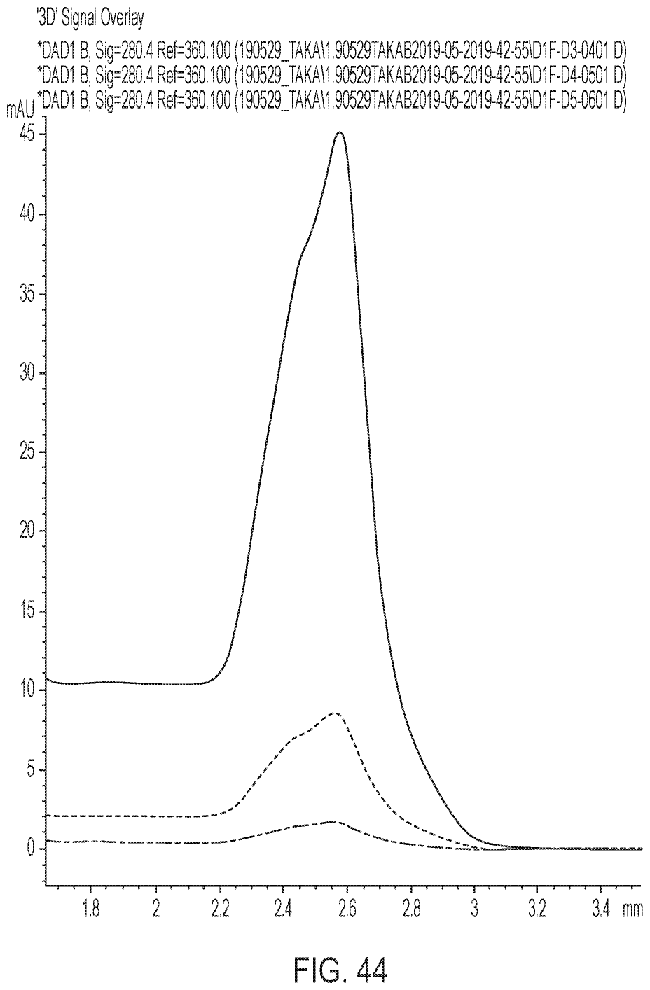

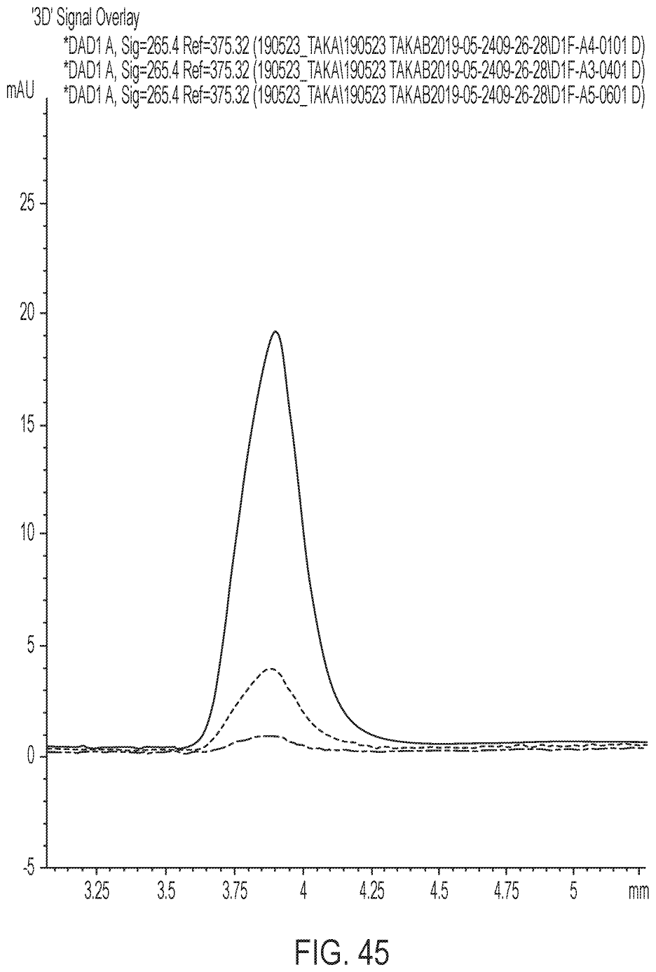

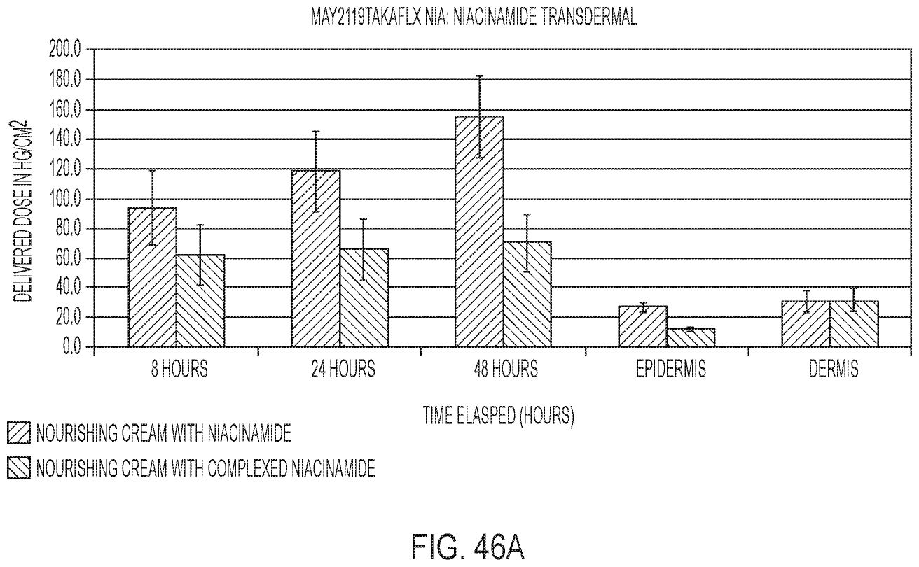

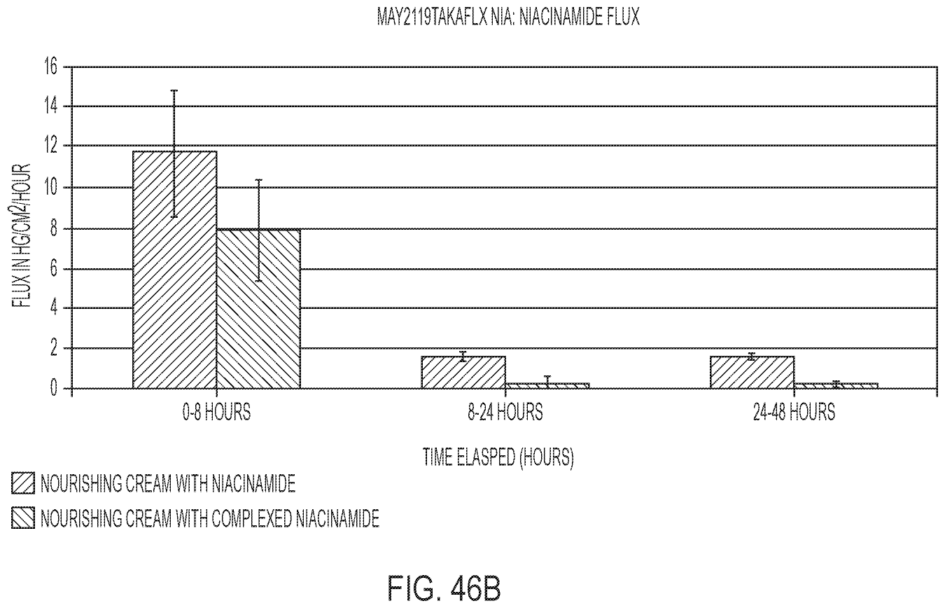

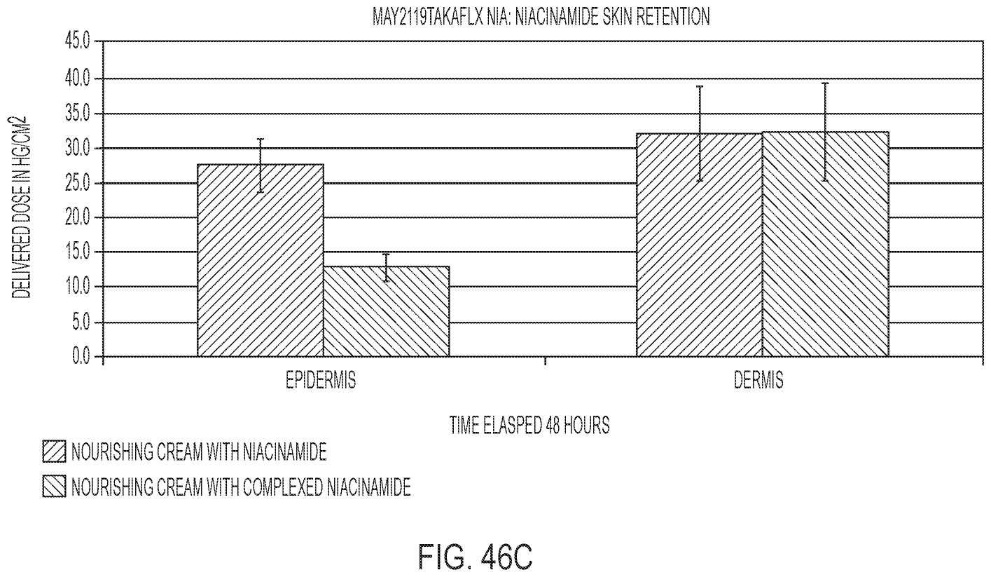

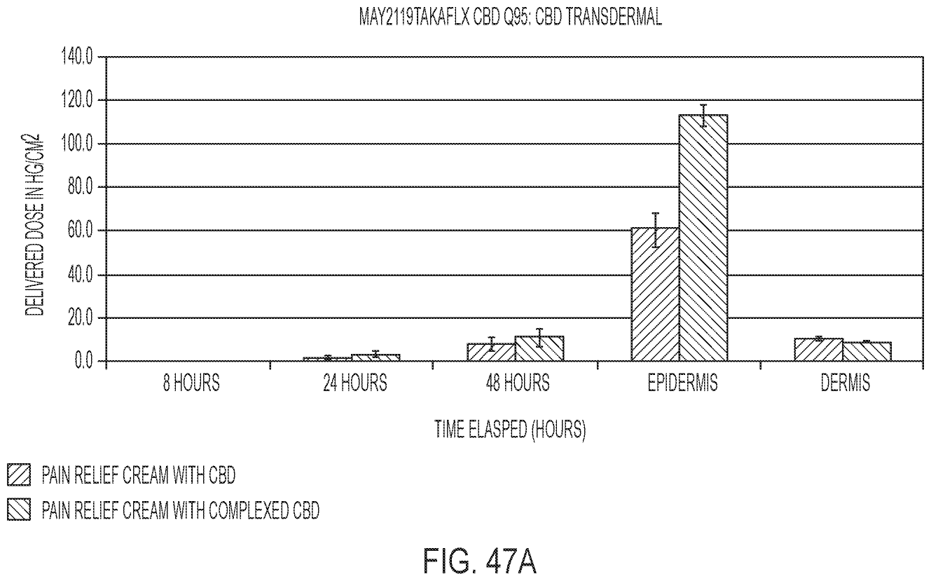

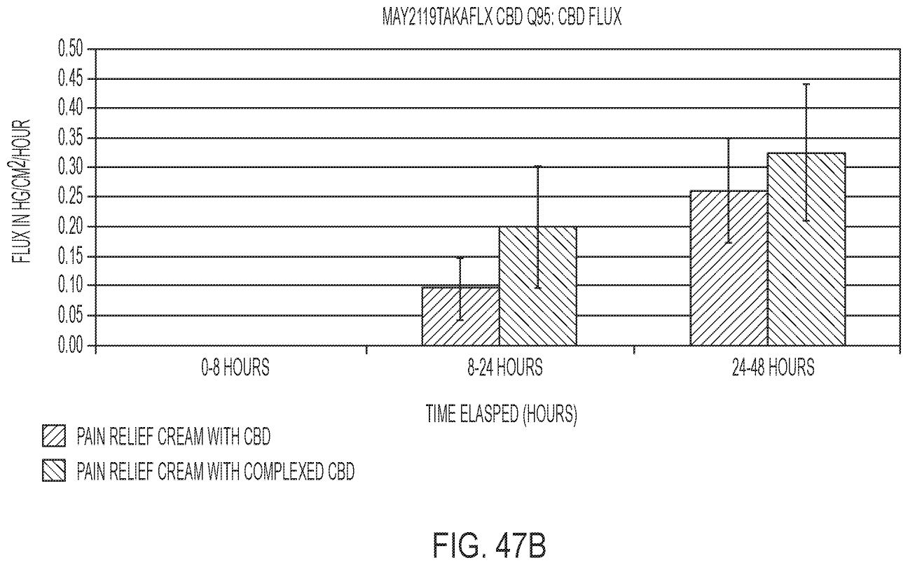

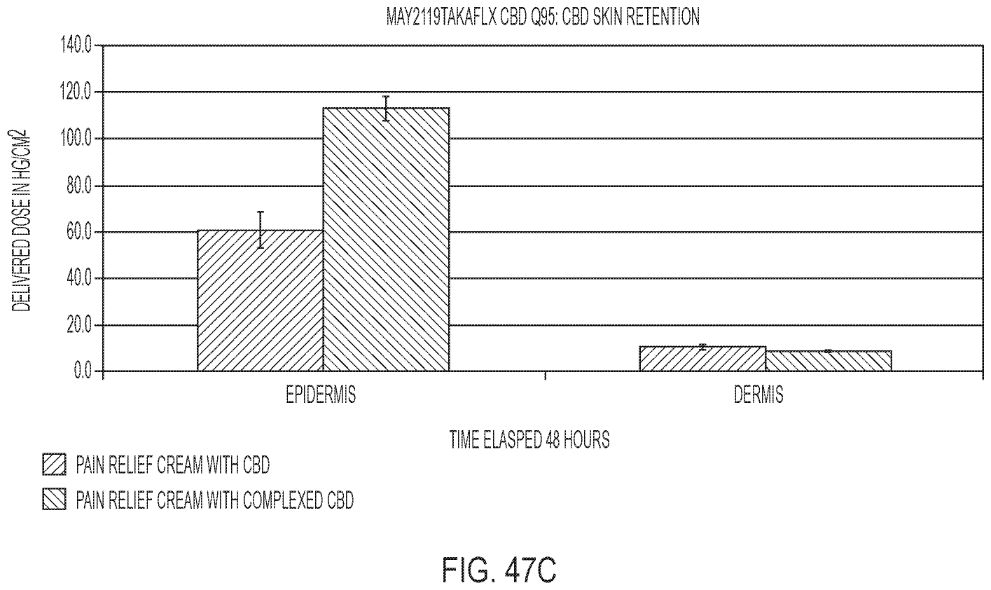

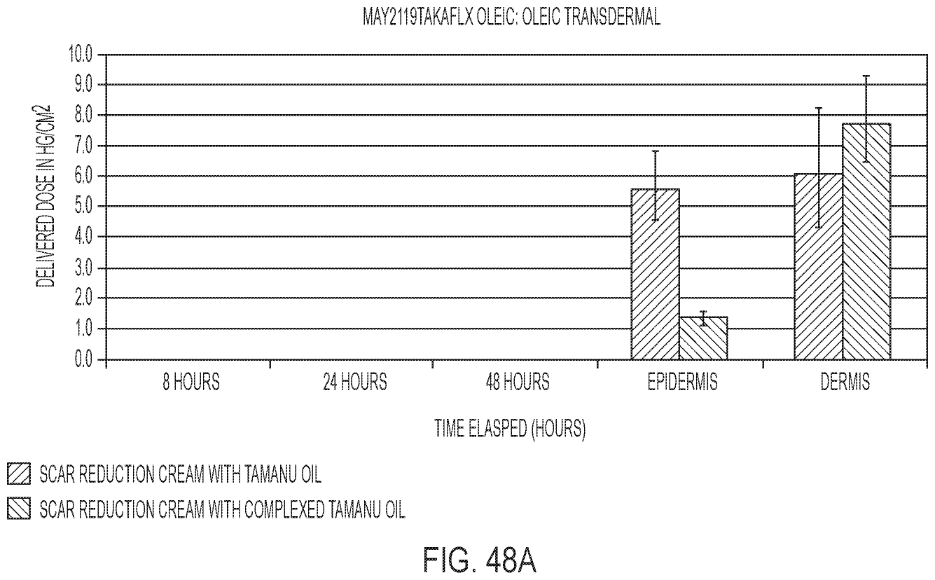

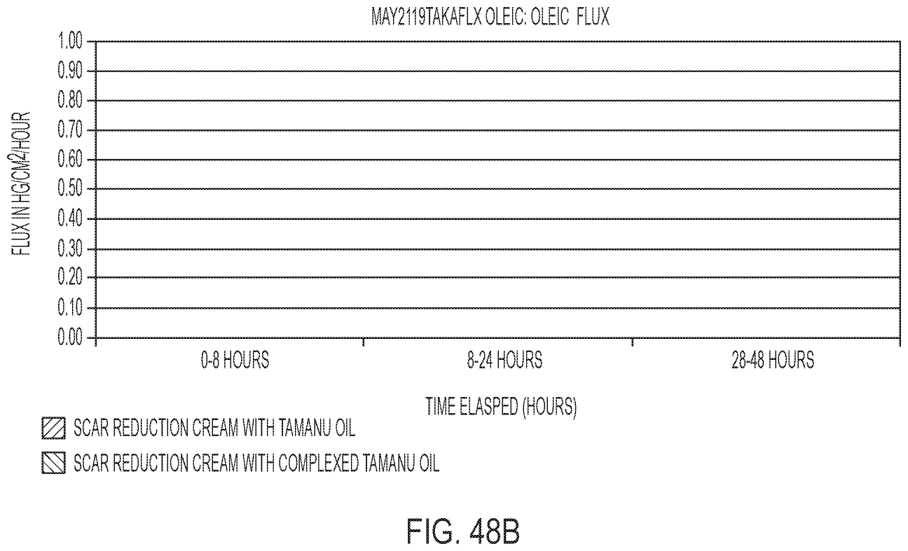

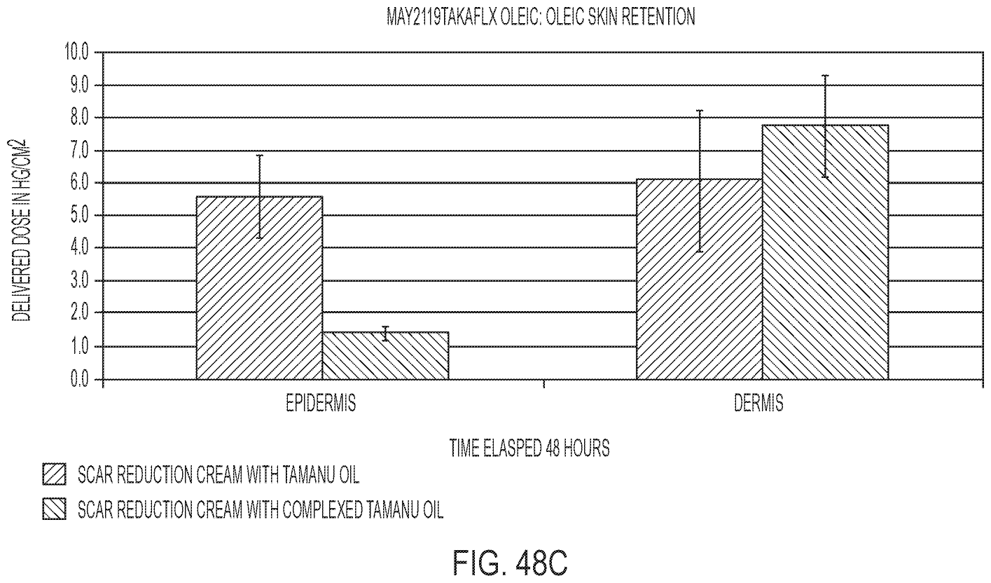

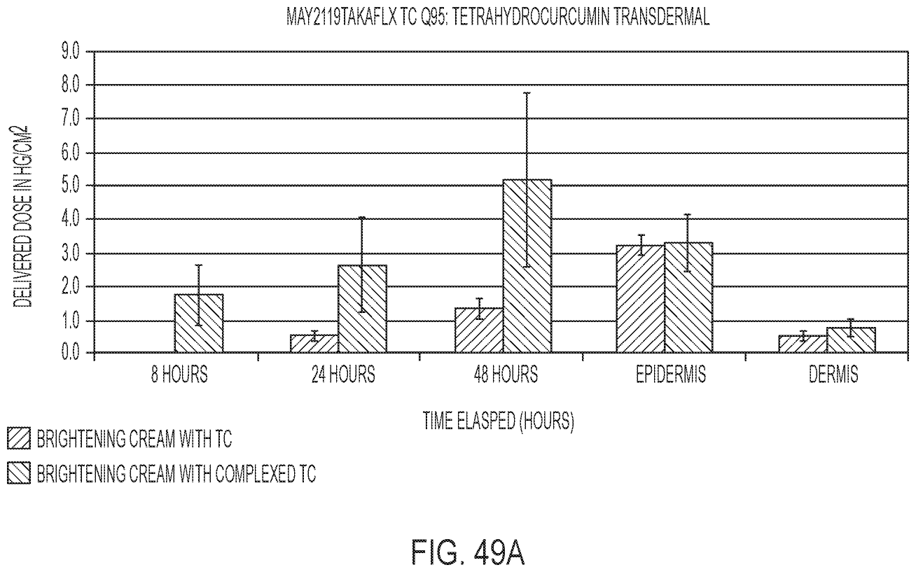

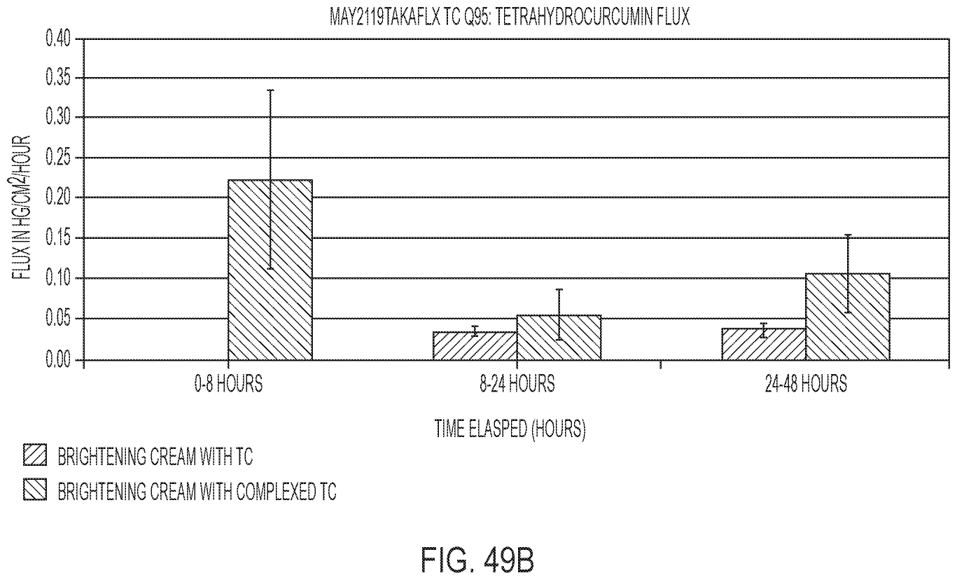

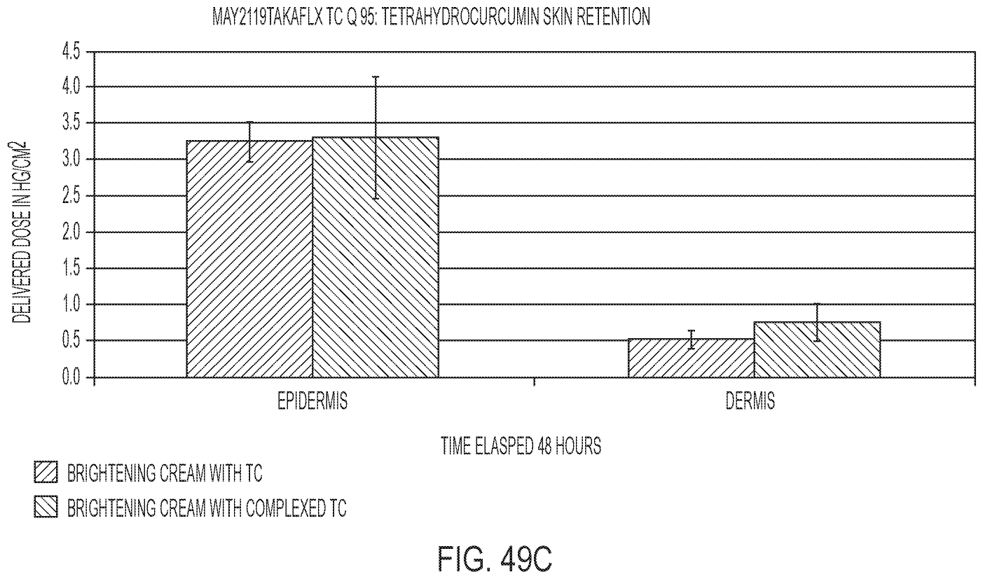

Other layers of the epidermis below the stratum corneum include the stratum lucidum, stratum granulosum, stratum germinativum, and stratum basale. Each contains living cells with specialized functions (FIG. 2). For example melanin, which is produced by melanocytes in the epidermis, is responsible for the color of the skin. Langerhans cells are involved in immune processing.

Dermal appendages, which include hair follicles, sebaceous and sweat glands, fingernails, and toenails, originate in the epidermis and protrude into the dermis hair follicles and sebaceous and sweat glands contribute epithelial cells for rapid reepithelialization of wounds that do not penetrate through the dermis (termed partial-thickness wounds). The sebaceous glands are responsible for secretions that lubricate the skin, keeping it soft and flexible. They are most numerous in the face and sparse in the palm of the hands and soles of the feet. Sweat gland secretions control skin pH to prevent dermal infections. The sweat glands, dermal blood vessels, and small muscles in the skin (responsible for goose pimples) control temperature on the surface of the body. Nerve endings in the skin include receptors for pain, touch, heat, and cold. Loss of these nerve endings increases the risk for skin breakdown by decreasing the tolerance of the tissue to external forces.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis and the deep dermis.

The superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts. The structural integrity of the dermis plays a role in the normal function and youthful appearance of the skin.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness. Dermal wounds, e.g., cracks or pustules, involve the epidermis, basal membrane, and dermis. Typically, dermal injuries heal rapidly.

Substances are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations.

The terms “soluble” and “solubility” refer to the property of being susceptible to being dissolved in a specified fluid (solvent). The term “insoluble” refers to the property of a material that has minimal or limited solubility in a specified solvent. In a solution, the molecules of the solute (or dissolved substance) are uniformly distributed among those of the solvent. A “suspension” is a dispersion (mixture) in which a finely-divided species is combined with another species, with the former being so finely divided and mixed that it doesn't rapidly settle out. In everyday life, the most common suspensions are those of solids in liquid. Among the acceptable vehicles and solvents that may be employed are water, Ringer's solution, and isotonic sodium chloride solution. As used herein, the term “solubility” intends the solubility with reference to the total amount of compound (e.g., including the amount of compound in both complexed and non-complexed form).

According to the European Pharmacopoeia, the solubility of a compound in water in the range of 15 to 25° C. is defined as follows:

| | Solvent in mL per gram compound |
|---|---|
| Very readily soluble | <1 |
| Readily soluble | from 1 to 10 |
| Soluble | from >10 to 30 |
| Hardly soluble | from >30 to 100 |
| Poorly soluble | from >100 to 1,000 |
| Very poorly soluble | from >1,000 to 10,000 |
| Water-insoluble | >10,000 |

The term “solubilizing agents” as used herein refers to those substances that enable solutes to dissolve.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvate” as used herein refers to a complex formed by the attachment of solvent molecules to that of a solute.

The term “solvent” as used herein refers to a substance capable of dissolving another substance (termed a “solute”) to form a uniformly dispersed mixture (solution).

The term “split thickness skin” as used herein refers to skin containing the epidermis and part of the dermis.

The term “stability” and its various grammatical forms as used herein refers to the capability of a particular formulation to remain within its physical, chemical, microbiological, therapeutic and toxicological specifications.

Unless otherwise stated, “substantially pure” in reference to an inclusion complex intends a preparation of the inclusion complex that contains about or less than about 15% impurity, wherein the impurity intends a compound other than an inclusion complex of a compound and the HPBCD. Substantially pure preparations include preparations that contain less than about 15% impurity, such as preparations that contain less than about any one of 15%, 12%, 10%, 8%, 5%, 3%, 2%, 1% and 0.5% impurity.

The term “substituted” as used herein refers to replacement of one element or radical by another as a result of a chemical reaction. A “substituent” is an atom or radical that replaces another in a molecule as a result of a chemical reaction. For the described invention, multiple degrees of substitution are contemplated unless otherwise stated.

The term “surfactant” or “surface-active agent” as used herein refers to an agent, usually an organic chemical compound that is at least partially amphiphilic, i.e., typically containing a hydrophobic tail group and hydrophilic polar head group. Surfactants generally are classified according to the nature of the hydrophilic group. Alternatively, HLB (Hydrophile-Lipophile Balance), an empirical expression for the relationship of the hydrophilic (“water-loving”) and hydrophobic (“water-hating”) groups of a surfactant, is the percentage weight of the hydrophilic group divided by 5 in order to reduce the range of values. The higher the HLB value, the more water-soluble the surfactant. For example, on a molar basis, e.g., a 100% hydrophilic molecule (e.g., polyethylene glycol) would have an HLB value of 20. An increase in polyoxyethylene chain length, which increases polarity, increases the HLB value; at constant polar chain length, an increase in alkyl chain length or number of fatty acid groups decreases polarity and the HLB value. Water-in-oil emulsions (w/o) require low HLB surfactants. Oil-in-water (o/w) emulsions often require higher HLB surfactants. For example, Triton-X45 has an HLB value of 9.8; but it is dispersible (not soluble) in water, while a blend of Triton X-35 (HLB=7.8) and Triton X-100 (HLB=13.4) will be water soluble. HLB values are additive; to achieve the required HLB value, the weighted average of the HLB values for each surfactant can be used.

The term “susceptible” as used herein refers to being at risk for.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “symptom” as used herein refers to a phenomenon that arises from and accompanies a particular disease or disorder and serves as an indication of it.

The term “technical grade” as used herein, with respect to excipients refers to excipients that may differ in specifications and/or functionality, impurities, and impurity profiles.

As used herein, the term “therapeutic agent” or “active agent” refers to the ingredient, component or constituent of the compositions of the described invention responsible for the intended therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction, or elimination of the progression of a disease manifestation.

The term “topical” as used herein refers to administration of an inventive composition at, or immediately beneath, the point of application. The term “topical administration” and “topically applying” as used herein are used interchangeably to refer to delivering a CD inclusion complex onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

Substances generally are applied to the skin to elicit one or more of four general effects: an effect on the skin surface, an effect within the stratum corneum, an effect requiring penetration into the epidermis and dermis, or a systemic effect resulting from delivery of sufficient amounts of a given substance through the epidermis and the dermis to the vasculature to produce therapeutic systemic concentrations. One example of an effect on the skin surface is formation of a film. Film formation may be protective (e.g., sunscreen) and/or occlusive (e.g., to provide a moisturizing effect by diminishing loss of moisture from the skin surface). One example of an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into or binding with skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

Medically, topical means applied to the surface of the skin or some other surface —many topical medications are epicutaneous, meaning that they are applied directly to the skin. Topical medications may also be inhalational, such as asthma medications, or applied to the surface of tissues other than the skin, such as eye drops applied to the conjunctiva, ear drops placed in the ear, or medications applied to the surface of a tooth.

The term “transdermal flux” as used herein refers to the rate of absorption of a substance across the dermal barrier. The flux is proportional to the concentration difference across the barrier.

The terms “treat,” “treated,” or “treating” as used herein refers to both therapeutic treatment and/or prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s). Treatment includes eliciting a clinically significant response without unacceptable levels of side effects. Treatment also includes prolonging survival as compared to expected survival if not receiving treatment.

The term “van der Waals forces” as used herein refers to relatively weak electric forces that attract neutral molecules to one another in gases, in liquefied and solidified gases, and in almost all organic liquids and solids.

The term “viscosity” as used herein refers to the property of a fluid that resists the force tending to cause the fluid to flow. Viscosity is a measure of the fluid's resistance to flow. The resistance is caused by intermolecular friction exerted when layers of fluids attempt to slide by one another. Viscosity can be of two types: dynamic (or absolute) viscosity and kinematic viscosity. Absolute viscosity or the coefficient of absolute viscosity is a measure of the internal resistance. Dynamic (or absolute) viscosity is the tangential force per unit area required to move one horizontal plane with respect to the other at unit velocity when maintained a unit distance apart by a fluid. Dynamic viscosity is usually denoted in poise (P) or centipoise (cP), wherein 1 poise=1 g/cm², and 1 cP=0.01 P. Kinematic viscosity is the ratio of absolute or dynamic viscosity to density. Kinematic viscosity is usually denoted in Stokes (St) or Centistokes (cSt), wherein 1 St=10-4 m²/s, and 1 cSt=0.01 St.

As used herein, a “wt %” or “weight percent” or “percent by weight” or “wt/wt %” of a component, unless specifically stated to the contrary, refers to the ratio of the weight of the component to the total weight of the composition in which the component is included, expressed as a percentage.

### CDs and CD Inclusion Complexes

According to some embodiments of the invention, the cyclodextrin for use in the inclusion complexes and formulations herein is a water soluble unsubstituted or substituted beta-cyclodextrin (BCD). According to some embodiments, the beta-cyclodextrin is selected from the group consisting of methyl beta-cyclodextrin (MBCD), hydroxypropyl beta-cyclodextrin (HPBCD), and sulfobutylether beta-cyclodextrin (SBEBCD). According to some embodiments, the beta-cyclodextrin is hydroxypropyl beta-cyclodextrin. According to some embodiments, the beta-cyclodextrin is a substituted hydroxypropyl beta-cyclodextrin. According to some embodiments, mixtures of cyclodextrins may also be employed. For example, a formulation comprising an active compound and a mixture of two or three or four or more cyclodextrins is also provided.

According to some embodiments, the cyclodextrin can be obtained from a commercial source, including, but not limited to cyclodextrins sold under the following tradenames CAVASOL® W6 HP (Wacker Chemic AG, Munich, Germany), CAVASOL® W6 HP TL (Wacker Chemie AG, Munich, Germany), CAVAMAX® W6 Pharma (Wacker Chemie AG, Munich, Germany), CAVASOL® W7 HP (Wacker Chemie AG, Munich, Germany), CAVASOL® W7 HP Pharma (Wacker Chemic AG, Munich, Germany), CAVASOL® W7 HP TL (Wacker Chemie AG, Munich, Germany), CAVASOL W7 M (Wacker Chemie AG, Munich, Germany), CAVASOL® W7 M Pharma (Wacker Chemie AG, Munich, Germany), CAVASOL® W7 M TL (Wacker Chemie AG, Munich, Germany), CAVASOL® W8 HP (Wacker Chemie AG, Munich, Germany), CAVASOL® W8 HP Pharma (Wacker Chemie AG, Munich, Germany), KLEPTOSE® HPB (Roquette Pharma, Geneva, Ill.), and CAPTISOL® (Cyclex Pharmaceuticals, Inc. Lenexa, Kans.).

Exemplary classes of small molecule compounds include, without limitation: an anti-fungal agent, an anti-histamine agent; an anti-hypertensive agent; an anti-protozoal agent; an anti-oxidant; an anti-pruritic agent; an anti-skin atrophy agent; an anti-viral agent; a caustic agent; a calcium channel blocker; a cytokine-modulating agent; a prostaglandin analog; a chemotherapeutic agent; an irritant agent; a TRPC channel inhibitor agent; and a vitamin.

The term “anti-fungal agent” as used herein means any of a group of chemical substances having the capacity to inhibit the growth of or to destroy fungi. Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, B iphenamine, Bromo salicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “imidazole” (1,3-diazacyclopenta-2,4-diene) refers to a five-membered aromatic heterocycle having the following structure:

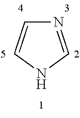

It exists in two equivalent tautomeric forms due to a hydrogen atom that may be located on either of the two nitrogen atoms.

The N-3 nitrogen atom of imidazole, which possesses a non-bonding pair of electrons, is unusually basic for an sp2-hybridized nitrogen atom. Its conjugate acid, which is called an imidazolium ion and is stabilized by resonance, has a pKa of approximately 7.0, as depicted below. Consequently, imidazole readily interconverts between its conjugate base and conjugate acid forms under physiological conditions, i.e. aqueous conditions near neutral pH. Furthermore, imidazole's Lewis basicity, which can be enhanced by complete or partial deprotonation of N-1, makes it an excellent ligand for many metal ions, including those that occur in biological systems.

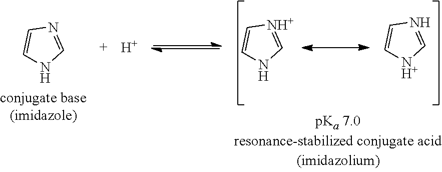

Histidine, one of the 20 endogenous amino acids that are most commonly found in proteins, contains an imidazole ring in its sidechain, which exhibits the moderate basicity and affinity for metals ions described above for imidazole itself. Due to these properties, histidine residues are essential for the normal function of many enzymes, receptors and other proteins. For example, histidine residues serve as facilitators of proton transfer in the active sites of many enzymes. Histidine residues also play several key roles in the cooperative binding and release of oxygen by hemoglobin. Decarboxylation of histidine affords histamine, an important neurotransmitter in which the imidazole moiety is essential for binding to histamine receptors.

Synthetic imidazoles are present in many fungicides, antiprotozoal and antihypertensive agents. Imidazole also is part of the theophylline molecule, found in tea leaves and coffee beans, and stimulates the central nervous system. A preservative system for ophthalmic solutions comprising imidazole and a hydrogen peroxide source has been shown to be effective against fungi and bacteria (U.S. Pat. No. 6,565,894).

Examples of known imidazoles include, but are not limited to, histidines, the antimicrobial agents bifonazole, butoconazole, chlorimidazole, hlordantoin, croconazole, clotrimazole, democonazole, eberconazole, econazole, elubiol, enilconazole, fenticonazole, flutrimazole, isocanazole, ketoconazole, lanoconazole, lombazole, miconazole, neticonazole, NND-502, omoconazole, oxiconazole, parconazole, sertaconazole, sulconazole, tiabendazole, and tioconazole, and the thromboxane synthase inhibitors 7-(1-imidazolyl)hepatanoic acid, ozagrel, and 1-benzyl imidazole.

Other nitrogen-containing 5-membered aromatic heterocycles can be considered analogs of imidazole. The term “imidazole analogs” is used herein to describe imidazoles and related 5-membered aromatic heterocycles that contain at least two nitrogen atoms in the ring. Such heterocycles are exemplified, but not limited to, 1,2,4-triazole, 1,3,4-triazole, 1,2,3-triazole, tetrazole and pyrazole, as well as thiadiazoles and oxadiazoles. Several triazoles are useful, particularly as fungicides, including albaconazole, CAS RN 214543-30-3, fluconazole, genaconzole, hydroxyitraconazole, isavuconazole, itraconazole, pramiconazole, ravuconazole, saperconazole, SYN 2869, T 8581, TAK 456, terconazole, vibunazole, voriconazole, pramiconazole, and posaconazole.

Miconazole, for example, which commonly is applied topically to the skin or to mucus membranes to treat fungal infections, such as athlete's foot and jock itch, and for vaginal yeast infections, is commercially available as a cream, lotion, powder, spray liquid, and spray powder for skin applications. Miconazole is an imidazole of the structure:

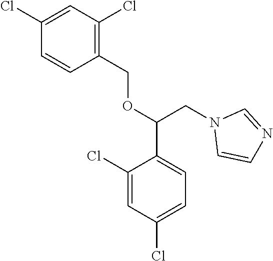 [SMILES: Clc1ccc(COC(Cn2ccnc2)c2ccc(Cl)cc2Cl)c(Cl)c1]

Miconazole's antifungal activity (and that of the other azole antifungals) is believed to be due to inhibition of ergosterol synthesis, specifically by inhibiting the cytochrome P450-dependent lanosterol 14α-demethylase enzyme.

Ketoconazole, an imidazole anti-fungal agent having the structure:

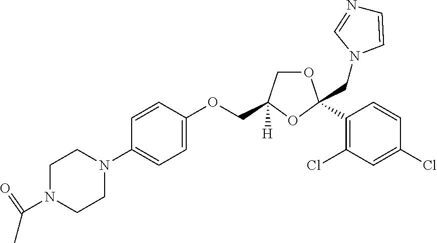 [SMILES: CC(=O)N1CCN(c2ccc(OC[C@H]3CO[C@](Cn4ccnc4)(c4ccc(Cl)cc4Cl)O3)cc2)CC1]

has been found to be effective in the treatment of seborrheic dermatitis. One open-label study of minoxodil 2% with ketoconazole 2% shampoo for androgenetic alopecia in men reportedly showed comparable growth in both groups, with both achieving better growth than unmedicated shampoo alone. Similar results were seen in a mouse model comparing topical ketoconazole 2% to a placebo. Ketoconazole also has been used to treat hirsutism in women, with some success. The mechanism of action is not understood.

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine

Antihypertensive Agents:

Blood pressure is the force of blood pushing against the wall of the arteries as your heart pumps out blood into the arteries. Its level varies with age, sex, level of physical activity and emotional changes. The term “hypertension” as used herein refers to high systemic blood pressure; transitory or sustained elevation of systemic blood pressure to a level likely to induce cardiovascular damage or other adverse consequences. According to the World Health Organization, “hypertension” is defined as systolic/diastolic pressure persistently higher than 140/90 mmHg. anti-hypertensive agents are used to lower high blood pressure. There are many different types of antihypertensive agents, and they work in different ways to lower blood pressure. Non-limiting examples include, without limitation, ACE inhibitors (e.g. enalapril, lisinopril, perindopril); Angiotensin II receptor blockers (e.g. losartan, valsartan); calcium channel blockers (see supra); Diuretics (e.g. amiloride, frusemide, indapamide); Beta-blockers (e.g. atenolol, metoprolol, propranolol); Alpha-blockers (e.g., doxazosin, prazosin); Centrally acting antihypertensive drugs (e.g., methyldopa, clonidine); Vasodilators (e.g., hydralazine, minoxidil (Loniten®)).

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®) sulfadiazine, and Leucovorin.

The term “antipruritic agents” as used herein refers to those substances that reduce, eliminate or prevent itching. Antipruritic agents include, without limitation, pharmaceutically acceptable salts of methdilazine and trimeprazine.

The term “anti-oxidant agent” as used herein refers to a substance that inhibits oxidation or reactions promoted by oxygen or peroxides. Non-limiting examples of antioxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename TroloxR), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione, N-acetylcysteine and its derivatives), dihydroxy fumaric acid and its salts, glycine pidolate, arginine pilolate, nordihydroguaiaretic acid, bioflavonoids, olyphenols, e.g., resveratrol, and its analogs, e.g., trans-reveratrol. curcumin, lysine, methionine, proline, superoxide dismutase, silymarin, tea extracts, grape skin/seed extracts, melanin, and rosemary extracts.

The term “anti-skin atrophy actives” refers to substances effective in replenishing or rejuvenating the epidermal layer by promoting or maintaining the natural process of desquamation. Non-limiting examples of antiwrinkle and antiskin atrophy actives, which can be used in context of the described invention, include retinoic acid, its prodrugs and its derivatives (e.g., cis and trans) and analogues; salicylic acid and derivatives thereof, sulfur-containing D and L amino acids (e.g., cysteine, methionine) and their derivatives (e.g., N-acetylcysteine) and salts; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “caustic agents” as used herein refers to substances capable of destroying or eating away epithelial tissue by chemical action. Caustic agents can be used to remove dead skin cells. For example, beta-hydroxy acids, naturally derived acids with a strong keratolytic effect, are useful for problem skin, acne or peeling.

Calcium Channel Blockers.

Calcium channel blockers act upon voltage-gated calcium channels (VGCCs) in muscle cells of the heart and blood vessels. By blocking the calcium channel they prevent large increases of the calcium levels in the cells when stimulated, which subsequently leads to less muscle contraction. In the heart, a decrease in calcium available for each beat results in a decrease in cardiac contractility. In blood vessels, a decrease in calcium results in less contraction of the vascular smooth muscle and therefore an increase in blood vessel diameter. The resultant vasodilation decreases total peripheral resistance, while a decrease in cardiac contractility decreases cardiac output. Since blood pressure is in part determined by cardiac output and peripheral resistance, blood pressure drops.

Calcium channel blockers do not decrease the responsiveness of the heart to input from the sympathetic nervous system. Since blood pressure regulation is carried out by the sympathetic nervous system (via the baroreceptor reflex), calcium channel blockers allow blood pressure to be maintained more effectively than do β-blockers. However, because calcium channel blockers result in a decrease in blood pressure, the baroreceptor reflex often initiates a reflexive increase in sympathetic activity leading to increased heart rate and contractility. The decrease in blood pressure also likely reflects a direct effect of antagonism of VDCC in vascular smooth muscle, leading to vasodilation. A β-blocker may be combined with a calcium channel blocker to minimize these effects.

L-type VDCC inhibitors are calcium entry blocking drugs whose main pharmacological effect is to prevent or slow entry of calcium into cells via L-type voltage-gated calcium channels. Examples of L-type calcium channel inhibitors include but are not limited to: dihydropyridine L-type blockers such as nisoldipine, nicardipine and nifedipine, AHF (such as 4aR,9aS)-(+)-4a-Amino-1,2,3,4,4a,9a-hexahydro-4aH-fluorene, HCl), isradipine (such as 4-(4-Benzofurazanyl)-1,-4-dihydro-2,6-dimethyl-3,5-pyridinedicarboxylic acid methyl 1-methhylethyl ester), Calciseptin/calciseptine (such as isolated from (Dendroaspis polylepis polylepis), Cilnidipine (such as also FRP-8653, a dihydropyridine-type inhibitor), Dilantizem (such as (2S,3 S)-(+)-cis-3-Acetoxy-5-(2-dimethylaminoethyl)-2,3-dihydro-2-(4-methoxyphenyl)-1,5-benzothiazepin-4(5H)-one hydrochloride), diltiazem (such as benzothiazepin-4(5H)-one, 3-(acetyloxy)-5-[2-(dimethylamino)ethyl]-2,3-dihydro-2-(4-methoxyphenyl)-, (+)-cis-, monohydrochloride), Felodipine (such as 4-(2,3-Dichlorophenyl)-1,4-dihydro-2,6-dimethyl-3,5-pyridinecarboxylic acid ethyl methyl ester), FS-2 (such as an isolate from Dendroaspis polylepis polylepis venom), FTX-3.3 (such as an isolate from Agelenopsis aperta), Neomycin sulfate (such as C₂₃H₄₆N_{60.13.3}H₂SO₄), Nicardipine (such as 1,4-Dihydro-2,6-dimethyl-4-(3-nitrophenyl)methyl-2-[methyl(phenylmethyl) a-mino]-3,5-pyridinedicarboxylic acid ethyl ester hydrochloride, also YC-93, Nifedipine (such as 1,4-Dihydro-2,6-dimethyl-4-(2-nitrophenyl)-3,5-pyridinedicarboxylic acid dimethyl ester), Nimodipine (such as 4-Dihydro-2,6-dimethyl-4-(3-nitrophenyl)-3,5-pyridinedicarboxylic acid 2-methoxyethyl 1-methylethyl ester) or (Isopropyl 2-methoxyethyl 1,4-dihydro-2,6-dimethyl-4-(m-nitrophenyl)-3,5-pyridinedicarboxylate), Nitrendipine (such as 1,4-Dihydro-2,6-dimethyl-4-(3-nitrophenyl)-3,5-pyridinedicarboxylic acid ethyl methyl ester), S-Petasin (such as (3 S,4aR,5R,6R)-[2,3,4,4a,5,6,7,8-Octahydro-3-(2-propenyl)-4a,5-dimethyl-2-o-xo-6-naphthyl]Z-3′-methylthio-1′-propenoate), Phloretin (such as 2′,4′,6′-Trihydroxy-3-(4-hydroxyphenyl)propiophenone, also 3-(4-Hydroxyphenyl)-1-(2,4,6-trihydroxyphenyl)-1-propanone, also b-(4-Hydroxyphenyl)-2,4,6-trihydroxypropiophenone), Protopine (such as C2OH19NO. 5Cl), SKF-96365 (such as 1-[b-[3-(4-Methoxyphenyl)propoxy]-4-methoxyphenethyl]-1H-imidazole, HCl), Tetrandine (such as 6,6′,7,12-Tetramethoxy-2,2′-dimethylberbaman), (.+−.)-Methoxyverapamil or (+)-Verapamil (such as 5-[N-(3,4-Dimethoxyphenylethyl)methylamino]-2-(3,4-dimethoxyphenyl)-2-iso-propylvaleronitrile hydrochloride), and (R)-(+)-Bay K8644 (such as R-(+)-1,4-Dihydro-2,6-dimethyl-5-nitro-4-[2-(trifluoromethyl)phenyl]-3-py-ridinecarboxylic acid methyl ester). The foregoing examples may be specific to L-type voltage-gated calcium channels or may inhibit a broader range of voltage-gated calcium channels, e.g. N, P/Q, R, and T-type.

Exemplary drugs for treating glaucoma, a group of eye conditions that can cause blindness, include, without limitation, Brimonidine/Timolol (ophthalmic alpha-2-agonist and ophthalmic beta blocker combination sold as Combigan®; Dorzolamide/timolol (beta blocker, sold as Cospot® for treating glaucoma); and Levobunolol (ophthalmic beta blocker, sold as Levobunolol® for glaucoma.

Prostaglandin analogs. Prostaglandins are a family of a group of lipid compounds that are derived enzymatically in the body from essential fatty acids. Every prostaglandin contains 20 carbon atoms, including a 5-carbon ring. Prostaglandins have a wide variety of effects, including, but not limited to, muscular constriction, mediating inflammation, calcium movement, hormone regulation and cell growth control. Prostaglandins act on a variety of cells, including vascular smooth muscle cells (causing constriction or dilation), platelets (causing aggregation or disaggregation), and spinal neurons (causing pain). Scientists stumbled on the hair thickening properties of prostaglandin F2a analogs while researching their use as an intraocular pressure (IOP)—lowering drug for use in patients with glaucoma and ocular hypertension. For example, latanoprost [(1R,2R, 3R, 5S)3,5-dihydroxy-2-[(3R)-3-hydroxy-5-phenylpentyl]cyclopentyl]-5-heptenoate], is marketed by Pfizer as Xalatan®. See U.S. Pat. No. 6,262,105, issued to Johnstone; bimatoprost (cyclopentane N-ethyl heptenamide-5-cis-2-(3α-hydroxy-5-phenyl-1-trans-pentenyl)-3,4-dihydroxy, [1α, 2β, 3α, 5α], is sold by Allergan, Inc. of Irvine, Calif. as Lumigan®, a 0.03% ophthalmic solution for treating glaucoma and as Latisse® to improve eyelash appearance when applied topically; isopropyl (Z)-7-[(1R,2R,3R,5S)-3,5-dihydroxy-2-[(1E,3R)-3-hydroxy-4-[(α,α,α-trifluoro-m-tolyfloxy]-1-butenyl]cyclopentyl]-5-heptenoate, or Travaprost (TRAVATAN® Alcon), is available as a 0.004% ophthalmic solution; The chemical name for tafluprost is 1-methylethyl (5Z)-7{(1R,2R,3R,5S)-2-[(1E)-3,3-difluoro-4-phenoxy-1-butenyl}-3,5-dihydroxycyclopentyl]-5-heptenoate. (Tafluprost, sold as Zioptan®), a fluorinated analog of prostaglandin F2a; and 16-phenoxy tetranor PGF2α cyclopropyl amide (see e.g., U.S. Pat. Nos. 7,645,800; 7,514,474; 7,649,021; 7,632,868; 7,517,912, incorporated herein by reference).

The term “chemotherapeutic agent” as used herein refers to chemicals useful in the treatment or control of a disease. Non-limiting examples of chemotherapeutic agents usable in context of the described invention include temozolomide, busulfan, ifosamide, melphalan, carmustine, lomustine, mesna, 5-fluorouracil, capecitabine, gemcitabine, floxuridine, decitabine, mercaptopurine, pemetrexed disodium, methotrexate, vincristine, vinblastine, vinorelbine tartrate, paclitaxel, docetaxel, ixabepilone, daunorubicin, epirubicin, doxorubicin, idarubicin, amrubicin, pirarubicin, mitoxantrone, etoposide, etoposide phosphate, teniposide, mitomycin C, actinomycin D, colchicine, topotecan, irinotecan, gemcitabine cyclosporin, verapamil, valspodor, probenecid, MK571, GF120918, LY335979, biricodar, terfenadine, quinidine, pervilleine A and XR9576.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. The drawbacks of cytokine therapy result from the basic properties of cytokines: (i) cytokines are pleiotropic, meaning that they affect several processes in parallel; (ii) cytokines are also known to have redundancy, meaning that the effects achieved by blocking one specific cytokine activity can be compensated by others (although this can be also beneficial, since a biological agent can be replaced to different cytokine blocker when incomplete remission or in case of intolerance); (iii) the cytokine network is a regulated and balanced system and its alteration may lead to impaired immune response. Exemplary cytokine modulating agents include, without limitation, etanercept; adalimumab; infloximab; certolizumab and golimumab (TNFα); Rilonacept; canakinumab (IL-1); Siltuximab (IL-6); Ustekinumab (IL-12 and IL-23); ixekizumab Secukinumab (IL-17, IL17A).

Transient receptor potential cation (TRPC) channels are widely expressed among cell types and may play important roles in receptor-mediated Ca2+ signaling. The TRPC3 channel is known to be a Ca2+-conducting channel activated in response to phospholipase C-coupled receptors. TRPC3 channels have been shown to interact directly with intracellular inositol 1,4,5-trisphosphate receptors (InsP3Rs) and that channel activation is mediated through coupling to InsP3Rs.

Agents useful for increasing arterial blood flow, inhibiting vasoconstriction or inducing vasodilation are agents that inhibit TRP channels. These inhibitors embrace compounds that are TRP channel antagonists. Such inhibitors are referred to as activity inhibitors or TRP channel activity inhibitors. As used herein, the term “activity inhibitor” refers to an agent that interferes with or prevents the activity of a TRP channel. An activity inhibitor may interfere with the ability of the TRP channel to bind an agonist such as UTP. An activity inhibitor may be an agent that competes with a naturally occurring activator of TRP channel for interaction with the activation binding site on the TRP channel. Alternatively, an activity inhibitor may bind to the TRP channel at a site distinct from the activation binding site, but in doing so, it may, for example, cause a conformational change in the TRP channel, which is transduced to the activation binding site, thereby precluding binding of the natural activator. Alternatively, an activity inhibitor may interfere with a component upstream or downstream of the TRP channel but which interferes with the activity of the TRP channel. This latter type of activity inhibitor is referred to as a functional antagonist. Non-limiting examples of a TRP channel inhibitor that is an activity inhibitor are gadolinium chloride, lanthanum chloride, SKF 96365 and LOE-908.

The term “vitamin” as used herein, refers to any of various organic substances essential in minute quantities to the nutrition of most animals act especially as coenzymes and precursors of coenzymes in the regulation of metabolic processes. Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

According to some embodiments, a highly lipophilic active agent complexed with HPBCD may be characterized by improved solubility in water compared to the lipophilic agent alone. According to some embodiments, a composition comprising an active-agent—inclusion complex formed with HPBCD formulated with a polymer may be characterized by slow release. According to some embodiments, a composition comprising an active-agent—inclusion complex formed with HPBCD formulated with a polymer may be characterized by controlled release. According to some embodiments, a composition comprising an active-agent—inclusion complex formed with HPBCD formulated with a polymer may be characterized by sustained release.

According to some embodiments, a composition comprising an active-agent-inclusion complex formed with HPBCD may be characterized by improved solubility compared to the active agent alone. According to some embodiments, the solubility of the compound, when present as an inclusion complex with a cyclodextrin in deionized water at 20° C., may be increased by at least about 1.5-fold, at least about 2-fold, at least about 3-fold, at least about 4-fold, at least about 5-fold, at least about 10-fold, at least about 15-fold, at least about 20-fold, at least about 30-fold, at least about 40-fold, at least about 50-fold, at least about 60-fold, at least about 70-fold, at least about 80-fold, at least about 90-fold, at least about 100-fold, at least about 200-fold, at least about 300-fold, at least about 400-fold, at least about 500-fold, at least about 1,000-fold, at least about 2,000-fold, or more over the non-complexed active agent.

According to some embodiments, a composition comprising an active-agent-inclusion complex formed with HPBCD may be characterized by reduced contact-based side effects.

According to some embodiments, the bioavailability of an active agent-inclusion complex formed with HPBCD may be improved when compared to the bioavailability, stability or both of the non-complexed active agent. According to some embodiments, the stability of an active agent-inclusion complex formed with HPBCD may be improved when compared to the stability of the non-complexed active agent. According to some embodiments, the bioavailability and stability of an active agent-inclusion complex formed with HPBCD may be improved when compared to the bioavailability, stability or both of the non-complexed active agent.

According to some embodiments, a composition comprising an active-agent-inclusion complex formed with HPBCD may be characterized by improved penetration when compared to the penetration of the non-complexed active agent. According to some embodiments, a composition comprising an active agent-inclusion complex formed with HPBCD may be characterized by improved retention when compared to the retention of the non-complexed active agent alone.

According to some embodiments, the toxicity of an active agent-inclusion complex may be reduced when compared to the toxicity of the non-complexed active agent. According to some embodiments, delivery of the composition comprising the HPBCD inclusion complex may be deliverable in a MEC to locations to which only a small amount of formulation volume is capable of being administered. This includes, without limitation, CNS delivery and ocular delivery (meaning delivery to sites adjacent to or on the eye, sites within ocular tissue, or intravitreal delivery inside the eye).

According to some embodiments, the local effective concentration of the active agent in an active agent-HPBCD inclusion complex, is increased when compared to the concentration or volume capable of being administered of the non-complexed form under the same conditions.

### Formulations

The phrase “pharmaceutically acceptable carrier” is art recognized. It is used to mean any substantially non-toxic carrier conventionally useable for administration of pharmaceuticals in which the inclusion complexes of the present invention will remain stable and bioavailable. The pharmaceutically acceptable carrier must be of sufficiently high purity and of sufficiently low toxicity to render it suitable for administration to the subject being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Exemplary carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety. According to some embodiments, the pharmaceutically acceptable carrier is sterile and pyrogen-free water. According to some embodiments, the pharmaceutically acceptable carrier is Ringer's Lactate, sometimes known as lactated Ringer's solution.

According to some embodiments, a formulation comprising: an inclusion complex comprising a) a cyclodextrin host; and b) a lipophilic guest compound, or a salt thereof, within the cavity of the cyclodextrin; and c) a carrier, are provided. According to some embodiments, the carrier is a pharmaceutically acceptable carrier. According to some embodiments, the carrier is a cosmetically acceptable carrier. According to some embodiments, the carrier may be in liquid, solid or semi-solid form. When the carrier is a liquid, it may be aqueous or an organic solvent, or a combination thereof in any amount. According to some embodiments, the carrier is selected from the group consisting of a complexing agent, a filler, a diluent, a granulating agent, a disintegrant, a lubricant, a glidant, a pH-modifier, a tonicity modifier, an adjuvant, a dye, a polymer-based film coating, and a binder. According to some embodiments, the carrier is one or more of water for injection, microcrystalline cellulose, glucose, sodium lauryl sulphate, crosscarmellose sodium, colloidal silica, talc, magnesium stearate, sodium benzoate, aluminum magnesium silicate, lactose, methanol, ethanol, propanol, and acetone. More than one carrier may be employed and combinations of carriers provided herein are intended.

According to some embodiments, the inclusion complex may comprise a lipophilic compound or a salt thereof that is partially or completely included into the cavity of a cyclodextrin molecule. According to some embodiments, the compound is fully included into the cavity of a cyclodextrin molecule. According to some embodiments, the compound is partially included into the cavity of a cyclodextrin molecule. According to some embodiments, the compound is at least 85% included into the cavity of a cyclodextrin molecule. According to some embodiments, the compound is at least 90% included into the cavity of a cyclodextrin molecule. According to some embodiments, the compound is at least 95% included into the cavity of a cyclodextrin molecule. According to some embodiments of the inclusion complex, the molar ratio of the compound to cyclodextrin is from about 10:1, about 9:1, about 8:1, about 7:1, about 6:1, about 5:1, about 4:1, about 3:1, about 2:1, about 1:1 to about 1:300; i.e., about 1:1, about 1:2, about 1:3, about 1:4, about 1:5, about 1:6, about 1:7, about 1:8, about 1:9, about 1:10, about 1:11, about 1:12, about 1:13, about 1:14: about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1: 84, about 1:85, about 1:86, about 1:87, about 1:88, about 1:89, about 1:90, about 1:91, about 1:92, about 1:93, about 1:94, about 1:95, about 1:96, about 1:97, about 1: 98, about 1:99, about 1:100.

Additives used with the inclusion complexes described herein (e.g., an inclusion complex of a compound with a cyclodextrin) include, for example, one or more excipients, one or more antioxidants, one or more stabilizers, one or more preservatives (e.g., including antimicrobial preservatives), one or more pH adjusting and/or buffering agents, one or more tonicity adjusting agents, one or more thickening agents, one or more suspending agents, one or more binding agents, one or more viscosity enhancing agents, one or more sweetening agent and the like, either alone or together with one or more additional pharmaceutical agents, provided that the additional components are pharmaceutically acceptable. According to some embodiments, the formulation may include combinations of two or more of the additional components as described herein (e.g., any of 2, 3, 4, 5, 6, 7, 8, or more additional components).

According to some embodiments, the additives include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, polyvinylpyrrolidinone, low melting waxes, ion exchange resins, and the like, as well as combinations of any two or more thereof. Other suitable pharmaceutically acceptable excipients are described in Remington's Pharmaceutical Sciences, Mack Pub. Co., New Jersey 18^{th} edition (1996), Handbook of Pharmaceutical Excipients, Pharmaceutical Press and American Pharmacists Association, 5^{th} edition (2006), and Remington: The Science and Practice of Pharmacy, Lippincott Williams & Wilkins, Philadelphia, 20^{th} edition (2003) and 21^{st} edition (2005).

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Some examples of suitable carriers, excipients, and diluents include lactose, dextrose, sucrose, sorbitol, mannitol, starches, gum acacia, calcium phosphate alginates, calcium silicate, microcrystalline cellulose, polyvinylpyrrolidone, cellulose, tragacanth, gelatin, syrup, methyl cellulose, methyl- and propylhydroxybenzoates, talc, magnesium stearate, water, and mineral oil. The formulations can additionally include lubricating agents, wetting agents, emulsifying and suspending agents, preserving agents, sweetening agents or flavoring agents. The compositions may be formulated so as to provide quick, sustained, or delayed release of the active ingredient after administration to the patient by employing procedures well known in the art.

Specific modes of administration will depend on the indication. The selection of the specific route of administration and the dose regimen is to be adjusted or titrated by the clinician according to methods known to the clinician in order to obtain the optimal clinical response. The amount of active agent to be administered is that amount sufficient to provide the intended benefit of treatment. The dosage to be administered will depend on the characteristics of the subject being treated, e.g., the particular mammal or human treated, age, weight, health, types of concurrent treatment, if any, and frequency of treatments, and can be easily determined by one of skill in the art (e.g., by the clinician).

Pharmaceutical formulations containing the active agents of the described invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels, jellies, and foams; and parenteral dosage forms which include, but are not limited to, solutions, suspensions, emulsions, and dry powder; comprising an effective amount of a polymer or copolymer of the described invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The pharmaceutical compositions of the described invention can be formulated for parenteral administration, for example, by injection, such as by bolus injection or continuous infusion. The pharmaceutical compositions can be administered by continuous infusion subcutaneously over a predetermined period of time. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents.

For oral administration, the pharmaceutical compositions can be formulated readily by combining the active agent(s) with pharmaceutically acceptable carriers well known in the art. Such carriers enable the actives of the disclosure to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, alter adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include, but are not limited to, push-fit capsules made of gelatin, as well as soft, scaled capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the described invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

In addition to the formulations described previously, the compositions of the described invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising any one or plurality of the active agents disclosed herein also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

For parenteral administration, a pharmaceutical composition can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by commonly used techniques.

The inclusion complexes may also be formulated for topical administration, especially when the target of treatment includes areas or organs readily accessible by topical application, including diseases of the eye, the skin, the lung, or the lower intestinal tract. Suitable topical formulations are readily prepared for each of these areas or organs. Topical application for the lower intestinal tract can be effected in a rectal suppository formulation or in a suitable enema formulation. Topically-applied transdermal patches may also be used.

The described invention relates to all routes of administration including topical, intramuscular, subcutaneous, sublingual, intravenous, intraperitoneal, intranasal, intratracheal, intradermal, intramucosal, intracavernous, intrarectal, into a sinus, gastrointestinal, intraductal, intrathecal, intraventricular, intrapulmonary, into an abscess, intraarticular, subpericardial, into an axilla, into the pleural space, intradermal, intrabuccal, transmucosal, transdermal, via inhalation, via nebulizer, and via subcutaneous injection. Alternatively, the pharmaceutical composition may be introduced by various means into cells that are removed from the individual. Such means include, for example, microprojectile bombardment, via liposomes or via other nanoparticle device.

According to the foregoing embodiments, the pharmaceutical composition may be administered once, for a limited period of time or as a maintenance therapy over an extended period of time, for example until the condition is ameliorated, cured or for the life of the subject. A limited period of time may be for 1 week, 2 weeks, 3 weeks, 4 weeks and up to one year, including any period of time between such values, including endpoints. According to some embodiments, the pharmaceutical composition may be administered for about 1 day, for about 3 days, for about 1 week, for about 10 days, for about 2 weeks, for about 18 days, for about 3 weeks, or for any range between any of these values, including endpoints. According to some embodiments, the pharmaceutical composition may be administered for more than one year, for about 2 years, for about 3 years, for about 4 years, or longer.

According to some embodiments, the inclusion complexes may be administered with an additional therapeutic agent and/or an additional treatment modality. The dosing frequency of the inclusion complex and the additional pharmaceutical agent may be adjusted over the course of the treatment based on the judgment of the administering physician. When administered separately, the inclusion complex and the additional therapeutic agent can be administered at different dosing frequency or intervals. For example, the inclusion complex can be administered weekly, while the additional therapeutic agent can be administered more or less frequently. In some embodiments, sustained continuous release formulation of the inclusion complex and/or the additional therapeutic agent may be used. Various formulations and devices for achieving sustained release are known in the art. A combination of the administration configurations described herein can be used. In some embodiments, the inclusion complex can be administered daily and the additional therapeutic agent can be administered monthly. In some embodiments, the inclusion complex can be administered weekly and the additional therapeutic agent can be administered monthly.

According to the foregoing embodiments, the composition or pharmaceutical composition may be administered once daily, twice daily, three times daily, four times daily or more.

Also provided are unit dosage forms comprising the inclusion complexes and formulations described herein. These unit dosage forms can be stored in a suitable packaging in single or multiple unit dosages and may also be further sterilized and sealed.

All referenced journal articles, patents, and other publications are incorporated by reference herein in their entirety.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

### EXAMPLES

### Example 1: Physical Characterization of HPBCD Inclusion Complexes

Inclusion Complex Formation.

The required amount of dry HPBCD is weighed out at room temperature. A vacuum is established. The active, which is substantially free of solvent (either aqueous or organic), is added to the HPBCD under vacuum.

Analytical Method.

UV-Vis was used for identification and quantification of active agents and degradation products. An Agilent Cary 60 UV-Vis spectrophotometer with a double beam, Czerny-Turner monochromator, 1.5 nm fixed spectral bandwidth, full spectrum Xenon pulse lamp and wavelength range of 190-1100 nm was used for analysis. A scan rate of 4800 nm/s was employed, with samples run in triplicate. The wavelength of analysis varied per sample, and was selected specifically for each active, based on their spectra. All native actives were dissolved in 200 proof ethanol. All HPBCD complexes and native HPBCD were dissolved in deionized water.

Phase Solubility Studies.

The effects of HPBCD on the solubility of each active agent (hereinafter “active”) is studied by the phase solubility method in USP buffer pH 4. Based on its molecular weight, appropriate amounts HPBCD are added to solution. 0 to 7 mM concentration solutions of HPBCD in pH 4 are prepared and maintained at required temperature (25, 30, 35° C.). The active is added to the above prepared solution in excess amount in test tubes. Test tubes are sealed using paraffin and stored in an incubator shaker. Active concentrations in the solutions are measured using HPLC at 4 hr time intervals.

Degradation and Effect of CDs on Degradation Rate.

Actives are dissolved in appropriate amount of water depending upon concentration, and desired temperature is maintained. 1 N HCl is maintained at the same temperature. The required amount of HCl is added to the actives solution. Samples withdrawn from these solutions at predetermined time intervals are neutralized to stop further degradation and analyzed using HPLC. Degradation rates in the presence of HPBCD in solution are determined. An appropriate amount of HPBCD is added along with the active agent in water to achieve a HPBCD concentration of 1, 5, and 10 mg/ml. Studies are performed in 0.1 N (pH 1), 0.05 N (pH 1.3), 0.025 N (pH 0.6) HCl concentrations at three different temperatures (25, 30, 35° C.).

Content Uniformity.

Content uniformity of the active in prepared complexes is investigated by active recovery studies where known quantities of the active and active-HPBCD complex are dissolved in a 10 ml mobile phase to get a clear solution. The solution is further diluted with mobile phase and buffer before analyzing using HPLC.

Thermal Analysis.

Calorimetric studies are conducted using a Modulated Differential Scanning calorimetry instrument (MDSC). Accurately weighed samples are sealed in Tzero aluminum pans. Empty sealed Tzero aluminum pans are used as a reference. Both pans are heated at a rate of 10° C./min with +/−1.59 modulations every 60 mins from 40° C. to 250° C. under nitrogen gas flow of 20 ml/min. Thermal analysis of pure active, excipients, formulations and physical mixtures are performed. Data analysis is performed using Universal Analysis software to measure melting point enthalpy of melting.

X-Ray Diffraction.

X-ray diffraction (XRD) patterns are studied to verify whether active-CD complexation caused any structural changes in the compound. A scanning X-ray diffractometer is used in this study. X-ray diffraction patterns are obtained for the active, HPBCD, drug—HPBCD complex, and drug-HPBCD physical mixture. Radiation used is generated by a copper Kα filter, with wavelength 1.54 A° at 35 kV and 30 mA. A glass slide is covered with the sample to be analyzed and scanned over a range from 5° to 40° 20 degrees, using a scan rate of 1 degree per min and a step scan of 0.02.

Infrared Spectroscopy.

MAGNA-IR 760 Spectrophotometer (Thermo Scientific, USA) is used to obtain Infrared (IR) spectra for all sample powders. Powdered potassium bromide (KBr) of IR grade stored in desiccators is used as background material. Minute quantity of each sample is triturated with pure KBr using a mortar and pestle to form a uniform mixture, then compressed to form a semi-transparent film. Each film is scanned (64 scans) in the region of 400 to 4000 cm⁻¹ in transmittance mode. Essential FTIR software is used to detect any shift or disappearance of absorption peak in spectra due to formation of any bond between the active and CD.

Scanning Electron Microscopy.

Scanning electron microscopy (SEM) is conducted to observe surface morphology and texture of pure materials and binary blends. The SEM photographs are taken using JEOL Scanning electron microscope, model 5900 LV. The samples are mounted on double sided carbon tape 31 for SEM imaging. Low Vacuum (LV) mode is used to prevent the samples from charging. The analyses are conducted using 1000× magnification.

Particle Size.

The terms “D value” or “mass division diameter” as used herein, refer to the diameter which, when all particles in a sample are arranged in order of ascending mass, divides the sample's mass into specified percentages. The percentage mass below the diameter of interest is the number expressed after the “D”. For example, the D10 diameter is the diameter at which 10% of a sample's mass is comprised of smaller particles, and the D50 is the diameter at which 50% of a sample's mass is comprised of smaller particles. The D50 is also known as the “mass median diameter” as it divides the sample equally by mass. The D90 diameter is the diameter at which 90% of a sample's mass is comprised of smaller particles. While D-values are based on a division of the mass of a sample by diameter, the actual mass of the particles or the sample does not need to be known. A relative mass is sufficient as D-values are concerned only with a ratio of masses. This allows optical measurement systems to be used without any need for sample weighing. From the diameter values obtained for each particle a relative mass can be assigned according to the following relationship:

Mass of a sphere=ττ/6d³p

Assuming that p is constant for all particles and cancelling all constants from the equation: Relative mass=d³, i.e., each particle's diameter is therefore cubed to give its relative mass. These values can be summed to calculate the total relative mass of the sample measured. The values may then be arranged in ascending order and added iteratively until the total reaches 10%, 50% or 90% of the total relative mass of the sample. The corresponding D value for each of these is the diameter of the last particle added to reach the required mass percentage.

Dissolution Studies.

The term “dissolution rate” as used herein refers to the amount of a drug that dissolves per unit time. The term “inherent dissolution rate” is the dissolution rate of a pure API under constant conditions of surface area, rotation speed, pH and ionic strength of the dissolution medium. Inherent dissolution rate is applicable to the determination of thermodynamic parameters associated with different crystalline phases and their solution-mediated phase transformations, investigation of the mass transfer phenomena during the dissolution process, determination of pH-dissolution rate profiles, and the evaluation of the impact of different pH values and the presence of surfactants on the solubilization of poorly soluble compounds.

Active (280 mg) and various active-HPBCD mixtures (equivalent to 280 mg of drug) are analyzed using USP apparatus-II for in-vitro dissolution studies. The dissolution studies are carried out at 37.2° C. with rotation speed of 75 RPM in 250 ml volumes for pH 1, 2, and 4 and 900 ml for pH 5.5 buffers to mimic gastrointestinal fluid conditions. 5 mL aliquots are withdrawn from the dissolution medium, and an equal amount of fresh media is replaced at time=5, 10, 15, 20, 25, 30, 45, 60, 90, 120, and 180 min. Withdrawn samples are filtered using 0.45 μm pore size filters and further diluted with buffer and mobile phase to prevent active degradation during HPLC analysis.

The terms “drug load (%)” and “drug loading capacity” are used interchangeably to refer to a ratio of the weight of a drug/active agent in the HPBCD inclusion complex relative to the total weight of the inclusion complex, expressed as a percentage. It reflects the drug content of the inclusion complex.

### Example 2: Characterization of Hydroxypropyl β-Cyclodextrin (HPBCD) Inclusion Complexes

Hydroxypropyl β-cyclodextrin (HPBCD, molecular eight 1375.37 g/mol) was used as a complexing agent to enhance the delivery and permeation of a number of active compounds into and across the skin. HPBCD is a partially substituted poly(hydroxypropyl) ether of β-cyclodextrin, and is an approved excipient with monographs in both the US Pharmacopoeia 28/National Formulary 23 and the European Pharmacopoeia.

An inclusion complex of each active with HBPCD at an active:HBPCD mole ratio of 1:1 (e.g., niacinamide, CBD, and benzocaine); 1:2 (e.g., minoxidil), or 1:3 (e.g., tamanu oil, TC, pycnogenol) was prepared. The required amount of dry HPBCD was weighed out at room temperature, and a vacuum established. Each active which is substantially free of solvent (organic or aqueous) was added to the HPBCD under vacuum. There was no seepage or separation.

Analytical Method.

UV-Vis was used for identification and quantification of active agents and degradation products.

As shown in FIG. 3A, Benzocaine displays peak maximums at 272 nm and 296 nm. The HPBCD benzocaine complex exhibits peak maximums at 260 nm, 290 nm, and 310 nm. HPBCD has a small broad peak at 241 nm. This shows the cyclodextrin molecule does not interfere in the active region of benzocaine, thus UV can be used for analysis of the complex.

As shown in FIG. 3B, CBD displays peak maximums at 221 nm, 233 nm, 239 nm and 278 nm. The HPBCD CBD complex exhibits peak maximums at 221 nm, 227 nm, 233 nm and 278 nm. HPBCD has a small broad peak at 241 nm. This shows the cyclodextrin molecule does not interfere in the prominent active region of CBD, thus UV can be used for analysis of the complex.

As shown in FIG. 3C, Minoxidil displays peak maximums at 230 nm, 250 nm, 260 nm, 280 nm and 290 nm. The HPBCD minoxidil complex exhibits peak maximums at 255 nm and 280 nm. HPBCD has a small broad peak at 241 nm. This shows the cyclodextrin molecule does not interfere in the active region of minoxidil, thus UV can be used for analysis of the complex.

As shown in FIG. 3D, Niacinamide displays peak maximums at 235 nm and 255 nm. The HPBCD niacinamide complex exhibits peak maximums at 240 nm, 265 nm, and 295 nm. HPBCD has a small broad peak at 241 nm. This shows the cyclodextrin molecule does not interfere in the prominent active region of niacinamide, thus UV can be used for analysis of the complex.

As shown in FIG. 3E, Pycnogenol displays peak maximums at 230 nm, 280 nm and 310 nm. The HPBCD pycnogenol complex exhibits peak maximums at 225 nm, 240 nm, 275 nm and 305 nm. HPBCD has a small broad peak at 241 nm. This shows the cyclodextrin molecule does not interfere in the prominent active region of pycnogenol, thus UV can be used for analysis of the complex.

As shown in FIG. 3F, Tamanu oil displays peak maximums at 215 nm, 269 nm and 296 nm. The HPBCD tamanu oil complex exhibits peak maximums at 206 nm, 212 nm, 218 nm, 262 nm and 366 nm. HPBCD has a small broad peak at 241 nm. This shows the cyclodextrin molecule does not interfere in the active region of tamanu oil, thus UV can be used for analysis of the complex.

As shown in FIG. 3G, Tetrahydrocurcumin displays peak maximums at 209 nm, 218 nm and 278 nm. The HPBCD tetrahydrocurcumin complex exhibits peak maximums at 225 nm and 280 nm. HPBCD has a small broad peak at 241 nm. This shows the cyclodextrin molecule does not interfere in the active region of tetrahydrocurcumin, thus UV can be used for analysis of the complex.

Differential Scanning Calorimetry.

Differential scanning calorimetry was used to determine the amount of the active that remained noncomplexed. Differential scanning calorimetry (DSC) is a thermoanalytical technique useful in detecting phase transitions in solid samples by measuring the amount of heat absorbed or released during such transitions. DSC provided melting point data pertinent to characterizing the inclusion complex formed between the Actives and HPBCD.

DSC analysis was performed using a TA Trios DSC instrument. Samples tested were HPBCD, the Active, and the active-HPBCD inclusion complexes. Each weighed sample for analysis ranged from 2.00 mg to 4.00 mg.

Cyclodextrin (CD) is a large, carbohydrate molecule. Due to the lack of a crystalline nature of the CD, the DSC spectra shows a characteristic broad peak around 100° C., due to water loss. Moisture from the atmosphere readily bonds to the outer portion of CD. All the complexes used in the Skin Permeability Study utilized the hydroxypropyl beta analog of cyclodextrin (abbreviated as HP-B-CD).

If the guest molecule has a crystalline nature, there will be a sharp melting peak in its DSC spectrum. If the guest is fully incorporated into the cavity of the host, the crystallinity diminishes, and the resulting spectrum should look very similar to the spectrum for cyclodextrin. If the guest is partially included within the host, there will be a small melting peak corresponding to the portion of the guest molecule that is hanging outside the CD cavity.

The central cavity size of HPBCD is about 6.0-6.5 Daltons. For some of the larger molecules, such as CBD or Tetrahydrocurcumin (TC), there is a portion of the molecule that sticks out of the cyclodextrin cavity after complexation.

Each inclusion complex is soluble in water.

The results are described below and shown in FIGS. 4-10.

Niacinamide (molecular weight 122.127 g/mol): FIG. 4 shows overlaid DSC curve for niacinamide (green), with a single melting peak at about 135° C.; HPBCD (red) with a broad melting curve that peaks at about 100° C., and HPBCD niacinamide inclusion complex (blue), with no niacinamide melting peak present, but a broad melting curve that peaks at around 100° C. Since niacinamide is a relatively small molecule, it fully fits within the cavity of the CD host. Thus the spectrum of the complex looks very similar to the spectrum of native HP-B-CD. These overlaid spectra show full inclusion within cyclodextrin.

Tamanu oil (molecular weight 873.4 g/mol): FIG. 5 shows overlaid DSC curves for Tamanu oil, which has no discernable melting peak (red), HPBCD (green) with a melting peak at about 106° C.; and HPBCD tamanu inclusion complex (blue), with a melting peak at about 112.5° C. As an oil, tamanu oil is lacking a definitive crystalline nature. Therefore its spectrum does not yield a sharp melting peak, although there are some characteristic phenomena occurring in the 210-250° C. range. These characteristic peaks disappeared in the spectrum of the tamanu oil-HPBCD complex; thus full inclusion of the oil was achieved.

Cannabidiol (CBD) (molecular weight 314.464 g/mol): FIG. 6 shows overlaid DSC curves for crystalline CBD (green) with a sharp melting peak at about 65° C.; a melting curve for HPBCD with a minimum of about 106° C., and HPBCD-CBD inclusion complex (blue), with a broad melting peak at about 110° C. Due to the large size of the CBD molecule, only a portion of the CBD fits inside the HP-B-CD cavity. In the spectrum of the complex, a smaller melting peak is observed, corresponding to the portion of BBD hanging outside the cavity, which is shifted to around 60° C. due to steric hindrance.

Tetrahydrocurcumin (molecular weight, 372.417 g/mol): FIG. 7 shows overlaid DSC curves for tetrahydrocurcumin (green) with a single melting peak at about 106° C.; HPBCD with a broad melting curve (red) with a minimum at about 104° C.; and HPBCD tetrahydrocurcumin inclusion complex (blue), with a broad melting peak at about 110° C. There is a small melting peak around 88° C., which corresponds to the portion of the tetrahydrocurcumin that is hanging outside the cyclodextrin cavity. It is shifted from the overall tetrahydrocurcumin melting peak around 104° C., since it is only a part of the molecule, and because the complexation with cyclodextrin diminishes the crystallinity of, and imparts steric hindrance on, the molecule.

Benzocaine (molecular weight 165.19 g/mol). FIG. 8 shows overlaid DSC curves for benzocaine (green), which displays a very sharp melting peak around 90° C., as well as a smaller broader peak at around 180° C. before full decomposition at 230° C., HPBCD with a broad melting curve (blue), and HPBCD benzocaine inclusion complex (red). After complexation with cyclodextrin, the benzocaine melting peaks disappear, indicating full inclusion within the cyclodextrin cavity. This also shows the prevention of decomposition of benzocaine at 230° C., indicating that the stability of the molecule is enhanced by cyclodextrin complexation.

Minoxidil (molecular weight 209.251 g/mol). FIG. 9 shows overlaid DSC curves for minoxidil (red), which displays a very sharp melting peak around 180 C, HPBCD with a broad melting curve (green), and HPBCD minoxidil inclusion complex (blue). After complexation with cyclodextrin, the minoxidil melting peak disappears, indicating full inclusion within the cyclodextrin cavity.

Pycnogenol Pinus pinaster, bark extract (molecular weight 1155.03 g/mol). As an extract, Pycnogenol is made up of several molecules. It consists of 65-75% proanthocyanidins, and contains phenolic acids. The structural formula of the dimeric type proanthocyanidins is C₃₀H₂₆O₁₂ with molecular weight 578.52 g/mol. The structural formula of Procyanadin A1 and A2 is C₃₀H₂₄O₁₂ with molecular weight 576.51 g/mol.

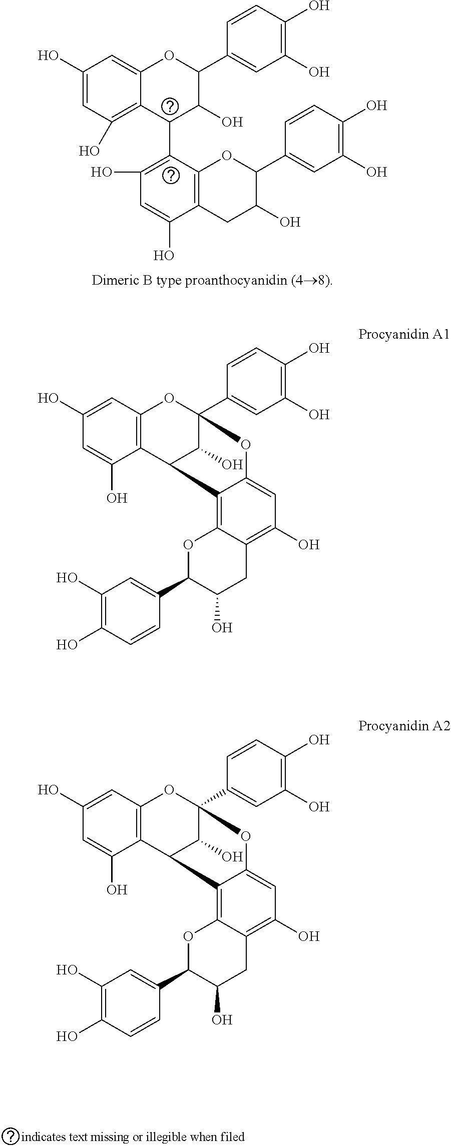 [SMILES: Oc1cc(O)c2c(c1)OC(c1ccc(O)c(O)c1)C(O)C2c1c(O)cc(O)c2c1OC(c1ccc(O)c(O)c1)C(O)C2 ; Oc1cc(O)c2c(c1)O[C@@]1(c3ccc(O)c(O)c3)Oc3cc(O)c4c(c3[C@@H]2[C@H]1O)O[C@H](c1ccc(O)c(O)c1)[C@@H](O)C4 ; Oc1cc(O)c2c(c1)O[C@@]1(c3ccc(O)c(O)c3)Oc3cc(O)c4c(c3[C@@H]2[C@H]1O)O[C@H](c1ccc(O)c(O)c1)[C@H](O)C4]

Assuming that the weight is a combination of type B and type A, the estimated molecular weight of pycnogenol is 1155.03 g/mol (578.52+576.51).

FIG. 10 shows overlaid DSC curves for pycnogenol (green), HPBCD with a broad melting curve (blue), and HPBCD-pycogenol inclusion complex (red). Pycnogenol, being a plant extract and thus made up of several different molecules, does not have a definitive crystalline nature; thus there is no sharp melting peak in the spectrum. However, it does display a very broad curve with minimums at around 100° C. and 112° C., with decomposition occurring at 210° C. After complexation with cyclodextrin, there is a small, very broad hump with a median value around 195° C., due to the portion of the pycnogenol hanging outside the cyclodextrin cavity. Complexation also increases the stability of pycnogenol, as the decomposition does not start occurring until around 240° C.

Table 2 below shows the pH of the HPBCD complexes shown dissolved in deionized water solutions.

TABLE 2

| | pH | | | | | | |
|---|---|---|---|---|---|---|---|
| Inclusion | 1% | 5% | 10% | 15% | 20% | 25% | 30% |
| complex | soln, | soln | soln | soln | soln | soln | soln |
| HPBCD-tamanu | 7.24 | 6.46 | 6.12 | 5.93 | 5.72 | 5.56 | 5.54 |
| Oil | | | | | | | |
| HPBCD-CBD | 7.14 | 7.29 | 7.30 | 7.51 | 7.40 | 7.44 | 7.43 |
| HPBCD- | 6.80 | 7.03 | 7.27 | 7.35 | 7.37 | 7.37 | 7.37 |
| Niacinamide | | | | | | | |
| HPBCD- | 7.46 | 7.34 | 7.27 | 7.23 | 7.18 | 7.18 | 7.18 |
| Tetrahydro- | | | | | | | |
| curcumin | | | | | | | |
| HHP-BCD- | 6.74 | 6.91 | 7.01 | 7.01 | 7.01 | 7.01 | 7.02 |
| Benzocaine | | | | | | | |
| HPBCD- | 7.20 | 7.34 | 7.39 | 7.41 | 7.42 | 7.49 | 7.51 |
| Minoxidil | | | | | | | |
| HPBCD- | 4.5 | 3.7 | 3.5 | 3.40 | 3.34 | 3.30 | 3.27 |
| Pycnogenol | | | | | | | |

Stability Studies.

The effects of HPBCD on the shelf life stability of each active agent is studied at pre-determined temperatures for 11 weeks. Real time stability is observed at −17° C., 5° C. and 25° C., and accelerated stability is observed at 40° C. For accelerated stability, one day at 40° C. is equivalent to one week, thus the data represents 77 weeks. The HPBCD complexes and active agents are placed in a 5-dram glass vial at a weight of 1 gram. The vials are then placed in a temperature-controlled oven or refrigerator/freezer. The compounds are checked daily and any visible changes are noted.

TABLE 3
Stability of Complexes at 25° C.

| | | | HPBCD | HPBCD- | | | |
|---|---|---|---|---|---|---|---|
| | HPBCD- | HPBCD- | tamanu | tetrahydro | HPBCD | HPBCD | HPBCD- |
| At 25° C. | niacinamide | CBD | oil | curcumin | benzocaine | minoxcidil | pycogenol |
| Week 1 | 0 | 0 | 0 | 0 | 0 | 0 | 0 |
| Week 2 | 0 | 0 | 0 | 0 | 0 | 0 | 0 |
| Week 3 | 0 | 0 | 0 | 0 | 0 | 0 | 0 |
| Week 4 | 0 | 0 | 0 | 0 | 0 | 0 | 0 |
| Week 5 | 0 | 0 | 0 | 0 | 0 | 0 | 0 |
| Week 6 | 0 | 0 | 0 | 0 | 0 | 0 | 0 |
| Week 7 | 0 | 0 | 0 | 0 | 0 | 0 | 0 |
| Week 8 | 0 | 0 | 0 | 0 | 0 | 0 | 0 |
| Week 9 | 0 | 0 | 0 | 0 | 0 | 0 | 0 |
| Week 10 | 0 | 0 | 0 | 0 | 0 | 0 | 0 |
| Week 11 | 0 | 0 | 0 | 0 | 0 | 0 | 0 |

0 = no change;
c = clumped

TABLE 4
Stability of Actives at 25° C.

| | | | | Tamanu | Tetrahydro | | | |
|---|---|---|---|---|---|---|---|---|
| At 25° C. | HPBCD | Niacinamide | CBD | Oil | curcumin | Benzocaine | Minoxidil | Pycogenol |
| Week 1 | 0 | 0 | 0 | 0 | 0 | 0 | 0 | 0 |
| Week 2 | 0 | 0 | 0 | 0 | 0 | 0 | 0 | 0 |
| Week 3 | 0 | 0 | 0 | 0 | 0 | 0 | 0 | 0 |
| Week 4 | 0 | 0 | c | 0 | 0 | 0 | 0 | 0 |
| Week 5 | 0 | 0 | c | 0 | 0 | 0 | 0 | 0 |
| Week 6 | 0 | 0 | c | 0 | 0 | 0 | 0 | 0 |
| Week 7 | 0 | 0 | c | 0 | 0 | 0 | 0 | 0 |
| Week 8 | 0 | 0 | c | 0 | 0 | 0 | 0 | 0 |
| Week 9 | 0 | 0 | c | 0 | 0 | 0 | 0 | 0 |
| Week 10 | 0 | 0 | c | 0 | 0 | 0 | 0 | 0 |
| Week 11 | 0 | 0 | c | 0 | 0 | 0 | 0 | 0 |

0 = no change;
c = clumped

TABLE 5
Stability of Complexes at 40° C.

| | | | HPBCD | HPBCD- | | | |
|---|---|---|---|---|---|---|---|
| | HPBCD- | HPBCD- | tamanu | tetrahydro | HPBCD | HPBCD | HPBCD- |
| At 40° C. | niacinamide | CBD | oil | curcumin | benzocaine | minoxcidil | pycogenol |
| Week 1 | 0 | 0 | 0 | 0 | 0 | 0 | 0 |
| Week 2 | 0 | 0 | 0 | 0 | 0 | 0 | 0 |
| Week 3 | 0 | 0 | 0 | 0 | 0 | 0 | 0 |
| Week 4 | 0 | 0 | 0 | 0 | 0 | 0 | 0 |
| Week 5 | 0 | 0 | 0 | 0 | 0 | 0 | 0 |
| Week 6 | 0 | sc | 0 | 0 | 0 | 0 | 0 |
| Week 7 | 0 | sc | 0 | 0 | 0 | 0 | 0 |
| Week 8 | 0 | sc | 0 | 0 | 0 | 0 | 0 |
| Week 9 | 0 | sc | 0 | 0 | 0 | 0 | 0 |
| Week 10 | 0 | sc | sc | 0 | 0 | 0 | 0 |
| Week 11 | 0 | sc | sc | 0 | 0 | 0 | 0 |

0 = no change;
sc = slightly clumped;
c = clumped

TABLE 6
Stability of Actives at 40° C.

| | | | | Tamanu | Tetrahydro | | | |
|---|---|---|---|---|---|---|---|---|
| At 40° C. | HPBCD | Niacinamide | CBD | Oil | curcumin | Benzocaine | Minoxidil | Pycogenol |
| Week 1 | 0 | 0 | c | 0 | 0 | 0 | 0 | 0 |
| Week 2 | 0 | sc | c | 0 | 0 | 0 | 0 | 0 |
| Week 3 | 0 | sc | c | 0 | 0 | 0 | 0 | 0 |
| Week 4 | 0 | sc | c | 0 | 0 | 0 | 0 | 0 |
| Week 5 | 0 | sc | c | 0 | 0 | 0 | 0 | 0 |
| Week 6 | 0 | sc | c | 0 | 0 | 0 | 0 | 0 |
| Week 7 | 0 | sc | c | 0 | 0 | 0 | 0 | 0 |
| Week 8 | 0 | sc | c | 0 | 0 | 0 | 0 | 0 |
| Week 9 | 0 | sc | c | 0 | 0 | 0 | 0 | 0 |
| Week 10 | 0 | sc | c | 0 | 0 | 0 | 0 | 0 |
| Week 11 | 0 | sc | c | 0 | 0 | 0 | 0 | 0 |

0 = no change;
sc = slightly clumped;
c = clumped

TABLE 7
Stability of Complexes at 5° C.

| | | | HPBCD | HPBCD- | | | |
|---|---|---|---|---|---|---|---|
| | HPBCD- | HPBCD- | tamanu | tetrahydro | HPBCD | HPBCD | HPBCD- |
| At 5° C. | niacinamide | CBD | oil | curcumin | benzocaine | minoxcidil | pycogenol |
| Week 1 | 0 | 0 | 0 | 0 | 0 | 0 | 0 |
| Week 2 | 0 | 0 | 0 | 0 | 0 | 0 | 0 |
| Week 3 | 0 | 0 | 0 | 0 | 0 | 0 | 0 |
| Week 4 | 0 | 0 | 0 | 0 | 0 | 0 | 0 |
| Week 5 | 0 | 0 | 0 | 0 | 0 | 0 | 0 |
| Week 6 | 0 | 0 | 0 | 0 | 0 | 0 | 0 |
| Week 7 | 0 | 0 | 0 | 0 | 0 | 0 | 0 |
| Week 8 | 0 | 0 | 0 | 0 | 0 | 0 | 0 |
| Week 9 | 0 | 0 | 0 | 0 | 0 | 0 | 0 |
| Week 10 | 0 | 0 | 0 | 0 | 0 | 0 | 0 |
| Week 11 | 0 | 0 | 0 | 0 | 0 | 0 | 0 |

0 = no change;
c = clumped;
f = frozen;
pf = partially frozen

TABLE 8
Stability of Actives at 5° C.

| | | | | Tamanu | Tetrahydro | | | |
|---|---|---|---|---|---|---|---|---|
| At 5° C. | HPBCD | Niacinamide | CBD | Oil | curcumin | Benzocaine | Minoxidil | Pycogenol |
| Week 1 | 0 | 0 | c | f | 0 | 0 | 0 | 0 |
| Week 2 | 0 | pf | c | f | 0 | 0 | 0 | 0 |
| Week 3 | 0 | pf | c | f | 0 | 0 | 0 | 0 |
| Week 4 | 0 | pf | c | f | 0 | 0 | 0 | 0 |
| Week 5 | 0 | pf | c | f | 0 | 0 | 0 | 0 |
| Week 6 | 0 | pf | c | f | 0 | 0 | 0 | 0 |
| Week 7 | 0 | pf | c | f | 0 | 0 | 0 | 0 |
| Week 8 | 0 | pf | c | f | 0 | 0 | 0 | 0 |
| Week 9 | 0 | pf | c | f | 0 | 0 | 0 | 0 |
| Week 10 | 0 | pf | c | f | 0 | 0 | 0 | 0 |
| Week 11 | 0 | pf | c | f | 0 | 0 | 0 | 0 |

0 = no change;
c = clumped;
f = frozen;
pf = partially frozen

TABLE 9
Stability of Complexes at −17° C.

| | | | HPBCD | HPBCD- | | | |
|---|---|---|---|---|---|---|---|
| | HPBCD- | HPBCD- | tamanu | tetrahydro | HPBCD | HPBCD | HPBCD- |
| At −17° C. | niacinamide | CBD | oil | curcumin | benzocaine | minoxcidil | pycogenol |
| Week 1 | 0 | 0 | 0 | 0 | 0 | 0 | 0 |
| Week 2 | 0 | 0 | Pf | 0 | 0 | 0 | 0 |
| Week 3 | 0 | pf | pf | 0 | 0 | 0 | 0 |
| Week 4 | 0 | pf | pf | 0 | 0 | 0 | 0 |
| Week 5 | 0 | pf | pf | 0 | 0 | 0 | 0 |
| Week 6 | 0 | pf | pf | 0 | 0 | 0 | 0 |
| Week 7 | pf | pf | pf | 0 | 0 | 0 | 0 |
| Week 8 | pf | pf | pf | 0 | 0 | 0 | 0 |
| Week 9 | pf | pf | pf | 0 | 0 | 0 | 0 |
| Week 10 | pf | pf | pf | 0 | 0 | 0 | 0 |
| Week 11 | pf | pf | pf | 0 | 0 | 0 | 0 |

0 = no change;
c = clumped;
f = frozen;
pf = partially frozen

TABLE 10
Stability of Actives at −17 C.

| | | | | Tamanu | Tetrahydro | | | |
|---|---|---|---|---|---|---|---|---|
| At 25° C. | HPBCD | Niacinamide | CBD | Oil | curcumin | Benzocaine | Minoxidil | Pycogenol |
| Week 1 | 0 | 0 | pf | f | 0 | 0 | 0 | |
| Week 2 | 0 | pf | pf | f | 0 | 0 | 0 | |
| Week 3 | 0 | pf | pf | f | 0 | 0 | 0 | |
| Week 4 | 0 | pf | pf | f | 0 | 0 | 0 | |
| Week 5 | 0 | pf | pf | f | 0 | 0 | 0 | |
| Week 6 | 0 | pf | pf | f | 0 | 0 | 0 | |
| Week 7 | 0 | pf | pf | f | 0 | 0 | 0 | |
| Week 8 | 0 | pf | pf | f | 0 | 0 | 0 | |
| Week 9 | 0 | pf | pf | f | 0 | 0 | 0 | |
| Week 10 | 0 | pf | pf | f | 0 | 0 | 0 | |
| Week 11 | 0 | pf | pf | f | 0 | 0 | 0 | |

0 = no change;
f = frozen;
c = clumped;
pf = partially frozen

Dissolution Studies.

The results of dissolution studies are shown in FIG. 11-17.

A dissolution study of HPBCD benzocaine complex was performed using the compound as a dry granulation. A slightly higher percentage of the active was dissolved at higher pH value. The dissolution profile (FIG. 11A) displays a burst like, zero-order release. A zero-order release implies the active release is independent of the initial drug concentration. Typically, zero-order release is achieved from non-disintegrating dosage forms such as topical or transdermal delivery systems, as well as oral controlled release systems for drugs with low solubility. A concentration curve of the complex (FIG. 11B) was created, and the resulting equation was utilized to calculate the percentage of drug released. The wavelength for analysis of HPBCD benzocaine complex was 290 nm.

A dissolution study of HPBCD CBD complex was performed using the compound as a dry granulation. A slightly higher percentage of the active was dissolved at higher pH value. The dissolution profile (FIG. 12A) adopts the characteristic shape of a sustained release profile. Sustained release implies the drug is released over a longer period of time, with the percentage decreasing slightly over time. This type of profile can also be considered as zero-order. Typically, zero-order release is achieved from non-disintegrating dosage forms such as topical or transdermal delivery systems, as well as oral controlled release systems for drugs with low solubility. CBD is completely insoluble in water, and this shows that complexing with cyclodextrin allows a percentage of the active to be dissolved in an aqueous system. A concentration curve of the complex (FIG. 12B) was created, and the resulting equation was utilized to calculate the percentage of drug released. The wavelength for analysis of HPBCD CBD complex was 233 nm.

A dissolution study of HPBCD minoxidil complex was performed using the compound as a dry granulation. A substantially higher percentage of the active was dissolved at lower pH value. The dissolution profile (FIG. 13A) displays a burst like, zero-order release. A zero-order release implies the active release is independent of the initial drug concentration. Typically, zero-order release is achieved from non-disintegrating dosage forms such as topical or transdermal delivery systems, as well as oral controlled release systems for drugs with low solubility. A concentration curve of the complex (FIG. 13B) was created, and the resulting equation was utilized to calculate the percentage of drug released. The wavelength for analysis of HPBCD minoxidil complex was 280 nm.

A dissolution study of HPBCD niacinamide complex was performed using the compound as a dry granulation. A higher percentage of the active was dissolved at lower pH value. The dissolution profile (FIG. 14A) displays a burst like, zero-order release. A zero-order release implies the active release is independent of the initial drug concentration. Typically, zero-order release is achieved from non-disintegrating dosage forms such as topical or transdermal delivery systems, as well as oral controlled release systems for drugs with low solubility. A concentration curve of the complex (FIG. 14B) was created, and the resulting equation was utilized to calculate the percentage of drug released. The wavelength for analysis of HPBCD niacinamide complex was 265 nm.

A dissolution study of HPBCD pycnogenol complex was performed using the compound as a dry granulation. The percentage of the active dissolved was virtually the same at lower and higher pH value. The dissolution profile (FIG. 15A) displays a burst like, zero-order release. A zero-order release indicates the active release is independent of the initial drug concentration. Typically, zero-order release is achieved from non-disintegrating dosage forms such as topical or transdermal delivery systems, as well as oral controlled release systems for drugs with low solubility. A concentration curve of the complex (FIG. 15B) was created, and the resulting equation was utilized to calculate the percentage of drug released. The wavelength for analysis of HPBCD pycnogenol complex was 225 nm.

A dissolution study of HPBCD tamanu oil complex was performed using the compound as a dry granulation. A higher percentage of the active was dissolved at higher pH value. The dissolution profile (FIG. 16A) adopts the characteristic shape of a sustained release profile. Sustained release implies the drug is released over a longer period of time, with the percentage decreasing slightly over time. This type of profile can also be considered as zero-order. Typically, zero-order release is achieved from non-disintegrating dosage forms such as topical or transdermal delivery systems, as well as oral controlled release systems for drugs with low solubility. Tamanu oil is completely insoluble in water, and this shows that complexing with cyclodextrin allows a percentage of the active to be dissolved in an aqueous system. A concentration curve of the complex (FIG. 16B) was created, and the resulting equation was utilized to calculate the percentage of drug released. The wavelength for analysis of HPBCD tamanu oil complex was 212 nm.

A dissolution study of HPBCD tetrahydrocurcumin complex was performed using the compound as a dry granulation. The percentage of the active dissolved was similar at lower and higher pH value. Interestingly, at lower pH the percentage of active dissolved decreases somewhat over time, resembling a sustained release profile. The dissolution profile (FIG. 17A) displays a burst like, zero-order release. A zero-order release indicates the active release is independent of the initial drug concentration. Typically, zero-order release is achieved from non-disintegrating dosage forms such as topical or transdermal delivery systems, as well as oral controlled release systems for drugs with low solubility. A concentration curve of the complex (FIG. 17B) was created, and the resulting equation was utilized to calculate the percentage of drug released. The wavelength for analysis of HPBCD tetrahydrocurcumin complex was 225 nm.

Drug Load (%)

Drug loading capacity of the HPBCD inclusion complexes is shown in Table 11.

TABLE 11
Drug loading capacity of HPBCD inclusion complexes.

| Complex | Drug Load (%) |
|---|---|
| HP-B-CD Niacinamide | 8.88 |
| HP-B-CD Tetrahydrocurcumin | 9.03 |
| HP-B-CD Tamanu Oil | 21.17 |
| HP-B-CD CBD | 22.86 |
| HP-B-CD Minoxidil | 7.61 |
| HP-B-CD Benzocaine | 12.01 |
| HP-B-CD Pycnogenol | 27.99 |

### Example 3. Phase Solubility Studies

FIG. 18 is an A_{L} type phase solubility diagram showing the phase solubility diagram for components S and L. A linear increase in the solubility of S is classified as AL type by Higuchi and Connors [Phase-solubility techniques, Adv. Anal. Chem. Instr. 4, 117-122, (1965)] and demonstrates that the solubility of S is increased by the presence of L. Type A diagrams indicate the formation of a soluble complex between S and L. If the slope of an A_{L} type diagram is greater than unity, then at least one component has a concentration that is greater than one. A slope of less than unity indicates a 1:1 stoichiometry between components S and L. The association constant (Kc) for complex formation can be calculated from Equation (1), where Sₜ represents the concentration of dissolved S:

$${K}_{c} = \frac{Slope}{{S}_{t} ( 1 - Slope )} \qquad Equation ( 1 )$$

FIG. 19 shows the phase solubility diagram of HP-B-CD and Niacinamide. It shows a linear increase in solubility and is classified as A_{L} type by the Higuchi and Connors classification. This demonstrates the formation of a soluble complex between HPBCD and niacinamide. The slope of the graph is less than one (slope=4.44×10⁻¹) which indicates a 1:1 stoichiometry of the complex. The association constant (Kc) for complex formation was found to be 79.856×10⁻² M⁻¹ and was calculated using Eq. (1). The absorbance was measured by UV at λ=217 nm.

FIG. 20 shows the phase solubility diagram of HPBCD and CBD. This diagram shows a linear increase in solubility and is classified as AL type by the Higuchi and Connors classification. This demonstrates the formation of a soluble complex between HPBCD and CBD. The slope of the graph is less than one (slope=2.97×10⁻¹) which indicates a 1:1 stoichiometry of the complex. The association constant (Kc) for complex formation was found to be 42.247×10⁻² M⁻¹ and was calculated using Eq. (1). The absorbance was measured by UV at λ=280 nm.

FIG. 21 shows the phase solubility diagram of HPBCD and pycnogenol. It shows a linear increase in solubility and is classified as A_{L} type by the Higuchi and Connors classification. This demonstrates the formation of a soluble complex between HPBCD and pycnogenol. The slope of the graph is greater than one (slope=15.87×10⁻¹) which indicates that the stoichiometry of the complex is not 1:1. The association constant (Kc) for complex formation was found to be 270.358×10⁻²M⁻¹ and was calculated using Eq. (1). The absorbance was measured by UV at λ=365 nm.

FIG. 22 shows the phase solubility diagram of HPBCD and tetrahydrocurcumin 1. It shows a linear increase in solubility and is classified as AL type by the Higuchi and Connors classification. This demonstrates the formation of a soluble complex between HPBCD and tetrahydrocurcumin. The slope of the graph is greater than one (slope=12.84×10⁻¹) which indicates that the stoichiometry of the complex is not 1:1. The association constant (Kc) for complex formation was found to be 452.113×10⁻² M⁻¹ and was calculated using Eq. (1). The absorbance was measured by UV at λ=280 nm.

FIG. 23 shows the phase solubility diagram of HPBCD and tamanu oil. This diagram shows a linear increase in solubility and is classified as AL type by the Higuchi and Connors classification. This demonstrates the formation of a soluble complex between HPBCD and tamanu oil. The slope of the graph is greater than one (slope=14.83×10⁻¹) which indicates that the stoichiometry of the complex is not 1:1. The association constant (Kc) for complex formation was found to be 307.039×10⁻² M⁻¹ and was calculated using Eq. (1). The absorbance was measured by UV at λ=266 nm.

FIG. 24 shows the phase solubility diagram of HPBCD and minoxidil. This diagram shows an initial linear increase in solubility followed by the formation of a plateau. The plateau indicates complete solubilization of minoxidil that additional amounts of HPBCD does not alter. This diagram is still considered as A type by the Higuchi and Connors classification. Since the graph is not linear, the slope does not give an accurate indication of the stoichiometry. The slope of the linear part of the graph was used to calculate the association constant (slope=11.249). The association constant (Kc) for complex formation was found to be 109.757×10⁻²M⁻¹ and was calculated using Eq. (1). The absorbance was measured by UV at λ=290 nm.

FIG. 25 shows the phase solubility diagram of HPBCD and benzocaine. This diagram shows an initial linear increase in solubility followed by the formation of a plateau. The plateau indicates complete solubilization of benzocaine that additional amounts of HPBCD does not alter. This diagram is still considered as A type by the Higuchi and Connors classification. Since the graph is not linear, the slope does not give an accurate indication of the stoichiometry. The slope of the linear part of the graph was used to calculate the association constant (slope=33.256). The association constant (Kc) for complex formation was found to be 103.100×10⁻² M⁻¹ and was calculated using Eq. (1). The absorbance was measured by UV at λ=305 nm.

### Example 4. Degradation Studies

The degradation kinetics of a zero-order reaction does not depend on the concentration of the reagents. Therefore, the rate of reaction (k)=−d[C]/dt, where [C] indicates decreasing concentration of reagent and t indicates time. Integration of the rate equation between initial concentration at time t=0 (C0) and concentration after time t=t (Ct) yields the equation Ct=C0−kt. When this linear equation is plotted according to FIG. 26, with concentration on the x vertical axis and time on the y horizontal axis, the slope of the graph is equal to −k.

Three molar concentrations of phosphoric acid (0.025 M, 0.05 M, and 0.1 M H₃PO₄) were added to HPBCD pycnogenol solution in deionized water at 25° C. The absorbance was measured at the selected time points, and the concentration was calculated. The degradation graph (FIG. 27) shows a zero-order kinetic reaction in the presence of phosphoric acid. The rate constants for the reaction of each H₃PO₄ concentration with HPBCD pycnogenol complex were calculated and included in Table 12. The wavelength for analysis was 275 nm.

TABLE 12
Rate Constants for HPBCD Pycnogenol Complex Degradation

| | k (rate constant) |
|---|---|
| HPBCD Pycnogenol Complex + 0.025M H₃PO₄ | 1.4967 × 10⁻⁴ |
| HPBCD Pycnogenol Complex + 0.05M H₃PO₄ | 1.4336 × 10⁻⁴ |
| HPBCD Pycnogenol Complex + 0.1M H₃PO₄ | 1.3888 × 10⁻⁴ |

Three molar concentrations of phosphoric acid (0.025 M, 0.05 M, and 0.1 M H₃PO₄) are added to HPBCD niacinamide solution in deionized water at 25° C. The absorbance was measured at the selected time points, and the concentration was calculated. The degradation graph (FIG. 28) shows a zero-order kinetic reaction in the presence of phosphoric acid. The rate constants for the reaction of each H₃PO₄ concentration with HPBCD niacinamide complex were calculated and included in Table 13. The wavelength for analysis was 265 nm.

TABLE 13
Rate Constants for HPBCD Niacinamide Complex Degradation

| | k (rate constant) |
|---|---|
| HPBCD Niacinamide Complex + 0.025M H₃PO₄ | 2.0293 × 10⁻³ |
| HPBCD Niacinamide Complex + 0.05M H₃PO₄ | 2.4150 × 10⁻³ |
| HPBCD Niacinamide Complex + 0.1M H₃PO₄ | 2.8666 × 10⁻³ |

Three molar concentrations of phosphoric acid (0.025 M, 0.05 M, and 0.1 M H₃PO₄) are added to HPBCD tamanu oil solution in deionized water at 25° C. The absorbance was measured at the selected time points, and the concentration was calculated. The degradation graph (FIG. 29) shows a zero-order kinetic reaction in the presence of phosphoric acid. The rate constants for the reaction of each H₃PO₄ concentration with HPBCD tamanu oil complex were calculated and included in Table 14. The wavelength for analysis was 266 nm.

TABLE 14
Rate Constants for HPBCD Tamanu Oil Complex Degradation

| | k (rate constant) |
|---|---|
| HPBCD Tamanu Oil Complex + 0.025M H₃PO₄ | 1.2422 × 10⁻⁴ |
| HPBCD Tamanu Oil Complex + 0.05M H₃PO₄ | 1.7098 × 10⁻⁴ |
| HPBCD Tamanu Oil Complex + 0.1M H₃PO₄ | 1.7240 × 10⁻⁴ |

Three molar concentrations of phosphoric acid (0.025 M, 0.05 M, and 0.1 M H₃PO₄) are added to HPBCD tetrahydrocurcumin solution in deionized water at 25° C. The absorbance was measured at the selected time points, and the concentration was calculated. The degradation graph (FIG. 30) shows a zero-order kinetic reaction in the presence of phosphoric acid. The rate constants for the reaction of each H₃PO₄ concentration with HPBCD tetrahydrocurcumin complex were calculated and included in Table 15. The wavelength for analysis was 280 nm.

TABLE 15
Rate Constants for HPBCD Tetrahydrocurcumin
Complex Degradation

| | k (rate constant) |
|---|---|
| HPBCD Tetrahydrocurcumin | 0.7346 × 10⁻⁴ |
| Complex + 0.025M H₃PO₄ | |
| HPBCD Tetrahydrocurcumin | 0.8150 × 10⁻⁴ |
| Complex + 0.05M H₃PO₄ | |
| HPBCD Tetrahydrocurcumin | 0.8386 × 10⁻⁴ |
| Complex + 0.1M H₃PO₄ | |

Three molar concentrations of phosphoric acid (0.025 M, 0.05 M, and 0.1 M H₃PO₄) are added to HPBCD minoxidil solution in deionized water at 25° C. The absorbance was measured at the selected time points, and the concentration was calculated. The degradation graph (FIG. 31) shows a zero-order kinetic reaction in the presence of phosphoric acid. The rate constants for the reaction of each H₃PO₄ concentration with HPBCD minoxidil complex were calculated and included in Table 16. The wavelength for analysis was 280 nm.

TABLE 16
Rate Constants for HPBCD Minoxidil Complex Degradation

| | k (rate constant) |
|---|---|
| HPBCD Minoxidil Complex + 0.025M H₃PO₄ | 0.6448 × 10⁻⁴ |
| HPBCD Minoxidil Complex + 0.05M H₃PO₄ | 0.6908 × 10⁻⁴ |
| HPBCD Minoxidil Complex + 0.1M H₃PO₄ | 0.7093 × 10⁻⁴ |

Three molar concentrations of phosphoric acid (0.025 M, 0.05 M, and 0.1 M H₃PO₄) are added to HPBCD benzocaine solution in deionized water at 25° C. The absorbance was measured at the selected time points, and the concentration was calculated. The degradation graph (FIG. 32) shows a zero-order kinetic reaction in the presence of phosphoric acid. The rate constants for the reaction of each H₃PO₄ concentration with HPBCD benzocaine complex were calculated and included in Table 17. The wavelength for analysis was 260 nm.

TABLE 17
Rate Constants for HPBCD Benzocaine Complex Degradation

| | k (rate constant) |
|---|---|
| HPBCD Benzocaine Complex + 0.025M H₃PO₄ | 1.2086 × 10⁻³ |
| HPBCD Benzocaine Complex + 0.05M H₃PO₄ | 1.3625 × 10⁻³ |
| HPBCD Benzocaine Complex + 0.1M H₃PO₄ | 1.2593 × 10⁻³ |

Three molar concentrations of phosphoric acid (0.025 M, 0.05 M, and 0.1 M H₃PO₄) are added to HPBCD CBD solution in deionized water at 25° C. The absorbance was measured at the selected time points, and the concentration was calculated. The degradation graph (FIG. 33) shows a zero-order kinetic reaction in the presence of phosphoric acid. The rate constants for the reaction of each H₃PO₄ concentration with HPBCD CBD complex were calculated and included in Table 18. The wavelength for analysis was 278 nm.

TABLE 18
Rate Constants for HPBCD CBD Complex Degradation

| | k (rate constant) |
|---|---|
| HPBCD CBD Complex + 0.025M H₃PO₄ | 1.5195 × 10⁻³ |
| HPBCD CBD Complex + 0.05M H₃PO₄ | 1.2591 × 10⁻³ |
| HPBCD CBD Complex + 0.1M H₃PO₄ | 1.5814 × 10⁻³ |

### Example 5—Content Uniformity

Content uniformity of the active in the HPBCD complexes was investigated by active recovery studies where known quantities of the active and active-HPBCD complex were dissolved in a 10 ml mobile phase to get a clear solution. The solution was further diluted with mobile phase and buffer before analyzing using HPLC. Tables 19-25 show the results of this analysis for each of the HPBCD complexes.

TABLE 19
Content Uniformity of HPBCD Tetrahydrocurcumin Complex

| Starting Material | Recovery % | Avg. Recovery % |
|---|---|---|
| 0.50 mg | 96.15 | 98.07 |
| 0.50 mg | 100.00 | |
| 0.50 mg | 98.04 | |
| 0.50 mg | 96.16 | |
| 0.50 mg | 99.99 | |

TABLE 20
Content Uniformity of HPBCD Niacinamide Complex

| Starting Material | Recovery % | Avg. Recovery % |
|---|---|---|
| 0.90 mg | 98.86 | 99.12 |
| 0.90 mg | 99.54 | |
| 0.90 mg | 99.01 | |
| 0.90 mg | 99.68 | |
| 0.90 mg | 98.49 | |

TABLE 21
Content Uniformity of HPBCD Pycnogenol Complex

| Starting Material | Recovery % | Avg. Recovery % |
|---|---|---|
| 0.20 mg | 99.01 | 96.16 |
| 0.20 mg | 94.34 | |
| 0.20 mg | 92.59 | |
| 0.20 mg | 100.50 | |
| 0.20 mg | 94.35 | |

TABLE 22
Content Uniformity of HPBCD Minoxidil Complex

| Starting Material | Recovery % | Avg. Recovery % |
|---|---|---|
| 0.60 mg | 98.36 | 98.41 |
| 0.60 mg | 101.69 | |
| 0.60 mg | 96.77 | |
| 0.60 mg | 100.00 | |
| 0.60 mg | 95.24 | |

TABLE 23
Content Uniformity of HPBCD Benzocaine Complex

| Starting Material | Recovery % | Avg. Recovery % |
|---|---|---|
| 0.50 mg | 96.82 | 97.82 |
| 0.50 mg | 97.64 | |
| 0.50 mg | 98.54 | |
| 0.50 mg | 96.92 | |
| 0.50 mg | 99.16 | |

TABLE 24
Content Uniformity of HPBCD Tamanu Oil Complex

| Starting Material | Recovery % | Avg. Recovery % |
|---|---|---|
| 0.20 mg | 86.96 | 94.88 |
| 0.20 mg | 100.00 | |
| 0.20 mg | 95.24 | |
| 0.20 mg | 86.95 | |
| 0.20 mg | 105.26 | |

TABLE 25
Content Uniformity of HPBCD CBD Complex

| Starting Material | Recovery % | Avg. Recovery % |
|---|---|---|
| 0.10 mg | 94.34 | 93.59 |
| 0.10 mg | 90.91 | |
| 0.10 mg | 100.00 | |
| 0.10 mg | 92.59 | |
| 0.10 mg | 90.09 | |

### Example 6 FTIR Studies

FIG. 34 shows FTIR spectrum of HPBCD. The region from 700-1200 cm-1 shows peaks due to the C—O—C bending, C—C—O stretching, and skeletal vibration involving the α-1,4 linkage. The region from 1200-1500 cm⁻¹ shows peaks due to C—H and O—H bending. The small broad peak at 1650 cm⁻¹ is the H—O—H bending peak due to water of crystallization of water molecules trapped within the cavity of the cyclodextrin molecule. The region of 2850-3000 cm⁻¹ is the C—H stretch and the strong broad peak at 3300 cm⁻¹ is the O—H stretch.

FIG. 35 shows overlaid FTIR spectra for benzocaine (red), HPBCD (green), and HPBCD benzocaine inclusion complex (blue). The spectrum of the inclusion complex mirrors the spectrum of HPBCD, which indicates that the benzocaine molecule entered the cavity of the cyclodextrin. The N—H amine group stretching peaks in the 3200-3500 cm⁻¹ region of benzocaine disappeared, as well as the aromatic peaks from the benzene ring (3000 cm⁻¹ and 1300-1500 cm⁻¹), indicating insertion of this portion of the molecule within the HPBCD cavity. The peaks from the complex spectrum at 1690 cm⁻¹ (C═O stretch), 1600 cm⁻¹ (C—C stretch), 1520 cm⁻¹ (C—H bend), and 1290 cm-1 (C—O—C stretch) correspond to the ethyl ester portion of the benzocaine molecule which is outside the cyclodextrin cavity. The small broad peak at 1650 cm⁻¹ (H—O—H bending) is the water of crystallization peak and indicates that there are a few water molecules trapped within the cavity of the HPBCD benzocaine complex. The absence of new peaks in the spectrum of the inclusion complex indicates a non-covalent interaction between the host and guest molecule.

FIG. 36 shows overlaid FTIR spectra for CBD (red), HPBCD (green), and HPBCD CBD inclusion complex (blue). A sizeable portion of the CBD molecule hangs outside the cyclodextrin cavity. The region from 700-1200 cm⁻¹ shows peaks due to the C—O—C bending, C—C—O stretching, and skeletal vibration involving the α-1,4 linkage of HPBCD, and the spectra of the complex mirrors this region. The 1:1 molar ratio of HPBCD to CBD only allows one ring of the CBD molecule to enter the cyclodextrin cavity, thus there is a large portion of the CBD molecule hanging outside the HPBCD. The complex spectral region from 2800-3550 cm⁻¹ shows characteristic peaks for both HPBCD and CBD. The peaks at 3520 cm⁻¹ (O—H stretch) and 3400 cm⁻¹ (O—H stretch) are from the hydroxyl groups off the benzene ring of CBD, and the small broad peak at 3300 cm⁻¹ (O—H stretch) comes from HPBCD. The quartet of peaks starting at 2800 cm-1 and ending at 2980 cm⁻¹ are asymmetrical stretching vibrations of —CH2 bonds, which comes from the C5 chain attached to the benzene ring in the CBD molecule. The small broad peak at 1650 cm⁻¹ (H—O—H bending) in the HPBCD spectrum is the water of crystallization peak. The absence of this peak in the spectrum of the complex indicates that there are no water molecules trapped within the cavity of the HPBCD CBD complex. The medium sharp peaks at 1620 cm⁻¹, 1580 cm⁻¹, 1510 cm⁻¹ and 1440 cm⁻¹ (C—C stretch) are the aromatic ring stretching vibrations from the benzene ring of CBD. The small broad peaks in the complex spectral region from 1240-1400 cm⁻¹ show peaks due to C—H and O—H bending of the rings. The sharp peak at 1210 cm⁻¹ (C—O stretch) is due to the hydroxyl group off the benzene ring in CBD. The small sharp peak at 900 cm⁻¹ (C—H bend) is from the alkene bond attached to the ring of the CBD molecule, which lies outside the HPBCD cavity. The absence of new peaks in the spectrum of the inclusion complex indicates a non-covalent interaction between the host and guest molecule.

FIG. 37 shows overlaid FTIR spectra for minoxidil (green), HPBCD (blue), and HPBCD minoxidil inclusion complex (red). The spectrum of the inclusion complex mirrors the spectrum of HPBCD and indicates that the minoxidil molecule is fully incorporated into the cavity of the cyclodextrin. The aromatic peaks from the aminopyrimidine and piperidine rings (1200-1700 cm⁻¹) of minoxidil are absent from the spectrum of the complex, indicating insertion within the HPBCD cavity. The 2:1 molar ratio of HPBCD to minoxidil allows both rings of the minoxidil molecule to be incorporated into two molecules of HPBCD, thus none of the minoxidil molecule is outside the cyclodextrin cavity. The small broad peak at 1650 cm⁻¹ (H—O—H bending) is the water of crystallization peak and indicates that there are a few water molecules trapped within the cavity of the HPBCD minoxidil complex. The absence of new peaks in the spectrum of the inclusion complex indicates a non-covalent interaction between the host and guest molecule.

FIG. 38 shows overlaid FTIR spectra for niacinamide (green), HPBCD (blue), and HPBCD niacinamide inclusion complex (red). The spectrum of the inclusion complex mirrors the spectrum of HPBCD, which indicates that the niacinamide molecule entered the cavity of the cyclodextrin moiety. The aromatic peaks from the pyridine ring (1200-1500 cm¹) are absent from the spectrum of the complex, indicating insertion of this portion of the molecule within the HPBCD cavity. The peaks from the complex spectra at 1695 cm-1 (C═O stretch), 1610 cm⁻¹ (N—H bend) and 1600 cm⁻¹ (N—H bend) correspond to the amide portion of the niacinamide molecule which is outside the cyclodextrin cavity. The small broad peak at 1650 cm⁻¹ (H—O—H bending) in the HPBCD spectrum is the water of crystallization peak. The absence of this peak in the spectrum of the complex indicates that there are no water molecules trapped within the cavity of the HPBCD niacinamide complex. The absence of new peaks in the spectrum of the inclusion complex indicates a non-covalent interaction between the host and guest molecule.

FIG. 39 shows overlaid FTIR spectra for pycnogenol (green), HPBCD (blue), and HPBCD pycnogenol inclusion complex (red). The spectrum of the inclusion complex mirrors the spectrum of HPBCD, which indicates that the pycnogenol molecule entered the cavity of the cyclodextrin. The 3:1 molar ratio of HPBCD to pycnogenol allows three of the rings of the procyanidin or proanthocyanidin molecule to be incorporated within the cavity of three cyclodextrin molecules. The fourth ring from the procyanidin and proanthocyanidin moieties of pycnogenol lies outside the cavity of HPBCD. The peaks from the complex spectra at 1700 cm⁻¹ (C═C stretch), 1600 cm⁻¹ (C—C stretch) and 1510 cm⁻¹ (C—C stretch) correspond to the aromatic stretching of the benzene and dihydropyran rings. The peaks at 1300 cm⁻¹ (C—O stretch) and 1250 cm⁻¹ (C—O stretch) correspond to the alcohol groups off the benzene ring. The small broad peak at 1650 cm⁻¹ (H—O—H bending) in the HPBCD spectrum is the water of crystallization peak. The absence of this peak in the spectrum of the complex indicates that there are no water molecules trapped within the cavity of the HPBCD pycnogenol complex. The absence of new peaks in the spectrum of the inclusion complex indicates a non-covalent interaction between the host and guest molecule.

FIG. 40 shows overlaid FTIR spectra for tamanu oil (green), HPBCD (blue), and HPBCD tamanu oil inclusion complex (red). The spectrum of the inclusion complex mirrors the spectrum of HPBCD, which indicates that the tamanu oil entered the cavity of the cyclodextrin. Tamanu oil is made up of the C16 and C18 fatty acids oleic, linoleic, palmitic and stearic. The 3:1 molar ratio of HPBCD to tamanu oil allows for most of the fatty acid carbon chains to be incorporated within the cyclodextrin cavity. The peaks from the complex spectra at 2915 cm⁻¹ (C—H stretch) and 2865 cm⁻¹ (C—H stretch) are asymmetrical stretching vibrations of the —CH2 bonds from the portion of the fatty acid hanging outside the cavity of HPBCD. The carboxylic acid headgroup of the fatty acid also lies outside the cyclodextrin cavity, with the carbonyl peak in the complex spectra occurring at 1750 cm⁻¹ (C═O stretch). The very small broad peak at 1650 cm⁻¹ (H—O—H bending) is the water of crystallization peak and indicates that most of the water molecules trapped within the cavity of the HPBCD were replaced by tamanu oil in the complex. The strong broad peak at 3300 cm⁻¹ (O—H stretch) in HPBCD is much smaller and broader in the complex, and this could indicate weak interaction between the —OH group of the fatty acid and the —OH group of the HPBCD ring.

FIG. 41 shows overlaid FTIR spectra for tetrahydrocurcumin (green), HPBCD (blue), and HPBCD tetrahydrocurcumin inclusion complex (red). The spectrum of the inclusion complex mirrors the spectrum of HPBCD, which indicates that the tetrahydrocurcumin molecule entered the cavity of the cyclodextrin. The aromatic peaks from the benzene rings (1100-1400 cm¹) and the strong carbonyl peak (1600 cm¹) are absent from the spectrum of the complex, indicating insertion of these portions of the molecule within the HPBCD cavity. The 3:1 molar ratio of HPBCD to tetrahydrocurcumin allows both rings of the tetrahydrocurcumin molecule, as well as the carbonyl groups to be incorporated into three molecules of HPBCD. The peaks from the complex spectra at 1300 cm⁻¹ (C—O—C stretch), 1290 cm⁻¹ (C—O—C stretch), 810 cm⁻¹ (C—H stretch) and 800 cm′ (C—H stretch) correspond to the methoxy groups off the benzene rings, and the peak at 1510 cm⁻¹ (C—C stretch) corresponds to the small part of the carbon linkage in the tetrahydrocurcumin molecule, which lie outside the cyclodextrin cavity. The small broad peak at 1650 cm⁻¹ (H—O—H bending) in the HPBCD spectrum is the water of crystallization peak. The shift of this broad peak to 1620 cm⁻¹ in the spectrum of the complex indicates that there is hydrogen bonding occurring between the water molecules trapped within the cavity and the alcohol group of the tetrahydrocurcumin. The absence of new peaks in the spectrum of the inclusion complex indicates a non-covalent interaction between the host and guest molecule.

### Example 6. Permeation Studies of Hydroxypropyl β-Cyclodextrin Formulations

Four cream formulations comprising HPBCD have been developed with each of four active components (“Actives”). The four creams are:

i. Scar Reduction Cream with tamanu oil as the active component.

ii. Pain Relief Cream with cannabidiol (CBD) as the active component.

iii. Nourishing Cream with niacinamide (NA) as the active component.

iv. Brightening Cream with tetrahydrocurcumin (TC) as the active component.

Eight formulations were prepared. These comprised four creams with the addition of an HPBCD complexed Active and four creams with a non-complexed Active (no addition of HPBCD). Three pairs of creams have single active ingredients, namely CBD, NA, and TC, for pain relief, nourishing, and brightening creams respectively. The fourth pair contained tamanu oil, which is comprised of the eighteen carbon fatty acids linoleic acid (LA), oleic acid (OA), and stearic acid (SA), and the sixteen carbon fatty acid palmitic acid (PA).

Semi-solid cream formulations were prepared by simple emulsification of 4% Jeesperse ICE-T CCPS (emulsifier) comprising (INCI) cetearyl alcohol, behentrimonium chloride, and polyquaternium-37; 1% Jeecide AA (preservative) comprising (INCI) benzyl alcohol, benzoic acid and sorbic acid; an Active, and water up to 100%, which creates an emulsion without heat. The term “(INCI)” stands for the International Nomenclature of Cosmetic Ingredients; INCI names are mandated on the ingredient statement of every consumer personal care product. An Active complexed with HBPCD or a non-complexed Active was added. Complexed CBD and Tamanu oil represented 10% w/w of the composition; TC and niacinamide represented 3% w/w of the composition.

TABLE 26
Formulations

| Sample | Formulation | | Active |
|---|---|---|---|
| No. | ID | Composition | Ingredients |
| 1 | Scar | 47% Active in cream | Hydroxypropyl β- |
| | Reduction | (Equal to 10% | cyclodextrin/ |
| | Cream | Tamanu Oil) | Tamanu Oil |
| | | | Inclusion Complex |
| 2 | Scar | 10% Total Tamanu oil | Tamanu Oil |
| | Reduction | in cream | |
| | Cream | | |
| 3 | Pain | 33% Active in cream | Hydroxypropyl β- |
| | Relief | (Equal to 10% CBD) | cyclodextrin/CBD |
| | Cream | | Inclusion Complex |
| 4 | Pain | 10% Total CBD | CBD |
| | Relief | in cream | |
| | Cream | | |
| 5 | Nourishing | 34% Active in cream | Hydroxypropyl β- |
| | Cream | (Equal to 3% | cyclodextrin/ |
| | | Niacinamide) | Niacinamide |
| | | | Inclusion Complex |
| 6 | Nourishing | 3% Total Niacinamide | Niacinamide |
| | Cream | in cream | |
| 7 | Brightening | 33% Active in cream | Hydroxypropyl β- |
| | Cream | (Equal to 3% | cyclodextrin/ |
| | | Tetrahydrocurcumin) | Tetrahydrocurcumin |
| | | | Inclusion Complex |
| 8 | Brightening | 3% Tetrahydrocurcumin | Tetrahydrocurcumin |
| | Cream | in cream | |

The pH and viscosity of the cream compositions containing Active are shown in Table 27 below.

TABLE 27
pH and viscosity of the cream compositions

| Cream | | Viscosity |
|---|---|---|
| Composition | pH | (5.0 rpm@96° F.) |
| Scar Reduction | 3.25 | cP120000 |
| with Tamanu Oil | | |
| Scar Reduction with | 3.66 | cP 183000 |
| Complexed Tamanu Oil | | |
| Pain Relief with CBD | 3.11 | cP 101000 |
| Pain Relief with | 4.08 | cP 117000 |
| Complexed CBD | | |
| Nourishing Cream | 4.97 | cP 48000 |
| with Niacinamide | | |
| Nourishing Cream with | 4.97 | cP 81560 |
| Complexed Niacinamide | | |
| Brightening Cream with | 3.46 | cP 104000 |
| Tetrahydrocurcumin | | |
| Brightening Cream | 4.23 | cP 43500 |
| with Complexed | | |
| Tetrahydrocurcumin | | |

Skin Permeation and Delivery

The test formulations are creams, because the cream vehicle sits on the skin, and only the active penetrates.

Test Device:

Skin permeation was evaluated using a custom-fabricated Franz-type vertical diffusion cell (FDC). The basic configuration of the device includes (a) a donor compartment for applying a test formulation to a membrane where the Active released must permeate; (b) a piece of skin, about 2.5 cm×2.5 cm square, mounted over a receptor well, (b) a receptor well or compartment fully filled with a receptor fluid (PBS containing 0.1% w/w sodium azide as a preservative and ≤4% bovine serum albumin (BSA) (or with ≤4% w/w HPBCD, PEG400 or Brij020) to ensure uniform contact with the underside of the skin piece. Fluid samples can be withdrawn for analysis from the receptor fluid.

The membrane was split thickness human cadaver skin (250μ-300μ thick) obtained from the posterior leg of a 66 year old white male. The cadaver skin was taken within 24 hr post-mortem and flash frozen. Membranes were defrosted, washed and subjected to visual inspection before use.

Skin integrity was tested by assaying transepidermal electrical resistance to an alternating current (TEER) (impedance). An aliquot of 150 μl of PBS was introduced into each diffusion cell donor well. After 10 minutes, a blunt electrode probe was placed into the donor well. A second electrode was then inserted into receptor fluid via the sample port on the receptor chamber of the FDC. An alternating current (“AC”) signal, 100 mV root mean square (“RMS”) at 100 Hz, was then applied across the skin using a waveform generator. The impedance was measured with a digital multimeter and the results recorded in kΩ Membranes that deviated from average were rejected.

Skin delivery and permeation studies were performed on six (6) replicates per Active formulation. A finite dose was applied to the surface of the skin under nonocclusive conditions. Dose volume was 10 μl (18 mg/cm²). The dose was spread using a blunt glass rod.

Receptor chambers were inserted in a dry block with an external magnetic stir bar drive that accommodated up to 15 Franz cells per block. Receptor wells were stirred at about 300 rpm without vortex. Receptor well temperature was maintained at 32±0.5° C.; skin surface temperature was maintained at 30±1.0° C.

Receptor wells were sampled at three time points, namely 8 hr, 24 hr and 48 hr; 300 μl was removed, loaded in a 96-well plate, and stored at 4-8° C. prior to analysis. Samples were analyzed within 5 days of collection. There was no further preparation of the samples prior to analysis.

Retention Sampling

At the final time point, the membrane was washed by contact with 200 μL water-EtOH (50-50) for 5 minutes, which then was removed with a KimWipe®. The membrane was tapestripped 3× to remove stratum corneum layers and then discarded. The epidermis-dermis layers were separated on a 60° C. hotplate for 1 minute (where necessary). The epidermis was extracted with 3 mL extraction fluid at 40° C. for 24 hours with gentle agitation. The dermis was extracted with 3 ml extraction fluid at 40° C. for 24 hours with gentle agitation.

Transdermal flux of each Active was calculated by measuring the concentration of Active in de-aerated isotonic phosphate buffered saline solution (PBS) at pH 7.4 containing 0.01% NaN3 (a preservative) and up to 4% bovine serum albumin (BSA) or HPBCD, PEG400, or Brij98 at four, eight, and twenty-four hours. Retention of the Actives in the epidermis and delivery of Actives to the dermis was measured by extracting the Active from each layer individually at twenty-four hours using dimethyl sulfoxide (DMSO).

Analytical Methods

Actives were quantified by Liquid Chromatography—Mass Spectrometry (LC-MS) or UV detection on an Agilent 1260 with an Agilent G6120 LC-MS detector or G4212B diode array detector. (The oleic acid constituent of tamanu oil, which is the main constituent, was quantified without resolving the individual fatty acids of the tamanu oil.

Preparation of Mobile Phases

Mobile Phase A: Mobile Phase A was prepared by transferring 1.0 ml of formic acid (Fisher A117-50) into a 2 L media bottle 1 L of LC/MS grade water (Fisher: W6-4) was then measured in a volumetric cylinder and the contents transferred into the 2 L media bottle. The mixture in the media bottle was shaken until the contents were fully mixed. Mobile Phase A was stored for less than a week during the course of the analysis.

Mobile Phase B:

Mobile Phase B either consisted of 100% LC/MS grade methanol (Fisher A456-4) used as is, or consisted of methanol with 0.1 vol % formic acid (Fisher: A117-50). For the latter combination, the mobile phase was prepared by transferring 1.0 ml of formic acid into a 2 L media bottle. 1 L of LC/MS grade methanol was then measured in a volumetric cylinder and the contents transferred into the 2 L media bottle. The mixture in the media bottle was shaken until the contents were fully mixed. Mobile Phase B was stored for less than one week during the course of the analysis.

Preparation of Calibration Standards

Individual calibration standards were prepared for each Active. An Active Stock Solution was prepared by first weighing 4 mg of the Active with an analytical balance in a glass vial. The vial was then tared on the balance and 4 ml of a Diluent (water for NA, and dimethyl sulfoxide (DMSO) for CBD, TC and Oleic acid) was then introduced into the glass vial with a pipettor. The vial was reweighed, removed from the analytical balance, and capped. The capped vial was vortexed and sonicated using an ultrasonication bath until the Actives were fully dissolved. Calibration standards were then prepared by serial dilution 5 fold with the diluent. Standards Ca13-Call were used to prepare calibration curves. The concentration of Active in each of the calibration standards is shown in Table 28 below:

TABLE 28
Calibration standards

| Calibration Standard | Concentration (μg/ml) |
|---|---|
| Stock Solution | 1000 |
| Cal 2 | 200 |
| Cal 3 | 40 |
| Cal 4 | 8 |
| Cal 5 | 1.6 |
| Cal 6 | 0.32 |
| Cal 7 | 0.64 |
| Cal 8 | 0.0128 |

Table 29: shows the chromatographic parameters for detection of each Active

TABLE 29
shows the chromatographic parameters for detection of each Active

| | Niacinamide | Oleic Acid | Tetrahydrocurcumin | Cannabidiol |
|---|---|---|---|---|
| Column | Agilent Poroshell | Agilent G6120 LC-MS | Agilent Zorbax Eclipse | Agilent Zorbax Eclipse |
| | EC-C18, 2.1 × 150 μm, | | PAH, 2.1 × 100 μm, | PAH, 2.1 × 100 μm, |
| | 4.0 μm | | 3.5 μm | 3.5 μm |
| Mobile phase | A: water with 0.1% | A: water with 0.1% | A: water with 0.1% | A: water with 0.1% |
| | formic acid | formic acid | formic acid | formic acid |
| | B: methanol | B: methanol with 0.1% | B: methanol with 0.1% | B: methanol with 0.1% |
| | | formic acid | formic acid | formic acid |
| Flow rate: | 0.2 ml/min | 0.8 ml/min | 1.0 ml/min | 1.0 ml/min |
| Column temperature: | 40° C. | 30° C. | 30° C. | 30° C. |
| UV detection: | 260 nm | 215 nm | 280 nm | 280 nm |
| Injection volume: | 10 μl | 10 μl | 10 μl | 10 μl |
| Retention time: | ≈2.2 min | ≈2.0 min | ≈2.55 min | ≈3.9 min |

Representative chromatographs of high performance liquid chromatography (HPLC) calibration standards for niacinamide (FIG. 42), tamanu oil (FIG. 43), tetrahydrocurcumin (TC) (FIG. 44) and cannabidiol (CBD) (FIG. 45) are shown. The y-axis of each chromatogram is a measure of the intensity of absorbance (in units of mAU, or milli-Absorbance Units). The x-axis is in units of time (minutes), and is used to determine the retention time (tR) for each peak. The main peak in the tamanu oil chromatogram is that of oleic acid.

### Calculation

After the LC-MS or UV testing was complete, samples were analyzed using ChemStation software (Agilent). The AUCs of the Active peaks were recorded and converted to μg/ml values using a calibration curve developed from the calibration standards' AUC values and known concentration values after dilution of the extracted media. The values in μg/mL are the amount extracted from the skin at various timepoints, These concentrations were then multiplied by the receptor volume (3.3 mL) or skin extraction volume (3.0 mL) and then divided by the surface area of the skin exposed to the receptor fluid (0.55 cm²) for an end cumulative amount in μg/cm². For receptor fluid time points greater than 8 hours, this μg/cm² value was corrected for the sample aliquot volumes removed to compensate for the dilution caused by replacing the sample volume with fresh buffer solution.

The results of skin integrity testing are shown in Table 30. The skin impedance value varies with the specific piece of skin used.

TABLE 30
Skin integrity TEER test results (Impedance)

| | Formulation | | | | | | | |
|---|---|---|---|---|---|---|---|---|
| | | | | | | 10% | | 10% |
| | | 3% | | | 10% | complexed | 10% | complexed |
| | 3% | Complexed | 3% | 3% | CBD | CBD | Tamanu | Tamanu |
| | TC | TC | Niacinamide | Complexed | Pain | Pain | Oil Scar | oil Scar |
| | Brightening | Brightening | Nourishing | Nourishing | Relief | Relief | Reduction | Reduction |
| Replicate | Cream | Cream | Cream | Cream | Cream | Cream | cream | Cream |
| 1 | 8.3 | 8.3 | 9.3 | 8.7 | 7.7 | 7.6 | 8.1 | 7.7 |
| 2 | 3.5 | 4.3 | 3.3 | 3.4 | 7.5 | 7.6 | 4.9 | 5.9 |
| 3 | 2.8 | 2.6 | 3 | 3 | 2.3 | 2.2 | 2.5 | 2.4 |
| 4 | 4.4 | 4.2 | 7.2 | 5.5 | 3.8 | 3.8 | 4.1 | 4.1 |
| 5 | 2.8 | 2.9 | 2.5 | 2.8 | 3.4 | 3.5 | 3.2 | 3.4 |
| 6 | 2.3 | 2.3 | 2.5 | 2.4 | 2.1 | 2 | 2.3 | 2.2 |

A transdermal graph is a plot of delivered dose (in μg/cm²) versus time elapsed (in hours). The delivered dose shown is the average of the results across the six replicates with the standard error of the mean. The Transdermal graph shows the amount of active present in the skin at the given timepoints (in μg/cm²).

Flux, with values in μg/cm2/hr is obtained by dividing the delivered dose by the amount of time (either 8, 24, or 48 hours). A Flux bar graph (plotting flux versus time elapsed (hours) shows the amount of active going through the skin at a given time (values in μg/cm2/hr)

A Skin Retention bar graph is a plot of delivered dose (μg/cm²) versus time (hrs). It shows the amount of active in the epidermis and the dermis after 48 hours (in μg/cm²).

Any sections of a graph that shows zero delivered dose implies that the active is sitting on top of the skin and is not penetrating through; for any such samples, a small amount was actually going through, but it was below the level of background noise and thus not included.

Nourishing Cream Containing Either Niacinamide (Molecular Weight 122.12 g/Mol) or a Niacinamide HBPCD Inclusion Complex)

Transdermal, flux, and skin retention graphs for the Active Niacinamide are shown in FIGS. 46A, 46B and 46C. Due in part to the strong water solubility of niacinamide, the data were highly variable. The transdermal graph shown in FIG. 46A and the flux graph in FIG. 46B show that more active is delivered through the skin in the non-complexed cream (from 8 hours to 48 hours). The complexed niacinamide, which is larger due to the presence of the cyclodextrin, is delivered through the skin at a steady rate from 8 hours to 48 hours. Without being limited by theory, it is possible that cyclodextrin slows the release of the active into the skin.

The skin retention graph in FIG. 46C shows that even with a lower flux through the skin and a lower overall delivered dose, the amount of niacinamide delivered to the dermis in the cyclodextrin complex is the same as for the non-complexed niacinamide. Therefore complexation with cyclodextrin is effective to increase the penetration depth of the niacinamide included active.

Pain Relief Cream Containing Either Cannabidiol (“CBD”, Molecular Weight 314.464 g/Mol) or a Cannabidiol HBPCD Inclusion Comples (is this Word Accurate???)

The size of the CBD molecule is comparatively large. The data for cannabidiol was less variable than that for niacinamide (save for one outlier removed with Dixon's Qtest); this is most likely due to the poor water solubility of CBD.

Each of the transdermal (FIG. 47A), flux (FIG. 47B) and skin retention (FIG. 47C) bar graphs for CBD show that from 0-8 hours no amount of CBD was detected as penetrating through the skin. The amount that did pass through if any was too low to be detected from the background noise.

The data shows that at time points of 24 and 48 hours, more CBD-cyclodextrin inclusion complex was detected transdermally (FIG. 47A) and fluxed through the skin (FIG. 47B) than for non-included CBD.

The data also shows that substantially more active was detected in the epidermis with the cyclodextrin-CBD cream versus the non-included CBD cream after 48 hours.

Based on the above data, we conclude that complexing a lipophilic material (such as CBD) with cyclodextrin enhances the ability of the active to penetrate the skin and increases the amount of active available to the epidermis and upper layers of the skin.

The amount of complexed CBD detected in the dermis was virtually the same as the amount of un-complexed CBD detected. This result may be attributed to the expected time-release capabilities of complexation with cyclodextrin.

Scar Reduction Cream Containing Either Tamanu Oil (Molecular Weight 873.4 g/Mol) or a Tamanu Oil HPBCD Inclusion Complex

Because oleic acid (molecule weight 282.417 g/mol) is the main constituent of tamanu oil, it was selected for analysis for the tamanu oil-cyclodextrin complex cream and the un-complexed tamanu oil cream.

The transdermal (FIG. 48A), flux (FIG. 48B) and skin retention (FIG. 48C) data show that virtually no amount of oleic acid is present transdermally at either 8 hours, 24 hours, or 48 hours; a small amount was detected but it was below background noise, and thus not included. This would imply that the majority of the oleic acid/tamanu oil remained on top of the skin.

After 48 hours, the transdermal (FIG. 48A) and skin retention (FIG. 48C) data show that the amount of active detected in the epidermis was larger for the un-complexed tamanu oil (oleic acid), while the amount of active detected in the dermis was larger for the tamanu oil-cyclodextrin complex. The skin retention bar graph (FIG. 48C) shows that the amount of oleic acid detected in the epidermis and the dermis for the non-complexed tamanu oil is virtually equivalent, while the amount of oleic acid detected in the dermis is substantially higher than in the epidermis for the complexed tamanu oil. The fact that less complexed tamanu oil was found in the epidermis shows that the cyclodextrin host allows the oil to fully penetrate the skin instead of just forming a film on the surface.

This data shows that complexation with cyclodextrin may increase depth of penetration of an oil, and that the cyclodextrin complex may deliver more active to the deeper layers of the skin.

Brightening Cream Containing Either Tetrahydrocurcumin (“TC”, Molecular Weight 372.417 g/Mol) or a Tetrahydrocurcumin-HBPCD Inclusion Complex

Tetrahydrocurcumin is the largest molecule tested in this study.

The amount of tetrahydrocurcumin detected transdermally is greater for the complexed TC than for the un-complexed TC at all analyzed timepoints (8 hours, 24 hours, 48 hours, epidermis, and dermis (FIG. 49A). Accordingly, cyclodextrin complexation increases the permeability and penetration of this large lipophilic material.

The flux data (FIG. 49B) shows that a large amount of active passed through the skin within the first 8 hours for the cyclodextrin-TC complex, whereas no un-complexed TC penetrated the skin within the first 8 hours. The flux slowed somewhat during 8 to 24 hours for the cyclodextrin-TC complex, and then increased again in the 24 to 48 hour period.

The skin retention data (FIG. 49C) shows that TC is retained in all layers of the skin. More of the complexed TC is retained in the epidermis versus the non-complexed TC. A greater concentration of complexed TC than the non-complexed is also retained in the dermis.

Overall we conclude that cyclodextrin complexation increases the bioavailability of an active ingredient when applied topically to the skin.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for improving incorporation of a guest compound in a cavity of a hydroxypropyl-β-cyclodextrin host comprising:
(a) establishing a vacuum in the cavity of the hydroxypropyl-β-cyclodextrin (HPBCD);
(b) adding the guest compound, wherein the guest compound is substantially free of a solvent;
(c) incorporating the guest compound into the cavity; and
(d) forming an active agent-hydroxypropyl-β-cyclodextrin inclusion complex.

2. The method according to claim 1, wherein the solvent is an aqueous solvent or an organic solvent.

3. The method according to claim 1, wherein the guest compound may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% included into the cavity of the cyclodextrin molecule.

4. The method according to claim 1, wherein a molar ratio of the guest compound to the HPBCD may be about 10:1, about 9:1, about 8:1, about 7:1, about 6:1, about 5:1, about 4:1, about 3:1, about 2:1, about 1:1 to about 1:300; i.e., about 1:1, about 1:2, about 1:3, about 1:4, about 1:5, about 1:6, about 1:7, about 1:8, about 1:9, about 1:10, about 1:11, about 1:12, about 1:13, about 1:14: about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:51, about 1:52, about 1:53, about 1:54, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1: 84, about 1:85, about 1:86, about 1:87, about 1:88, about 1:89, about 1:90, about 1:91, about 1:92, about 1:93, about 1:94, about 1:95, about 1:96, about 1:97, about 1: 98, about 1:99, about 1:100.

5. The method according to claim 1, wherein the guest compound is a lipophilic active agent.

6. The method according to claim 1, wherein the guest compound is selected from the group consisting of an anti-fungal agent, an anti-histamine agent; an anti-hypertensive agent; an anti-protozoal agent; an anti-oxidant; an anti-pruritic agent; an anti-skin atrophy agent; an anti-viral agent; a caustic agent; a calcium channel blocker; a cytokine-modulating agent; a prostaglandin analog; a chemotherapeutic agent; an irritant agent; a TRPC channel inhibitor agent; and a vitamin.

7. The method according to claim 1, further comprising combining a therapeutic amount of the active agent-inclusion complex with a pharmaceutically acceptable carrier; and forming a pharmaceutical composition.

8. The method according to claim 7, wherein the pharmaceutical composition is effective
(a) to reduce contact-based side effects compared to the active agent alone; or
(b) to improve bioavailability when compared to the bioavailability of the non-complexed active agent; or
(c) to improve stability of the active agent when compared to the stability of the non-complexed active agent alone; or
(d) to improve penetration of the active agent when compared to the penetration of the non-complexed active agent alone;
(e) to improve retention of the active agent in a targeted tissue when compared to the retention of the noncomplexed active agent alone; or
(f) to reduce toxicity of the active agent when compared to the toxicity of the non-complexed active agent alone; or
(g) to deliver a minimal effective concentration of the active agent to locations in vivo with a small amount of formulation volume.

9. The method according to claim 7 further comprising formulating the composition with a polymer,
(a) wherein the composition is characterized by slow release; or
(b) wherein the composition is characterized by controlled release; or
(c) wherein the composition is characterized by sustained release.

10. The method according to claim 1, further comprising combining a cosmetic amount of the active agent-inclusion complex with a cosmetically acceptable carrier; and forming a cosmetic composition.

11. The method according to claim 9, wherein the cosmetic composition may be effective
(a) to reduce contact-based side effects compared to the active agent alone; or
(b) to improve bioavailability when compared to the bioavailability of the non-complexed active agent; or
(c) to improve stability of the active agent when compared to the stability of the non-complexed active agent alone; or
(d) to improve penetration of the active agent when compared to the penetration of the non-complexed active agent alone;
(e) to improve retention of the active agent in a targeted tissue when compared to the retention of the noncomplexed active agent alone; or
(f) to reduce toxicity of the active agent when compared to the toxicity of the non-complexed active agent alone; or
(g) to deliver a minimal effective concentration of the active agent to locations in vivo with a small amount of formulation volume.

12. The method according to claim 9 further comprising formulating the composition with a polymer,
(a) wherein the composition is characterized by slow release; or
(b) wherein the composition is characterized by controlled release; or
(c) wherein the composition is characterized by sustained release.

13. The method according to claim 1, further comprising causing the active agent-hydroxypropylβcyclodextrin inclusion complex to form a dendrimer.
